(19) United States
(12) Patent Application Publication
(10) Pub. No.: US 2025/0122506 A1
(43) Pub. Date: Apr. 17, 2025

(54) Modulation of TJPI Expression to Treat Liver Diseases

(71) Applicant: AGENCY FOR SCIENCE TECHNOLOGY AND RESEARCH, Singapore (SG)

(72) Inventors: Jianliang Xu, Singapore (SG); Walter Hunziker, Singapore (SG); Jaya Kausalya P, Singapore (SG)

(21) Appl. No.: 18/687,385

(22) PCT Filed: Sep. 16, 2022
(86) PCT No.: PCT/SG2022/050663

§ 371 Date: Feb. 28, 2024

(30) Foreign Application Priority Data
Sep. 16, 2021 (SG) 10202110245U
Dec. 1, 2021 (SG) 10202113389U

(51) Int. Cl. C12N 15/113; A61P 35/00

(52) U.S. Cl. CPC C12N 15/1138; A61P 35/00; C12N 2310/14

(57) ABSTRACT

Disclosed is a method for treating a liver disease in a subject comprising administering a Tjp1 inhibitor to the subject. Also disclosed are a kit and a nucleic acid encoding a Tjp1 inhibitor.

### CROSS-REFERENCE TO RELATED APPLICATIONS

This application claims the benefit of priority of Singapore patent application Ser. No. 10/202,110245U, filed 16 Sep. 2021, and Singapore patent application Ser. No. 10/202,113389U, filed 1 Dec. 2021, the contents of it being hereby incorporated by reference in its entirety for all purposes.

### FIELD OF THE INVENTION

The present invention generally relates to molecular biology. In particular, the present invention relates to a method of treating liver diseases by targeting Tjp1.

### BACKGROUND OF THE INVENTION

Liver diseases affect many patients worldwide every year. For example, liver cancer is the third most common cause of cancer death globally as well as in the Asia Pacific region. Liver diseases can cause different levels of damage to the liver. Some liver disease causes liver fibrosis where excessive connective tissue builds up in the liver. Others cause cirrhosis, which is a more severe form of liver fibrosis that results in the distortion of the liver architecture. These are often associated with a disruption of bile flow, which leads to liver diseases such as, but not limited to cholestasis. Currently, different therapies such as drug therapy, surgery or trans-arterial therapy are available for liver diseases. Despite advances in these diverse therapeutic options, successfully treating liver diseases remains an unmet clinical challenge. Therapeutic approaches allowing survival of the cells in the liver or replacement of liver would be of tremendous economic and social impact. In view of the above problems, there is a need to provide an alternative method for treating a liver disease in a subject.

### SUMMARY

In one aspect, there is provided a method for treating a liver disease in a subject, wherein the method comprises administering of a pharmaceutically effective amount of a Tjp1 inhibitor to the subject.

In another aspect, there is provided a method of regenerating a biliary system in a subject, wherein the method comprises administering of a pharmaceutically effective amount of a Tjp1 inhibitor to the subject.

In another aspect, there is provided a nucleic acid encoding a Tjp1 inhibitor, wherein the nucleic acid comprises at least 60%, or at least 80% identity to a sequence selected from a group consisting of 5′-CGTGGATTGAACTTACTAAAT-3′ (SEQ ID NO: 4), 5′-ATTTAGTAAGTTCAATCCACG-3′ (SEQ ID NO: 91), 5′-CCGCGAAGTTATGAGCAAGTT-3′ (SEQ ID NO: 5), 5′-AACTTGCTCATAACTTCGCGG-3′ (SEQ ID NO: 92), 5′-CGGCCATTTGAACGCAAATTT-3′ (SEQ ID NO: 6), 5′-AAATTTGCGTTCAAATGGCCG-3′ (SEQ ID NO: 93), 5′-GCAATGGTTAACGGAGTTTCA-3′ (SEQ ID NO: 104), 5′-AATGGTTAACGGAGTTTCAAT-3′ (SEQ ID NO: 105), 5′-AAGGAAATTTCACAAGATAGT-3′ (SEQ ID NO: 106), 5′-TACAAGTGATGACCTTGATTT-3′ (SEQ ID NO: 107), 5′-ACTGATCAAGAACTAGATGAA-3′ (SEQ ID NO: 108), 5′-AAGAACTAGATGAAACTCTTA-3′ (SEQ ID NO: 109), 5′-CCCACCTTTAGATAAAGAGAA-3′ (SEQ ID NO: 110), 5′-CAGCACGATTTCTGTTTAGAT-3′ (SEQ ID NO: 111), 5′-AGCACGATTTCTGTTTAGATA-3′ (SEQ ID NO: 112), and 5′-TAGATAATACACCACTACATT-3′ (SEQ ID NO: 113).

In another aspect, there is provided a nucleic acid encoding a Tjp1 inhibitor, wherein the nucleic acid comprises at least 60% identity to a sequence selected form the group consisting of:
i) a combination of SEQ ID NO: 4 and SEQ ID NO: 91, wherein SEQ ID NO: 4 is flanked at the 5′end by a nucleotide sequence comprising 1 to 10 nucleotides, wherein a nucleotide sequence of 1 to 20 nucleotides connects the 3′end of SEQ ID NO: 4 with the 5′end of SEQ ID NO: 91, and wherein SEQ ID NO: 91 is flanked at the 3′end by a nucleotide sequence comprising 1 to 10 nucleotides;
ii) a combination of SEQ ID NO: 5 and SEQ ID NO: 92, wherein SEQ ID NO: 5 is flanked at the 5′end by a nucleotide sequence comprising 1 to 10 nucleotides, wherein a nucleotide sequence of 1 to 20 nucleotides connects the 3′end of SEQ ID NO: 5 with the 5′end of SEQ ID NO: 92, and wherein SEQ ID NO: 92 is flanked at the 3′end by a nucleotide sequence comprising 1 to 10 nucleotides; and
iii) a combination of SEQ ID NO: 6 and SEQ ID NO: 93, wherein SEQ ID NO: 6 is flanked at the 5′end by a nucleotide sequence comprising 1 to 10 nucleotides, wherein a nucleotide sequence of 1 to 20 nucleotides connects the 3′end of SEQ ID NO: 6 with the 5′end of SEQ ID NO: 93, and wherein SEQ ID NO: 93 is flanked at the 3′end by a nucleotide sequence comprising 1 to 10 nucleotides.

In another aspect, there is provided a nucleic acid encoding a Tjp inhibitor, wherein the nucleic acid comprises at least 60% identity to a sequence selected from the group consisting of: 5′-CCGGCGTGGATTGAACTTACTAAATCTCGAGATTTAGTAAGTT CAATCCACGTTTTTG-3′ (SEQ ID NO: 1), 5′-CCGGCCGCGAAGTTATGAGCAAGTTCTCGAGAACTTGCTCATAACTTCGCGGTTTTTG-3′ (SEQ ID NO: 2), and 5′-CCGGCGGCCATTTGAACGCAAATTTCTCGAG AAATTTGCGTTCAAATGGCCGTTTTTG-3′ (SEQ ID NO: 3).

In another aspect, there is provided a nucleic acid encoding a Tjp inhibitor, wherein the nucleic acid comprises comprises at least 60% identity to a sequence selected from a group consisting of 5′-UGAAACUCCGUUAACCAUUGC-3′ (SEQ ID NO: 94), 5′-AUUGAAACUCCGUUAACCAUU-3′ (SEQ ID NO: 95), 5′-ACUAUCUUGUGAAAUUUCCUU-3′ (SEQ ID NO: 96), 5′-AAAUCAAGGUCAUCACUUGUA-3′ (SEQ ID NO: 97), 5′-UUCAUCUAGUUCUUGAUCAGU-3′ (SEQ ID NO: 98), 5′-UAAGAGUUUCAUCUAGUUCUU-3′ (SEQ ID NO: 99), 5′-UUCUCUUUAUCUAAAGGUGGG-3′ (SEQ ID NO: 100), 5′-AUCUAAACAGAAAUCGUGCUG-3′ ID 5′-(SEQ NO: 101), UAUCUAAACAGAAAUCGUGCU-3′ (SEQ ID NO: 102) and 5′-AAUGUAGUGGUGUAUUAUCUA-3′ (SEQ ID NO: 103).

In yet another aspect, there is provided a nucleic acid encoding a Tjp inhibitor, wherein the nucleic acid comprises a sequence selected from a group consisting of 5′-UGAAACUCCGUUAACCAUUGC-3′ (SEQ ID NO: 94), 5′-AUUGAAACUCCGUUAACCAUU-3′ (SEQ ID NO: 95), 5′-ACUAUCUUGUGAAAUUUCCUU-3′ (SEQ ID NO: 96), 5′-AAAUCAAGGUCAUCACUUGUA-3′ (SEQ ID NO: 97), 5′-UUCAUCUAGUUCUUGAUCAGU-3′ (SEQ ID NO: 98), 5′-UAAGAGUUUCAUCUAGUUCUU-3′ (SEQ ID NO: 99), 5′-UUCUCUUUAUCUAAAGGUGGG-3′ (SEQ ID NO: 100), 5′-AUCUAAACAGAAAUCGUGCUG-3′ (SEQ ID NO: 101), 5′-UAUCUAAACAGAAAUCGUGCU-3′ (SEQ ID NO: 102) and 5′-AAUGUAGUGGUGUAUUAUCUA-3′ (SEQ ID NO: 103).

In another aspect, there is provided a kit comprising the Tjp1 inhibitor as defined herein and/or the nucleic acid as defined herein.

### BRIEF DESCRIPTION OF THE DRAWINGS

The invention will be better understood with reference to the detailed description when considered in conjunction with the non-limiting examples and the accompanying drawings, in which:

FIG. 1 shows a series of experimental data comparing the effects in liver anatomy and function of Tjp1 conditional knockout (cKO) and control mice. FIG. 1A is a series of photos of immunofluorescence microscopy (IF) for Tjp1 (ZO-1) (left) and Tjp2 (ZO-2) (second from left), hematoxylin and eosin (H&E) and Sirius red staining. Tjp1 is absent from hepatocytes and cholangiocytes of the liver of Tjp1 cKO mice. Nuclei are labelled with DAPI (third from left) in the photos of immunofluorescence microscopy, and the merged images (right) represent superimposed images of the Tjp1 (ZO-1), Tjp2 (ZO-2) and DAPI images. H&E and Sirius red staining do not reveal apparent changes in liver histology or fibrosis, respectively. FIG. 1B shows 4 graphs representing the liver to body weight ratio, and levels of plasma bile acid (BA), alanine aminotransferase (ALT) and aspartate aminotransferase (AST) levels in control and Tjp1 cKO mice. Liver to body weight ratio, and plasma bile acid (BA), ALT and AST levels are comparable in control and Tjp1 cKO mice. FIG. 1C shows photos of immunofluorescence staining for Tjp1 and Tjp2. FIG. 1D shows an image of a Western blot analysis of Claudin-1 (Cldn1), Claudin-2 (Cldn2), Claudin-3 (Cldn3), Occludin, Cingulin (Cgn), E-cadherin and Vinculin in liver cells from control and Tjp1 cKO mice. The respective graph showing the relative protein expression is also provided. FIG. 1E shows photos of immunofluorescence microscopy for Cldn1, Cldn2, Cldn3 and Cingulin (Cgn) and carcinoembryonic antigen-related cell adhesion molecule (Ceacam). FIGS. 1C-1E show that conditional deletion of Tjp1 in mouse liver does not alter expression and localization of tight junction components. Western blot analysis. FIG. 1F shows electron microscopy (EM) images of the liver tissue from control and Tjp1 cKO mice. The images showing typical electron dense tight junction plaque (black box) in the vicinity of the bile canaliculi and microvilli Conditional deletion of Tjp1 has no apparent effect on tight junction morphology as assessed by EM. FIG. 1G is a graph showing the levels of 4 kDa FITC dextran in control, Tjp cKO and LPS-treated mice. 4 kDa FITC dextran was injected into the tail vein and its transfer to the bile assessed. Deletion of Tjp1 does not affect the bile-blood barrier. LPS-treated mice was used as positive control, wherein the bile-blood barrier was compromised by LPS injection, resulting in detectable FITC-dextran leakage. FIG. 1 shows that conditional deletion of Tjp1 in mouse liver has no apparent effect on liver histology or function.

FIG. 2 shows a series of experimental data showing the effects of thioacetamide (TAA) induced injury to the liver in the control and Tjp1 conditional knockout (cKO) mice. FIG. 2A shows images of the livers in TAA treated Tjp1 cKO and wild-type (WT) control mice. The gross anatomy of the liver in TAA treated Tjp1 cKO mice remains unchanged, whereas the liver of the WT mice exhibited some signs of damage as shown by the areas circled by white dotted lines. FIG. 2B is a line graph showing the survival of Tjp1 cKO and control mice after TAA injection. Tjp1 cKO mice subjected to prolonged TAA treatment do not show an increased mortality as observed in controls. FIG. 2C are graphs showing the levels of ALT and AST in Tjp1 cKO and control mice at 6, 24 and 48 hours after TAA treatment. Plasma ALT and AST levels decreased in TAA treated Tjp1 cKO mice as compared to controls, indicating that the hepatic deletion of Tjp1 attenuates liver injury. FIG. 2D is a compilation of images of H&E staining of control and Tjp1 cKO livers. The quantification of necrosis is provided in the accompanying graph. TAA-induced liver necrosis is significantly suppressed in Tjp1 cKO livers as compared to controls. The white arrow shows the necrotic area in the liver tissue. FIG. 2E shows images of H&E staining and graphs of plasma ALT and AST levels of Tjp1 cKO mice and control mice treated with TAA. Tjp1 deletion protects from chronic liver injury as assessed by biochemistry and H&E staining. Plasma ALT and AST levels, and liver histology were significantly less affected in Tjp1 cKO mice treated with TAA for 18 days as compared to corresponding controls. FIG. 2F shows images of Sirius red staining and a graph showing the quantification of the percentage of Sirius red positive area. Liver fibrosis after 18 days of TAA administration was reduced in Tjp1 cKO liver as compared to controls. FIG. 2G shows a series of column graphs that presents the expression levels of fibrosis markers monitored by qRT-PCR in the liver of chronically TAA-treated Tjp1 cKO and control wild type (WT) mice. The fibrosis markers are αSMA, CK19, collagen 1a1, osteopontin, TIMP1, TGFβ, CTGF, PDGFRβ and PDGFβ, and all the markers have lower expression levels in the liver of chronically TAA-treated Tjp1 cKO mice as compared to corresponding controls. FIG. 2H shows images of CK19 staining and a graph of the percentage CK19 positive area. Chronic TAA-induced CK19 staining is lower in Tjp1 cKO liver as compared to corresponding controls. FIG. 21 shows images of caspase 3 staining and a graph of the percentage caspase 3 positive area. A positive caspase 3 staining represents the presence of apoptosis. Chronic TAA exposure leads to fewer caspase-3 positive cells in the Tjp1 cKO liver as compared to corresponding controls, indicative to less cell death. Thus, FIG. 2 illustrates that the inactivation of Tjp1 protects mice from TAA induced acute and chronic liver injury.

FIG. 3 shows a series of experimental data illustrating the effects of 3,5-Diethoxycarbonyl-1,4-Dihydrocollidine (DDC) diet induced injury to the liver in the control and Tjp1 conditional knockout (cKO) mice. FIG. 3A shows images of the livers in DDC fed Tjp1 cKO and wild-type (WT) control mice. The Tjp1 cKO liver shows normal size and coloration as compared to the enlarged control liver from mice fed with DDC diet for 7 days. FIG. 3B shows line graphs quantifying the weight of the liver and spleen against body weight of Tjp1 cKO and control mice with and without DDC diet induced injury. The DDC diet induced increases in liver and spleen to body weight ratio in control mice. This increase in liver and spleen to body weight ratio is suppressed or abrogated in Tjp1 cKO animals. FIG. 3C is a compilation of images of H&E staining of control and Tjp1 cKO livers. After being fed DDC diet for 28 days, massive ductular reaction was observed in the liver of control mice, but not in Tjp1 cKO mice. FIG. 3D are graphs showing the levels of BA, plasma alkaline phosphatase (AP), ALT, AST and bilirubin in Tjp1 cKO and control mice with and without DDC diet. The levels of BA, AP, ALT, AST and bilirubin remains unchanged in Tjp1 cKO mice with DDC diet, showing that inactivation of Tjp1 protects the liver from DDC diet induced liver injury. FIG. 3E shows images of Sirius red staining and a graph showing the quantification of the percentage of Sirius red positive area. DDC diet induced liver fibrosis as assessed by Sirius red staining was reduced in Tjp1 cKO liver as compared to controls. FIG. 3F shows images of CK19 staining and a graph of the percentage CK19 positive area. DDC diet induced ductular reaction, monitored by staining for the cholangiocyte marker CK19, is suppressed in Tjp1 cKO liver. FIG. 3G shows a series of column graphs that presents the expression levels of fibrosis markers monitored by qRT-PCR in the liver of Tjp1 cKO and control wild type (WT) mice with and without DDC diet. The fibrosis markers are αSMA, CK19, epithelial cellular adhesion molecule (EpCam), collagen 1, TIMP1, TGFβ1, CTGF, PDGFRβ and PDGFβ, and all the markers have lower expression levels in the liver of DDC fed Tjp1 cKO mice as compared to the corresponding control. FIG. 3H shows an image of a Western blot analysis of αSMA, Smad2, phosphorylated SMAD2 (pSMAD2) in liver cells from control and Tjp1 cKO mice fed with standard chow, DDC diet for 7 and 28 days. GAPDH serves as a reference control. FIG. 3l shows photos of immunofluorescence staining for aSMA (left), DAPI (center) and merged (right) of liver samples obtained from DDC fed Tjp1 cKO and control mice. FIG. 31 shows immunohistochemistry images stained for laminin 1-2 and collagen 1 of liver samples obtained from DDC fed Tjp1 cKO and control mice. FIG. 3K shows a series of column graphs that presents the expression levels of macrophage markers CD11b and F4/80, and key inflammatory cytokines TNFα, interleukin 6 (IL-6) and osteopontin monitored by qRT-PCR in the liver of Tjp1 cKO and control wild type (WT) mice with and without DDC diet. The expression levels of the macrophage markers and inflammatory cytokines are lower in the liver of DDC fed Tjp1 cKO mice in comparison to the corresponding control. FIG. 3 illustrates that the inactivation of Tjp1 protects mice from DDC diet induced liver injury.

FIG. 4 shows a series of experimental data showing the effects of bile duct ligation (BDL) induced liver injury in control and Tjp1 conditional knockout (cKO) mice. FIG. 4A shows a column graph quantifying the mRNA expression levels of Tjp1 and Tjp2 in the liver of sham or BDL treated groups of control and Tjp1 cKO mice. FIG. 4B shows immunohistochemistry images of liver of sham or BDL treated groups of control and Tjp1 cKO mice. Graphs showing the percentage necrosis area, liver-to-body weight ratio, and plasma levels of ALT, AST are also presented. The BDL induced liver injury, as assessed by liver necrosis, liver-to-body weight ratio and plasma ALT and AST, are reduced in Tjp1 cKO animals. FIG. 4C shows images of Sirius red staining and a graph showing the quantification of the percentage of Sirius red positive area. BDL induced liver fibrosis as assessed by Sirius red staining was reduced in Tjp1 cKO liver as compared to controls. FIG. 4D shows a column graph of mRNA expression levels of inflammatory markers (collagen 1A, EPCAM, αSMA, MMP9, Timp-1, CTGF and TGFβ) in the liver of Tjp1 cKO and control wild type (WT) mice with and without BDL. The protein expression of TIMP is also shown in the Western blot, wherein the relative expression is presented in the column graph. The expression levels of the inflammatory markers are lower in the liver of BDL Tjp1 cKO mice in comparison to the corresponding control. FIG. 4E shows images of CK19 staining, a graph of the percentage CK19 positive area, and another graph quantifying the CK19 mRNA expression. BDL-induced ductular reaction, monitored by staining for the cholangiocyte marker CK19, is suppressed in Tjp1 cKO liver. FIG. 4F presents a series of staining images and quantification of F4/80 and CD11b positive macrophages and neutrophils to illustrate reduced liver immune cell infiltration in Tjp1 cKO mice after BDL. The other series of column graphs and images of Western blot to illustrate reduced expression levels of inflammatory cytokines (CCL21, C-reactive, endoglin, ICAM-1, IGFBP-1, MMP9, Myeloperoxidase, osteopontin, TNFα, IL-6, and IL-11b) in Tjp1 cKO mice after BDL. FIG. 4G shows graphs that present the expression levels of liver and plasma bile acids (BA), plasma AP and bilirubin, which shows reduced cholestasis and improved liver function in Tjp1 cKO mice after BDL. FIG. 4H shows graphs of total bile and bile acids in control and Tjp1 cKO mice after BDL. Lower bile acid concentration in the bile of Tjp1 cKO mice after BDL may contribute the reduced liver injury. FIG. 4I shows a series of column graphs that presents the expression levels of genes involved in bile acid synthesis (Cyp7a1, Cyp7b1, Cyp8b1, Cyp27a1, FXR and SHP-1) and bile acid transport (NTCP, Oatp1, Oatp2, ABCB11, ABCB4, ABCC2, ABCC3 and ABCC4). FIG. 4J shows a column graph of mRNA expression levels of Cyp3a11, Sult2a1, Ugt1a1, CAR and PXR in the liver of Tjp1 cKO and control wild type (WT) mice with and without BDL. FIG. 4K shows images of Ki67 and cleaved caspase 3 staining, as well as graphs of the number of Ki67 and cleaved caspase 3 positive cells. Increased hepatocyte proliferation (shown by Ki67 staining and quantification) and reduced apoptosis (shown by Caspase-3 staining and quantification) in the Tjp1 cKO mouse liver after BDL may contribute to reduced liver injury. FIG. 4 illustrates that the inactivation of Tjp1 protects mice from bile duct ligation (BDL) induced liver injury.

FIG. 5 shows a series of experimental data showing the effects of the inactivation of Tjp1 in the liver of Yap cKO mouse conditional knockout (cKO) mice. FIG. 5A shows immunohistochemistry images of liver of control and Tjp1 deletion in Yap cKO mice. Graphs showing the percentage necrosis area, liver and spleen to body weight ratio, and plasma bile acid and bilirubin are also presented. Liver specific Tjp1 deletion in the Yap cKO background improves the liver phenotype of the Yap cKO mouse. FIG. 5B shows images of Sirius red staining and a graph showing the quantification of the percentage of Sirius red positive area. Plasma ALT and AST expression levels are also assessed. Reduced plasma ALT and AST in Tjp1 inactivated livers shows improved healing to liver injury and reduced Sirus red levels show suppressed liver fibrosis. FIG. 5C shows column graphs of mRNA expression levels of fibrosis marker (αSMA, CK19, collagen 1, TIMP1, TGFβ, CTGF, EpCam, PDGFRβ and PDGFβ) in the liver of Yap cKO and control wild type (WT) mice with and without Tjp1 inactivation. FIG. 5D is an image of a Western blot showing protein expression levels of fibrosis marker (αSMA, Laminin 1-2 and osteopontin) in the liver of Yap cKO and control wild type (WT) mice with and without Tjp1 inactivation. GAPDH serves as a reference control. FIG. 5E shows images of immunohistochemistry staining of fibrosis marker (αSMA, collagen 1, and Laminin1-2) in the liver of Yap cKO and control wild type (WT) mice with and without Tjp1 inactivation. FIG. 5F is a series of column graphs to illustrate reduced expression levels of inflammatory cytokines (F4/80, CD11b, osteopontin, TNFα, IL-6, and IL-1b) in the liver of Yap cKO and control wild type (WT) mice with and without Tjp1 inactivation. FIG. 5G shows images of bile duct marker CK19 immunohistochemistry staining in the liver of Yap cKO and control wild type (WT) mice with and without Tjp1 inactivation. The biliary tree, which is absent from the Yap cKO liver, is restored in the Tjp1 Yap cKO liver. FIG. 5H shows graphs that present the liver and spleen to body weight ratio, and the levels of serum bile acids (BA), plasma AP ALT, AST and bilirubin. Prolonged Tjp1 inactivation further normalizes liver and spleen to body weight ratio, and further improves liver function as assessed by serum bile acid, levels. FIG. 5 illustrates that the inactivation of Tjp1 ameliorates the liver phenotype of the Yap cKO mouse.

FIG. 6A shows 3 exemplary short hairpin molecule sequences. The sequences targeting Tjp1 in the short hairpin molecule are in bold, and the remaining sequences are required for the short hairpin formation in the transcribed shRNA. These short hairpin molecules were selected based on the ability of the transcribed shRNAs to silence Tjp1 expression in tissue culture cells. The short hairpin molecules were incorporated into an AAV8 vector downstream of the hepatocyte specific thyroxine binding globulin (TBG) promotor to form different AAV8 TBG-shTjp1 vectors encoding for the Tjp1 shRNAs for selective expression in liver hepatocytes. FIG. 6B shows photos of immunofluorescence staining for Tjp1 in liver samples of mice injected with the different AAV8 TBG-shTjp1 vectors encoding for the Tjp1 shRNAs. A DNA encoding scrambled (AAV-Scr) was used as negative control. AAV vectors comprising DNA encoding Tjp1 shRNA #21 (AAV-Tjp1 #21), Tjp1 shRNA #22 (AAV-Tjp1 #22) and Tjp1 shRNA #13 (AAV-Tjp1 #13) showed reduced expression of Tjp1. FIG. 6C shows images of Sirius red staining of liver samples, and graphs of plasma AST and ALT levels. Tamoxifen inducible deletion of Tjp1 in Tjp1^{f/f} Alb-Cre^{ERT2} mice (e.g. Tjp1 icKO^{HC}) and injection of AAV-Tjp1 #21 can ameliorate liver fibrosis as shown by Sirius red staining. Plasma AST and ALT levels are also reduced in both groups, therefore showing that liver injury can be suppressed. Both the tamoxifen induced deletion and the AAV-Tjp1 #21 mediated silencing of Tjp1 specifically occur in hepatocytes. FIG. 6 illustrates that Tjp1 inactivation or silencing improves liver injury in the Mdr2 mouse model.

FIG. 7 shows images of livers and Sirius red staining of liver samples. Carcinogenesis was assessed based on the liver of mice at 12 months of age (P360). Tumors can be seen on the liver from ABCB4^{-/-} mouse, but none were seen on the liver from ABCB4^{-/-}Tjp1 cKO mouse. Liver fibrosis was assessed by Sirius red staining in liver tissues of mice at 6 months of age (P180), whereby reduced Sirius red staining was observed in the ABCB4^{-/-}Tjp1 cKO mouse liver, showing that Tjp1 inactivation suppressed liver fibrosis. FIG. 7 illustrates that inactivation of Tjp1 suppresses liver carcinogenesis in the Mdr2 (Abcb4) KO mouse model.

FIG. 8A shows photos of immunofluorescence staining of AAV8 (2×10⁷) expressing Tjp1 short hairpin molecules in the liver. Expression of Tjp1 in the liver of mice injected with AAV8 carrying either scrambled (Scr) control short hairpin molecule (AAV8 Scr) (top row) or Tjp1 short hairpin molecules (AAV8 Tjp1) (bottom row) is monitored using immunofluorescence microscopy. Tjp2 expression is not affected by the short hairpin molecule. The graph shows the relative Tjp1 mRNA expression obtained from and quantitative reverse transcription polymerase chain reaction (qRT-PCR) of the livers from the different groups. Data for DNA encoding shRNA #21 (SEQ ID NO: 2) was shown and similar results were obtained for the other short hairpin molecule. FIG. 8B shows graphs of blood biochemistry levels for control or Yap cKO mice injected with AAV8 Scr or AAV8 shTjp1 mice. The graphs showed a reduction of levels in plasma bile acids (BA) plasma, alkaline phosphatase (AP), alanine aminotransferase (ALT), aspartate aminotransferase (AST) and bilirubin in the AAV8 shTjp1 mice when compared to the AAV8 Scr mice, indicating improvements for the different parameters in AAV8 shTjp1 injected mice. FIG. 8C are photos of sirius red and Ck19 stained tissues to monitor fibrosis and the biliary system, respectively. AAV8 short hairpin molecule suppresses fibrosis and induces the reformation of the biliary system in Yap cKO mice. The graphs illustrate the levels of sirius red and Ck19 expressed. FIG. 8D are photos of sirius red staining and graphs showing the levels of AST and ALT plasma levels in Mdr2/Abcb4 KO mice injected with AAV8 Scr or AAV8 shTjp1 vectors. FIG. 8 illustrates the effect of AAV8 mediated delivery of Tjp1 short hairpin molecule expressed from a liver specific promoter, and shows that AAV8 expressing Tjp1 short hairpin molecules from a liver specific promoter are liver protective in the Yap cKO and the Mdr2/Abcb4 KO mouse models.

### DETAILED DESCRIPTION

The liver is a metabolic hub responsible for physiologic functions including amino acid, carbohydrate, and lipid metabolism; detoxification; and bile secretion. Enterohepatic circulation of metabolites is required for digestion and metabolic homeostasis. Bile acid (BA) synthesis occurs in hepatocytes, and drainage from the liver is performed by cholangiocyte-lined bile ducts. Hepatocytes and cholangiocytes rely on tight junctions (TJs) to establish the blood-bile barrier (BBB) that segregates bile from the blood circulation. TJs are also thought to reinforce cell polarity by maintaining the segregation of distinct proteins to the apical and basolateral membranes. The polarized distribution of specific transporters contributes to the BBB because it is critical for the directional collection of BAs from the blood and their release into the bile by hepatocytes, or the concentration of bile in the bile ducts.

The dysregulation of any one of the liver functions described above can lead to many different liver diseases. Liver diseases are major causes of illness and mortality worldwide. Liver diseases can ultimately lead to liver failure, which is a life-threatening condition. Despite the availability of different drugs and treatments to treat liver disease, the outcomes remain dismal. For example, ursodeoxycholic acid (UDCA) has been the accepted therapy for a liver disease known as primary biliary cholangitis (PBC) since 2016. However, the efficacy remains debatable as it does not provide any improvements to survival or liver histology. Presently, effective medical therapy for patients suffering from liver diseases and injury remains to be unmet. In view of the above, there is a need to provide an alternative method for treating a liver disease in a subject and to regenerate a biliary system in a subject.

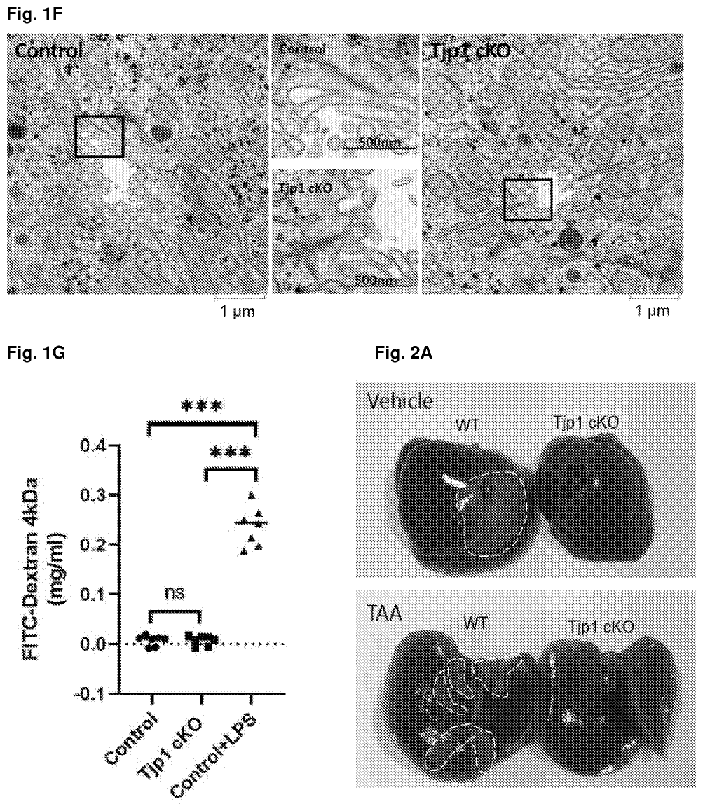

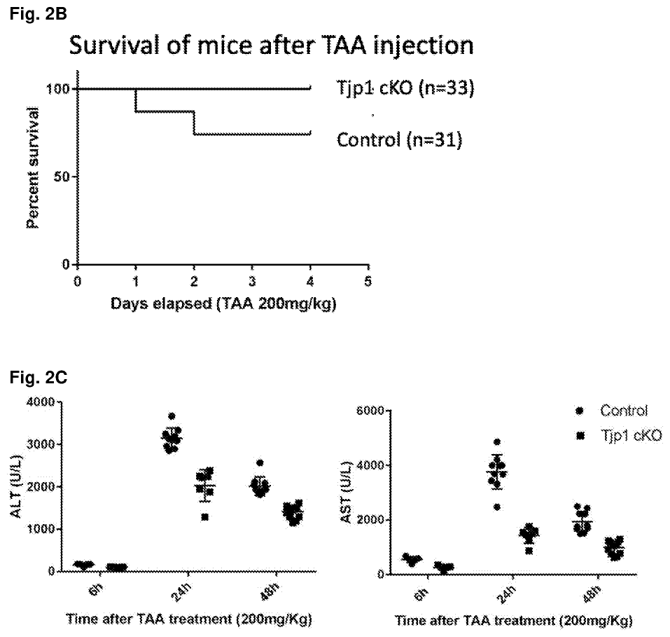

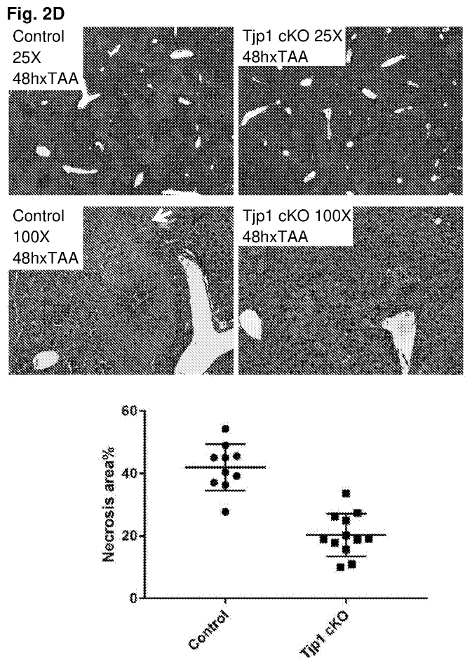

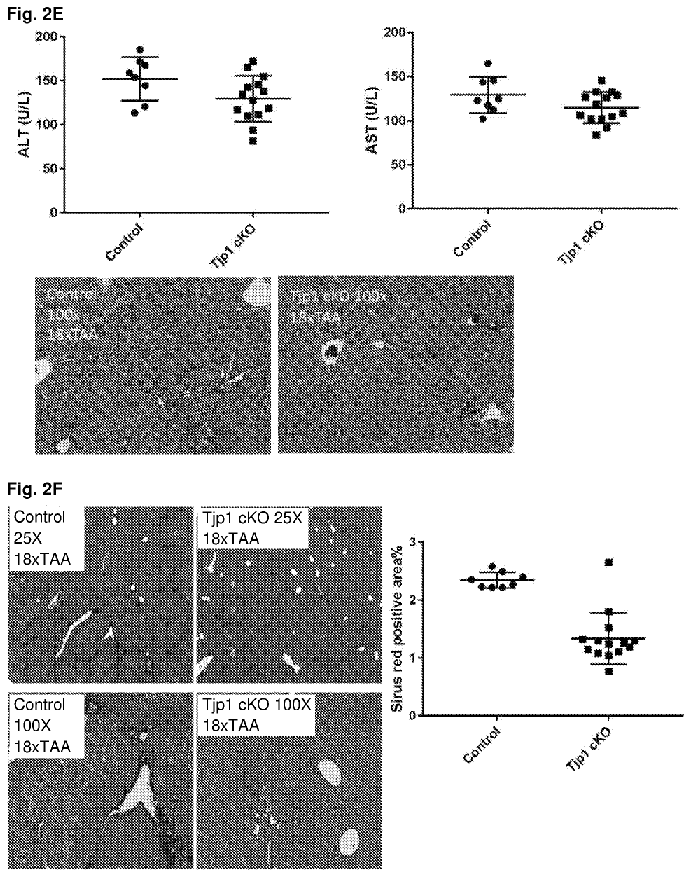

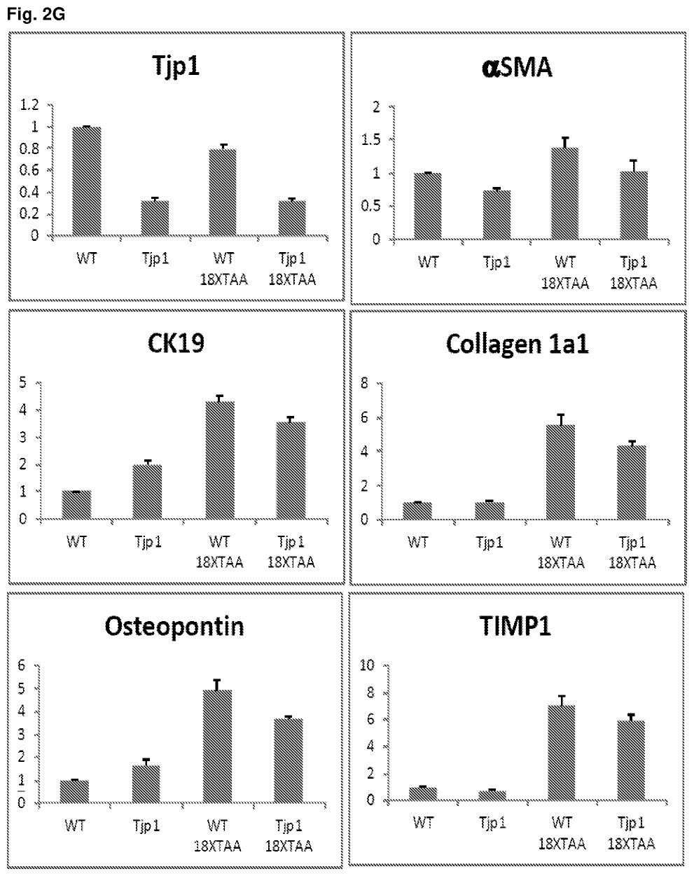

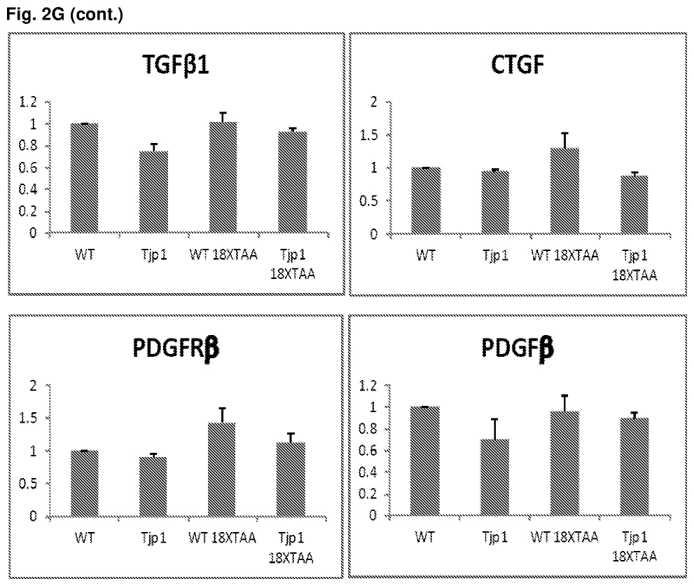

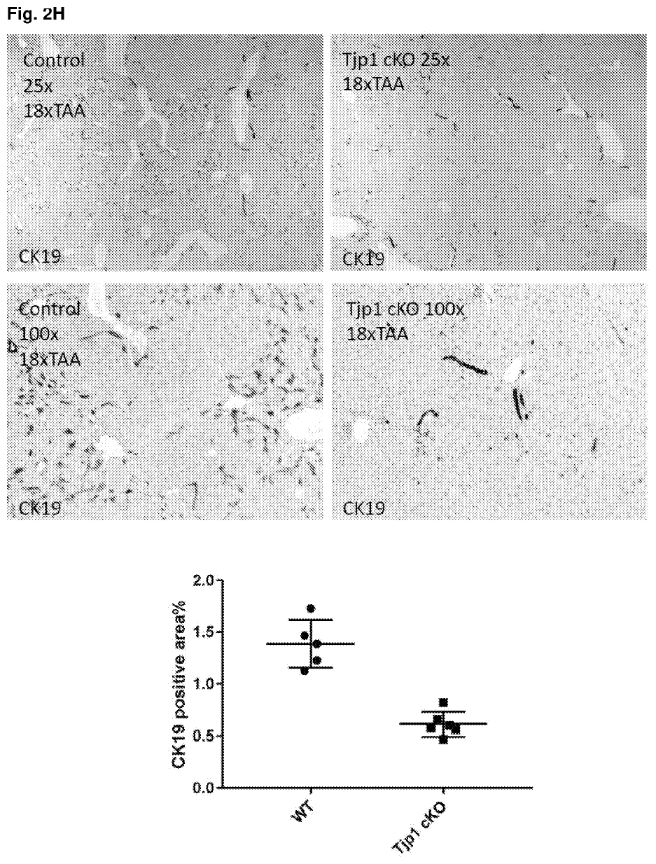

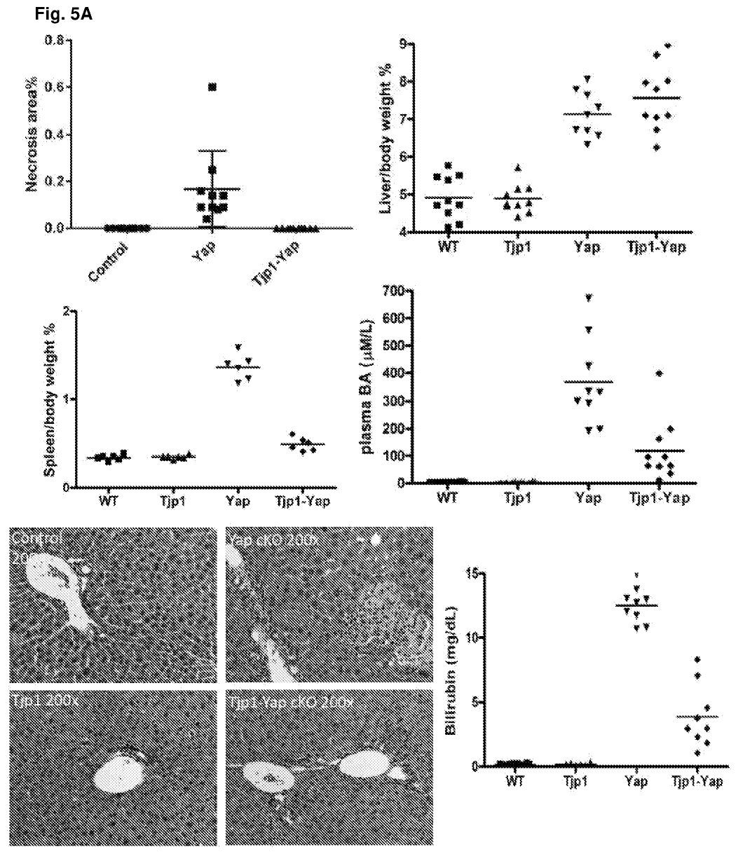

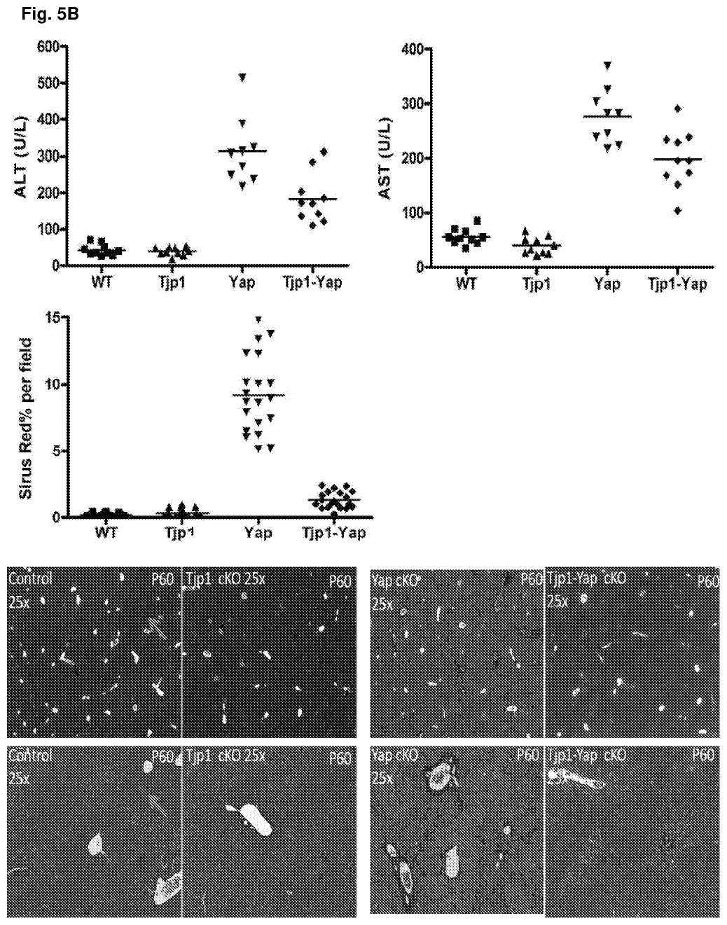

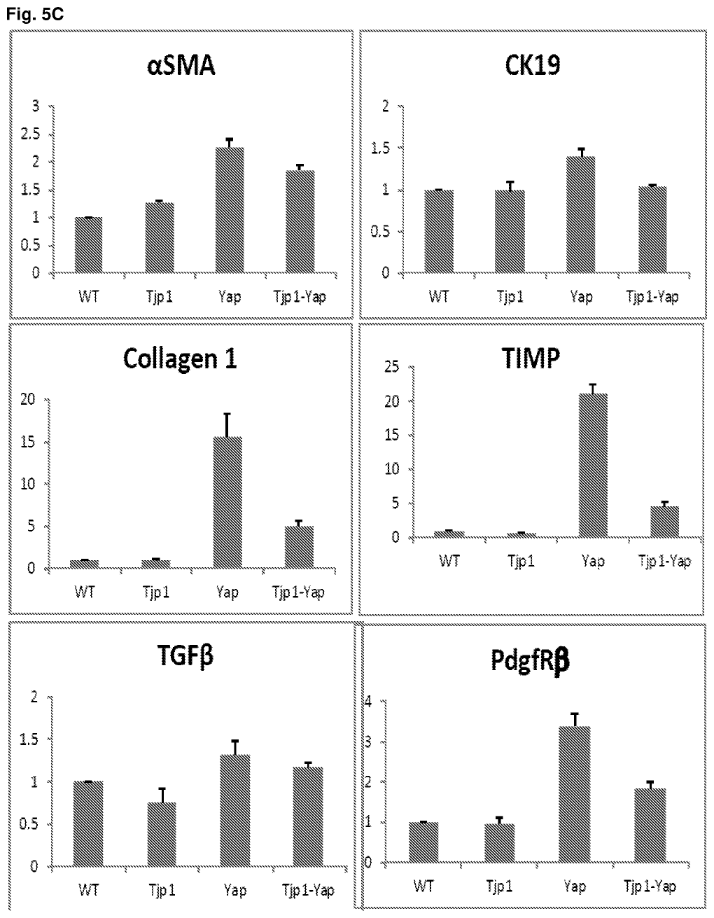

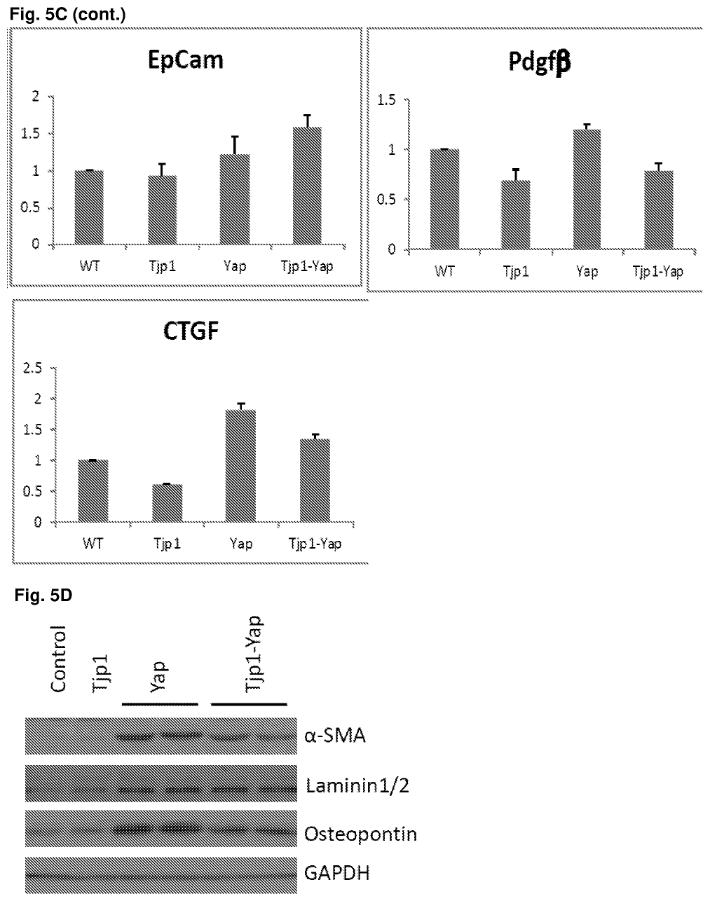

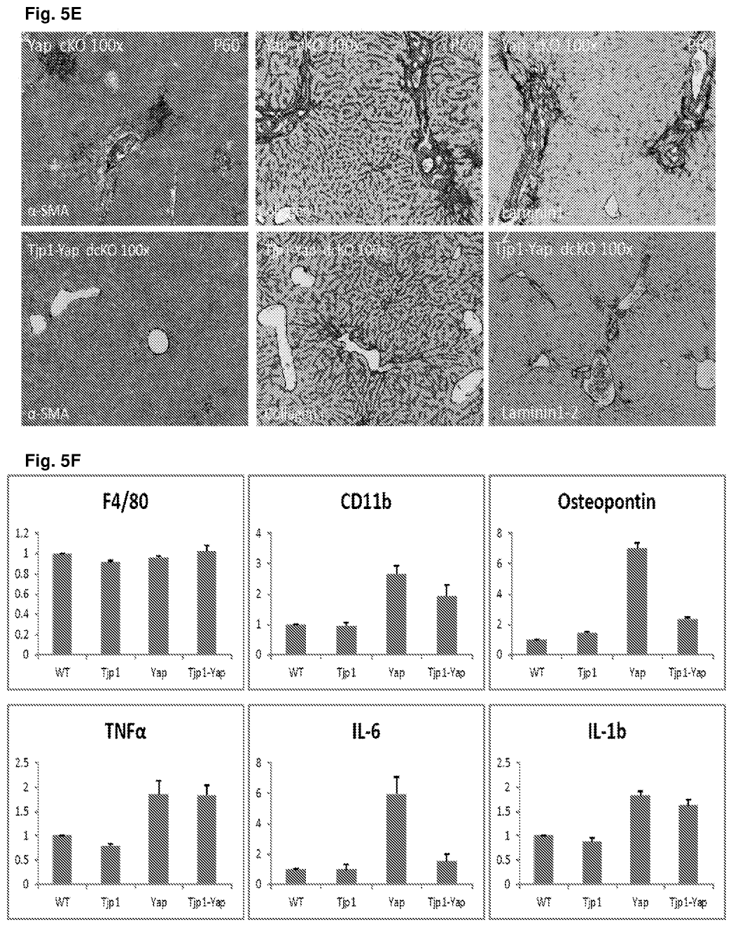

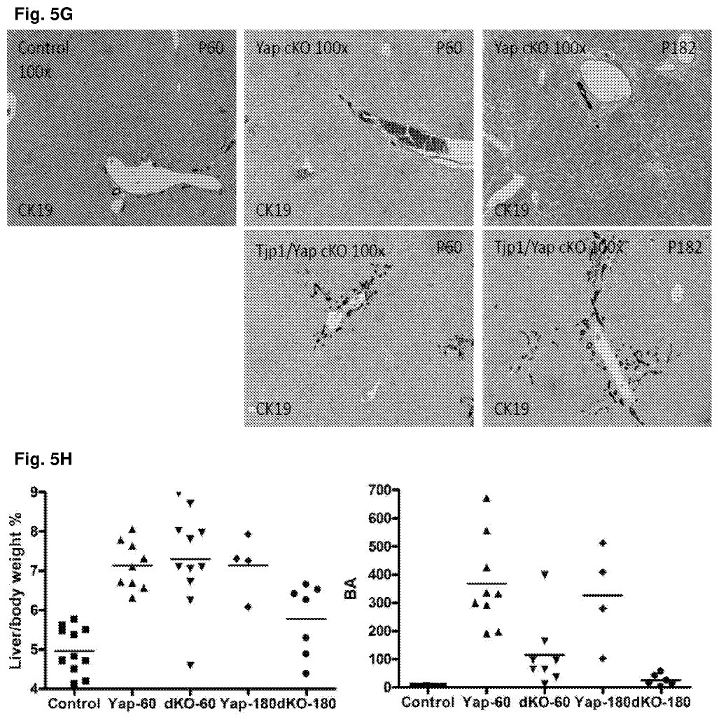

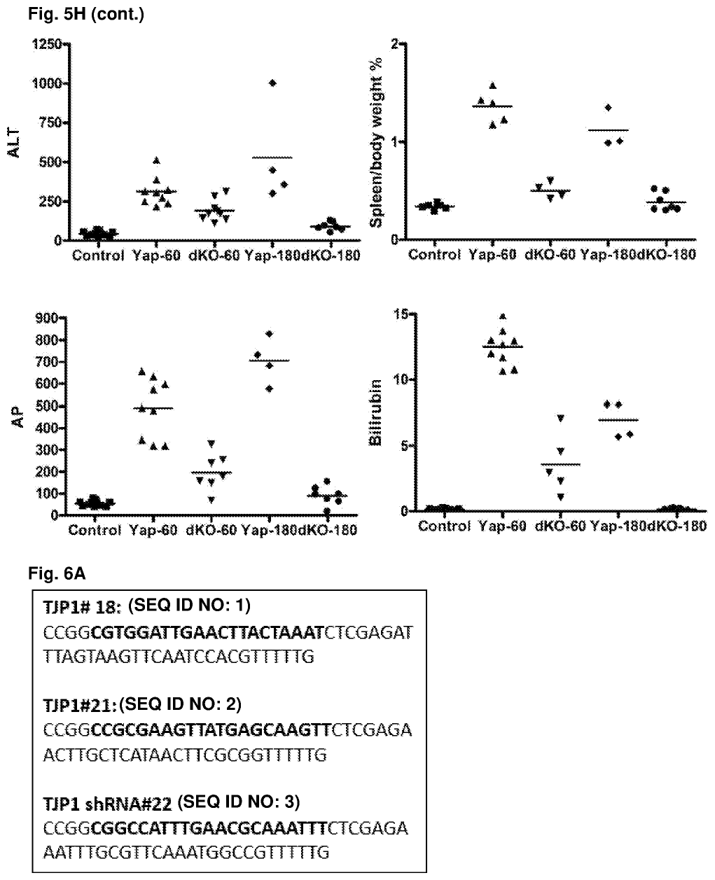

The inventors of the present disclosure have found an alternative method for treating a liver disease in a subject. Thus, in one aspect, the present invention provides a method for treating a liver disease in a subject comprises administering of (a pharmaceutically effective amount of) a Tjp1 inhibitor to the subject. The administration of a Tjp1 inhibitor inhibits Tjp1 expression. In some examples, the subject has had a liver disease or suffered from a liver injury. Without being bound by theory, the Tjp1 inhibition reduces recruitment of immune cells such as macrophage and neutrophil, and also reduces levels of inflammatory cytokines and chemokines (FIGS. 2-4). This decreases the inflammatory reaction that takes place after a liver disease or injury and allows the process of regeneration of biliary system to take place in the liver (FIG. 5).

As used herein the terms Tjp1 (Tight Junction Protein 1) and ZO-1 (Zonula Occludens-1) are used interchangeably. The terms “Tjp1” and “ZO-1” refers to an actin-binding scaffold protein that is associated with tight junctions, which are critical for the biliary-blood barrier and in human patients. Exemplary nucleic acid sequences of Tjp1 include, but are not limited to SEQ ID NO: 7, 8, 10, 11, 114 or 115. Exemplary amino acid sequences of Tjp1 include, but are not limited to SEQ ID NOs: 9 or 12.

In one example, the Tjp1 inhibitor described herein inhibits Tjp1 expression. The term “inhibit” is used herein generally to mean a decrease of the amount as compared to an untreated subject or a control. However, for avoidance of doubt, “inhibit” means a decrease of Tjp1 levels sufficient to cause any improvement in a subject suffering from liver disease. In one example, inhibition means a decrease by at least 10% as compared to an untreated subject, or at least about 20%, or at least about 30%, or at least about 40%, or at least about 50%, or at least about 60%, or at least about 70%, or at least about 80%, or at least about 90% or up to and including a 100% decrease of Tjp1 expression as compared to an untreated subject, or any decrease between 10-100% of Tjp1 expression as compared to an untreated subject. As used herein, the term “control” refers to a population that is statistically similar to the set being tested, on which no changes are implemented. A non-limiting example of control as used herein is wild type mice (i.e. mice without Tjp1 knock-out).

It would be appreciated that any methods known in the art to be capable of reducing or inactivating Tjp1 expression or activity would be suitable for use in the methods as described herein. A person skilled in the art will appreciate that the list of the types of Tjp1 inhibitors included herein is not exhaustive. The examples of the types of Tjp1 inhibitors include, but are not limited to, a small molecule, an antibody, a polypeptide, a nucleic acid such as, but not limited to DNA, complementary DNA, siRNA or shRNA, and any other biological or chemical entity capable of inhibiting Tjp1 expression, function or activity. In one example, Tjp1 inhibitor includes, but is not limited to, a nucleic acid, a small molecule, an antibody, a polypeptide, and the like. A person skilled in the art will also appreciate that in some examples, the antibody that can be used as Tjp1 inhibitor is a monoclonal antibody, a polyclonal antibody, a recombinant antibody, a human antibody, a humanized antibody, a chimeric antibody, a multispecific antibody, or an antibody fragment thereof. In some examples, the antibody include, but is not limited to, a Fab fragment, a Fab′ fragment, a F(ab′) 2 fragment, a Fv fragment, a diabody, or a single chain antibody molecule. In some examples, the antibody is a human antibody.

As would be appreciated by the person skilled in the art, nucleic acid sequences such as short hairpin molecules, vectors, DNA inserts, shRNAs can be designed to target Tjp1. In one example, the nucleic acid that is used as Tjp1 inhibitor includes, but is not limited to, a short hairpin molecule, an shRNA, an siRNA, an antisense oligonucleotide (AON), a gapmer, a short hairpin Antisense Oligonucleotide (shAON), and the like. The term “short hairpin molecule” as used herein refers to an artificial nucleic acid sequence that has a hairpin-like structure with 5′- and 3′ tails. The short hairpin molecule can be a DNA sequence, an RNA sequence, or combinations thereof. The short hairpin molecule usually comprises a sequence that is identical or complementary to a sequence or part of a sequence of a target gene. In one example, the short hairpin molecule can be a DNA sequence that is inserted into a vector, such as plasmid vector or a viral vector, from which a short hairpin RNA is produced in the cell. A person skilled in the art would appreciate that the short hairpin molecule will be transcribed and processed by the cellular machinery in the cell into a molecule that can bind to a region in the mRNA of the target (for example, an mRNA that may be translated as Tjp1 protein). This will lead to the degradation of the target mRNA and prevent production of the Tjp1 protein, thereby silencing target gene expression via RNA interference. In another example, the nucleic acid is further transcribed into a short hairpin RNA (shRNA) and processed so that it binds to an mRNA encoding Tjp1 or a homolog thereof. In another example, when a nucleic acid is used as a Tjp1 inhibitor, the nucleic acid may be transcribed into a shRNA that is processed and binds to or interacts with mRNA which encodes Tjp1 and forms a shRNA-mRNA complex with the mRNA encoding Tjp1. In one example, when the nucleic acid that is used as Tjp1 inhibitor is transcribed into a shRNA and processed to bind to or interact with the mRNA that encodes the Tjp1 and forms a nucleic acid-mRNA complex, the mRNA in the shRNA-mRNA complex is cleaved and/or is not translated.

The term “homolog” as used herein refers to the existence of shared ancestry between genes (or structures) in different taxa. In other words, homology is the relationship between biological structures or sequences that are derived from a common ancestor and that ultimately have the same or similar functions (that is a biological equivalent). In terms of sequence homology, DNA or protein sequences are defined in terms of shared ancestry. The term “sequence homology” is often used in place of the term “sequence similarity”, or vice versa. In one example, such sequence similarity can include the ability of the sequence to bind and modulate the function of the target, for example but not limited to binding and modulating the function of a Tjp1 mRNA using a short hairpin molecule, shRNA, siRNA, antisense oligonucleotide (AON), gapmer, or short hairpin antisense oligonucleotide (shAON). Thus, “homolog” refers to a sequence that is highly conserved (a conservative sequence) if it does not change or only shows minimal changes between the species.

The Tjp1 inhibitor can be a nucleic acid sequence that is identical or complementary to a Tjp1 coding sequence, either in its entirety or in part. The Tjp1 inhibitor can also be a nucleic acid sequence that is capable of inhibiting Tjp1. In one example, the Tjp1 inhibitor is a nucleic acid that has at least 60%, at least 65%, at least 70%, at least 75%, at least 80%, at least 85%, at least 90%, or at least 95% identity to a sequence that is capable of inhibiting Tjp1. In one example, the Tjp inhibitor is a nucleic acid sequence comprising part of a Tjp1 sequence that is, but not limited to 5′-CGTGGATTGAACTTACTAAAT-3′ (SEQ ID NO: 4), 5′-ATTTAGTAAGTTCAATCCACG-3′ (SEQ ID NO: 91), 5′-CCGCGAAGTTATGAGCAAGTT-3′ (SEQ ID NO: 5), 5′AACTTGCTCATAACTTCGCGG-3′ (SEQ ID NO: 92), 5′-CGGCCATTTGAACGCAAATTT-3′ (SEQ ID NO: 6), 5′-AAATTTGCGTTCAAATGGCCG-3′ (SEQ ID NO: 93), 5′-GCAATGGTTAACGGAGTTTCA-3′ (SEQ ID NO: 104), 5′-AATGGTTAACGGAGTTTCAAT-3′ (SEQ ID NO: 105), 5′-AAGGAAATTTCACAAGATAGT-3′ (SEQ ID NO: 106), 5′-TACAAGTGATGACCTTGATTT-3′ (SEQ ID NO: 107), 5′-ACTGATCAAGAACTAGATGAA-3′ (SEQ ID NO: 108), 5′-AAGAACTAGATGAAACTCTTA-3′ (SEQ ID NO: 109), 5′-CCCACCTTTAGATAAAGAGAA-3′ (SEQ ID NO: 110), 5′-CAGCACGATTTCTGTTTAGAT-3′ (SEQ ID NO: 111), 5′-AGCACGATTTCTGTTTAGATA-3′ (SEQ ID NO: 112), or 5′-TAGATAATACACCACTACATT-3′ (SEQ ID NO: 113). In another example, the Tjp1 inhibitor is a nucleic acid sequence comprising at least 60%, at least 65%, at least 70%, at least 75%, at least 80%, at least 85%, at least 90%, or at least 95% identity to a sequence that is, but not limited to 5′-CGTGGATTGAACTTACTAAAT-3′ (SEQ ID NO: 4), 5′-ATTTAGTAAGTTCAATCCACG-3′ (SEQ ID NO: 91), 5′-CCGCGAAGTTATGAGCAAGTT-3′ (SEQ ID NO: 5), 5′AACTTGCTCATAACTTCGCGG-3′ (SEQ ID NO: 92), 5′-CGGCCATTTGAACGCAAATTT-3′ (SEQ ID NO: 6), 5′-AAATTTGCGTTCAAATGGCCG-3′ (SEQ ID NO: 93), 5′-GCAATGGTTAACGGAGTTTCA-3′ (SEQ ID NO: 104), 5′-AATGGTTAACGGAGTTTCAAT-3′ (SEQ ID NO: 105), 5′-AAGGAAATTTCACAAGATAGT-3′ (SEQ ID NO: 106), 5′-TACAAGTGATGACCTTGATTT-3′ (SEQ ID NO: 107), 5′-ACTGATCAAGAACTAGATGAA-3′ (SEQ ID NO: 108), 5′-AAGAACTAGATGAAACTCTTA-3′ (SEQ ID NO: 109), 5′-CCCACCTTTAGATAAAGAGAA-3′ (SEQ ID NO: 110), 5′-CAGCACGATTTCTGTTTAGAT-3′ (SEQ ID NO: 111), 5′-AGCACGATTTCTGTTTAGATA-3′ (SEQ ID NO: 112), or 5′-TAGATAATACACCACTACATT-3′ (SEQ ID NO: 113).

In another example, the Tjp1 inhibitor is a nucleic acid not limited to i) a combination of SEQ ID NO: 4 and SEQ ID NO: 91, wherein SEQ ID NO: 4 is flanked at the 5′end by a nucleotide sequence comprising 1 to 10 nucleotides, wherein a nucleotide sequence of 1 to 20 nucleotides connects the 3′end of SEQ ID NO: 4 with the 5′end of SEQ ID NO: 91, and wherein SEQ ID NO: 91 is flanked at the 3′end by a nucleotide sequence comprising 1 to 10 nucleotides; ii) a combination of SEQ ID NO: 5 and SEQ ID NO: 92, wherein SEQ ID NO: 5 is flanked at the 5′end by a nucleotide sequence comprising 1 to 10 nucleotides, wherein a nucleotide sequence of 1 to 20 nucleotides connects the 3′end of SEQ ID NO: 5 with the 5′end of SEQ ID NO: 92, and wherein SEQ ID NO: 92 is flanked at the 3′end by a nucleotide sequence comprising 1 to 10 nucleotides; or iii) a combination of SEQ ID NO: 6 and SEQ ID NO: 93, wherein SEQ ID NO: 6 is flanked at the 5′end by a nucleotide sequence comprising 1 to 10 nucleotides, wherein a nucleotide sequence of 1 to 20 nucleotides connects the 3′end of SEQ ID NO: 6 with the 5′end of SEQ ID NO: 93, and wherein SEQ ID NO: 93 is flanked at the 3′end by a nucleotide sequence comprising 1 to 10 nucleotides. In another example, the Tjp1 inhibitor is a nucleic acid comprising at least 60%, at least 65%, at least 70%, at least 75%, at least 80%, at least 85%, at least 90%, or at least 95% identity to i) a combination of SEQ ID NO: 4 and SEQ ID NO: 91, wherein SEQ ID NO: 4 is flanked at the 5′end by a nucleotide sequence comprising 1 to 10 nucleotides, wherein a nucleotide sequence of 1 to 20 nucleotides connects the 3′end of SEQ ID NO: 4 with the 5′end of SEQ ID NO: 91, and wherein SEQ ID NO: 91 is flanked at the 3′end by a nucleotide sequence comprising 1 to 10 nucleotides; ii) a combination of SEQ ID NO: 5 and SEQ ID NO: 92, wherein SEQ ID NO: 5 is flanked at the 5′end by a nucleotide sequence comprising 1 to 10 nucleotides, wherein a nucleotide sequence of 1 to 20 nucleotides connects the 3′end of SEQ ID NO: 5 with the 5′end of SEQ ID NO: 92, and wherein SEQ ID NO: 92 is flanked at the 3′end by a nucleotide sequence comprising 1 to 10 nucleotides; or iii) a combination of SEQ ID NO: 6 and SEQ ID NO: 93, wherein SEQ ID NO: 6 is flanked at the 5′end by a nucleotide sequence comprising 1 to 10 nucleotides, wherein a nucleotide sequence of 1 to 20 nucleotides connects the 3′end of SEQ ID NO: 6 with the 5′end of SEQ ID NO: 93, and wherein SEQ ID NO: 93 is flanked at the 3′end by a nucleotide sequence comprising 1 to 10 nucleotides. In another example, SEQ ID NOs 4, 5 or 6 is flanked at the 5′end by a nucleotide sequence comprising 3 to 8 nucleotides, 4 to 6 nucleotides, or 1, 2, 3, 4, 5, 6, 7, 8, 9, or 10 nucleotides; a nucleotide sequence of 4 to 15 nucleotides, 8 to 13 nucleotides, or 1, 2, 3, 4, 5, 6, 7, 8, 9, 10, 11, 12, 13, 14, 15, 16, 17, 18, 19 or 20 nucleotides connect the 3′end of SEQ ID NO: 4, 5 or 6 with the 5′end of SEQ ID NO: 91, 92 or 93 respectively; and SEQ ID NOs 91, 92 or 93 is flanked at the 3′end by a nucleotide sequence comprising 3 to 8 nucleotides, 4 to 6 nucleotides, or 1, 2, 3, 4, 5, 6, 7, 8, 9, or 10 nucleotides. In yet another example, the Tjp1 inhibitor is a nucleic acid not limited to i) a combination of SEQ ID NO: 4 and SEQ ID NO: 91, wherein SEQ ID NO: 4 is flanked at the 5′end by a nucleotide sequence comprising 4 nucleotides, wherein a nucleotide sequence of 6 nucleotides connects the 3′end of SEQ ID NO: 4 with the 5′end of SEQ ID NO: 91, and wherein SEQ ID NO: 91 is flanked at the 3′end by a nucleotide sequence comprising 6 nucleotides; ii) a combination of SEQ ID NO: 5 and SEQ ID NO: 92, wherein SEQ ID NO: 5 is flanked at the 5′end by a nucleotide sequence comprising 4 nucleotides, wherein a nucleotide sequence of 6 nucleotides connects the 3′end of SEQ ID NO: 5 with the 5′end of SEQ ID NO: 92, and wherein SEQ ID NO: 92 is flanked at the 3′end by a nucleotide sequence comprising 6 nucleotides; or iii) a combination of SEQ ID NO: 6 and SEQ ID NO: 93, wherein SEQ ID NO: 6 is flanked at the 5′end by a nucleotide sequence comprising 4 nucleotides, wherein a nucleotide sequence of 6 nucleotides connects the 3′end of SEQ ID NO: 6 with the 5′end of SEQ ID NO: 93, and wherein SEQ ID NO: 93 is flanked at the 3′end by a nucleotide sequence comprising 6 nucleotides.

In yet another example, the Tjp1 inhibitor is a nucleic acid, but not limited to 5′-CCGGCGTGGATTGAACTTACTAAATCTCGAGATTTAGTAAGTTCAATCCAC GTTTTTG-′3 (SEQ ID NO: 1), 5′-CCGGCCGCGAAGTTATGAGCAAGTTCTCGAGAACTTGCTCATAACTTCGCGGTTTTTG-3′ (SEQ ID NO: 2), or 5′-CCGGCGGCCATTTGAACGCAAATTTCTCGAGAAATTTGCGTTCAAATGGCCGTTTTTG-3′ (SEQ ID NO: 3). It can be seen that the residues in bold in SEQ ID NOs: 1-3 are identical to the sequences in SEQ ID NOs: 4-6 and 91-93. SEQ ID NOs: 4-6 correspond to the Tjp1 coding sequence, and SEQ ID NOs: 91-93 correspond to the complementary sequences, wherein the complementary sequences are responsible for binding to the Tjp1 mRNA, thereby inhibiting Tjp1. The remaining sequences in SEQ ID NOs: 1-3 are required to create the short hairpin formation in the short hairpin molecule and can be any sequence that is capable of forming the short hairpin molecule. In yet another example, the Tjp1 inhibitor is a nucleic acid comprising at least 60%, at least 65%, at least 70%, at least 75%, at least 80%, at least 85%, at least 90%, at least 95%, at least 96%, at least 97%, at least 98%, at least 99%, or 100% identity to that is, but not limited to 5′-a sequence CCGGCGTGGATTGAACTTACTAAATCTCGAGATTTAGTAAGTTCAATCCACGTTTTTG-3′ (SEQ ID NO: 1), 5′-CCGGCCGCGAAGTTATGAGCAAGTTCTCGAGAACTTGCTCATAACTTCGCGGTTTTTG-3′ (SEQ ID NO: 2), or 5 CCGGCGGCCATTTGAACGCAAATTTCTCGAGAAATTTGCGTTCAAATGGCCGTTTTTG-3′ (SEQ ID NO: 3). In yet another example, the Tjp1 inhibitor is a short hairpin molecule comprising the sequence that is, but not limited to 5′-CCGGCGTGGATTGAACTTACTAAATCTCGAGATTTAGTAAGTTCAATCCACGTTTTTG-3′ (SEQ ID NO: 1), 5′-CCGGCCGCGAAGTTATGAGCAAGTTCTCGAGAACTTGCTCATAACTTCGCGGTTTTTG-3′ (SEQ ID NO: 2), or 5′-CCGGCGGCCATTTGAACGCAAATTTCTCGAGAAATTTGCGTTCAAATGGCCGTTTTTG-3′ (SEQ ID NO: 3). In yet another example, the Tjp1 inhibitor is a short hairpin molecule of at least 60%, at least 65%, at least 70%, at least 75%, at least 80%, at least 85%, at least 90%, at least 96%, at least 97%, at least 98%, at least 99%, or 100% identity to a sequence that is, but not limited to 5′-CCGGCGTGGATTGAACTTACTAAATCTCGAGATTTAGTAAGTTCAATCCACGTTTTTG-3′ (SEQ ID NO: 1), 5′-CCGGCCGCGAAGTTATGAGCAAGTTCTCGAGAACTTGCTCATAACTTCGCGGTTTTTG-3′ (SEQ ID NO: 2), or 5′-CCGGCGGCCATTTGAACGCAAATTTCTCGAGAAATTTGCGTTCAAATGGCCGTTTTTG-3′ (SEQ ID NO: 3).

In yet another example, the Tjp1 inhibitor is an siRNA comprising a sequence of, but not limited to 5′-UGAAACUCCGUUAACCAUUGC-3′ (SEQ ID NO: 94), 5′-AUUGAAACUCCGUUAACCAUU-3′ (SEQ ID NO: 95), 5′-ACUAUCUUGUGAAAUUUCCUU-3′ (SEQ ID NO: 96), 5′-AAAUCAAGGUCAUCACUUGUA-3′ (SEQ ID NO: 97), 5′-UUCAUCUAGUUCUUGAUCAGU-3′ (SEQ ID NO: 98), 5′-UAAGAGUUUCAUCUAGUUCUU-3′ (SEQ ID NO: 99), 5′-UUCUCUUUAUCUAAAGGUGGG-3′ (SEQ ID NO: 100), 5′-AUCUAAACAGAAAUCGUGCUG-3′ (SEQ ID NO: 101), 5′-UAUCUAAACAGAAAUCGUGCU-3′ (SEQ ID NO: 102) or 5′-AAUGUAGUGGUGUAUUAUCUA-3′ (SEQ ID NO: 103). In yet another example, the Tjp1 inhibitor is a nucleic acid comprising at least 60%, at least 65%, at least 70%, at least 75%, at least 80%, at least 85%, at least 90%, at least 95%, at least 96%, at least 97%, at least 98%, at least 99%, or 100% identity to a sequence that is, but not limited to 5′-UGAAACUCCGUUAACCAUUGC-3′ (SEQ ID NO: 94), 5′-AUUGAAACUCCGUUAACCAUU-3′ (SEQ ID NO: 95), 5′-ACUAUCUUGUGAAAUUUCCUU-3′ (SEQ ID NO: 96), 5′-AAAUCAAGGUCAUCACUUGUA-3′ (SEQ ID NO: 97), 5′-UUCAUCUAGUUCUUGAUCAGU-3′ (SEQ ID NO: 98), 5′-UAAGAGUUUCAUCUAGUUCUU-3′ (SEQ ID NO: 99), 5′-UUCUCUUUAUCUAAAGGUGGG-3′ (SEQ ID NO: 100), 5′-AUCUAAACAGAAAUCGUGCUG-3′ (SEQ ID NO: 101), 5′-UAUCUAAACAGAAAUCGUGCU-3′ (SEQ ID NO: 102) or 5′-AAUGUAGUGGUGUAUUAUCUA-3′ (SEQ ID NO: 103).

The present invention also provides a nucleic acid encoding a Tjp1 inhibitor, wherein the nucleic acid has at least 60%, at least 65%, at least 70%, at least 75%, at least 80%, at least 85%, at least 90%, or at least 95% identity to a sequence that is capable of inhibiting Tjp1. In another example, the nucleic acid comprises part of a Tjp1 sequence that is, but not limited to 5′-CGTGGATTGAACTTACTAAAT-3′ (SEQ ID NO: 4), 5′-ATTTAGTAAGTTCAATCCACG-3′ (SEQ ID NO: 91), 5′ CCGCGAAGTTATGAGCAAGTT-3′ (SEQ ID NO: 5), 5′-AACTTGCTCATAACTTCGCGG-3′ (SEQ ID NO: 92), 5′-CGGCCATTTGAACGCAAATTT-3′ (SEQ ID NO: 6), 5′-AAATTTGCGTTCAAATGGCCG-3′ (SEQ ID NO: 93), 5′-GCAATGGTTAACGGAGTTTCA-3′ (SEQ ID NO: 104), 5′-AATGGTTAACGGAGTTTCAAT-3′ (SEQ ID NO: 105), 5′-AAGGAAATTTCACAAGATAGT-3′ (SEQ ID NO: 106), 5′-TACAAGTGATGACCTTGATTT-3′ (SEQ ID NO: 107), 5′-ACTGATCAAGAACTAGATGAA-3′ (SEQ ID NO: 108), 5′-AAGAACTAGATGAAACTCTTA-3′ (SEQ ID NO: 109), 5′-CCCACCTTTAGATAAAGAGAA-3′ (SEQ ID NO: 110), 5′-CAGCACGATTTCTGTTTAGAT-3′ (SEQ ID NO: 111), 5′-AGCACGATTTCTGTTTAGATA-3′ (SEQ ID NO: 112), or 5′-TAGATAATACACCACTACATT-3′ (SEQ ID NO: 113). In another example, the nucleic acid has at least 60%, at least 65%, at least 70%, at least 75%, at least 80%, at least 85%, at least 90%, or at least 95% identity to a sequence that is, but not limited to 5′-CGTGGATTGAACTTACTAAAT-3′ (SEQ ID NO: 4), 5′-ATTTAGTAAGTTCAATCCACG-3′ (SEQ ID NO: 91), 5′-CCGCGAAGTTATGAGCAAGTT-3′ (SEQ ID NO: 5), 5′-AACTTGCTCATAACTTCGCGG-3′ (SEQ ID NO: 92), 5′-CGGCCATTTGAACGCAAATTT-3′ (SEQ ID NO: 6), 5′-AAATTTGCGTTCAAATGGCCG-3′ (SEQ ID NO: 93), 5′-GCAATGGTTAACGGAGTTTCA-3′ (SEQ ID NO: 104), 5′-AATGGTTAACGGAGTTTCAAT-3′ (SEQ ID NO: 105), 5′-AAGGAAATTTCACAAGATAGT-3′ (SEQ ID NO: 106), 5′-TACAAGTGATGACCTTGATTT-3′ (SEQ ID NO: 107), 5′-ACTGATCAAGAACTAGATGAA-3′ (SEQ ID NO: 108), 5′-AAGAACTAGATGAAACTCTTA-3′ (SEQ ID NO: 109), 5′-CCCACCTTTAGATAAAGAGAA-3′ (SEQ ID NO: 110), 5′-CAGCACGATTTCTGTTTAGAT-3′ (SEQ ID NO: 111), 5′-AGCACGATTTCTGTTTAGATA-3′ (SEQ ID NO: 112), or 5′-TAGATAATACACCACTACATT-3′ (SEQ ID NO: 113).

In another example, the nucleic acid encoding a Tjp1 inhibitor is, but not limited to i) a combination of SEQ ID NO: 4 and SEQ ID NO: 91, wherein SEQ ID NO: 4 is flanked at the 5′end by a nucleotide sequence comprising 1 to 10 nucleotides, wherein a nucleotide sequence of 1 to 20 nucleotides connects the 3′end of SEQ ID NO: 4 with the 5′end of SEQ ID NO: 91, and wherein SEQ ID NO: 91 is flanked at the 3′end by a nucleotide sequence comprising 1 to 10 nucleotides; ii) a combination of SEQ ID NO: 5 and SEQ ID NO: 92, wherein SEQ ID NO: 5 is flanked at the 5′end by a nucleotide sequence comprising 1 to 10 nucleotides, wherein a nucleotide sequence of 1 to 20 nucleotides connects the 3′end of SEQ ID NO: 5 with the 5′end of SEQ ID NO: 92, and wherein SEQ ID NO: 92 is flanked at the 3′end by a nucleotide sequence comprising 1 to 10 nucleotides; or iii) a combination of SEQ ID NO: 6 and SEQ ID NO: 93, wherein SEQ ID NO: 6 is flanked at the 5′end by a nucleotide sequence comprising 1 to 10 nucleotides, wherein a nucleotide sequence of 1 to 20 nucleotides connects the 3′end of SEQ ID NO: 6 with the 5′end of SEQ ID NO: 93, and wherein SEQ ID NO: 93 is flanked at the 3′end by a nucleotide sequence comprising 1 to 10 nucleotides. In another example, the nucleic acid encoding a Tjp1 inhibitor is, but not limited to at least 60%, at least 65%, at least 70%, at least 75%, at least 80%, at least 85%, at least 90%, or at least 95% identity to i) a combination of SEQ ID NO: 4 and SEQ ID NO: 91, wherein SEQ ID NO: 4 is flanked at the 5′end by a nucleotide sequence comprising 1 to 10 nucleotides, wherein a nucleotide sequence of 1 to 20 nucleotides connects the 3′end of SEQ ID NO: 4 with the 5′end of SEQ ID NO: 91, and wherein SEQ ID NO: 91 is flanked at the 3′end by a nucleotide sequence comprising 1 to 10 nucleotides; ii) a combination of SEQ ID NO: 5 and SEQ ID NO: 92, wherein SEQ ID NO: 5 is flanked at the 5′end by a nucleotide sequence comprising 1 to 10 nucleotides, wherein a nucleotide sequence of 1 to 20 nucleotides connects the 3′end of SEQ ID NO: 5 with the 5′end of SEQ ID NO: 92, and wherein SEQ ID NO: 92 is flanked at the 3′end by a nucleotide sequence comprising 1 to 10 nucleotides; or iii) a combination of SEQ ID NO: 6 and SEQ ID NO: 93, wherein SEQ ID NO: 6 is flanked at the 5′end by a nucleotide sequence comprising 1 to 10 nucleotides, wherein a nucleotide sequence of 1 to 20 nucleotides connects the 3′end of SEQ ID NO: 6 with the 5′end of SEQ ID NO: 93, and wherein SEQ ID NO: 93 is flanked at the 3′end by a nucleotide sequence comprising 1 to 10 nucleotides. In another example, SEQ ID NOs 4, 5 or 6 is flanked at the 5′end by a nucleotide sequence comprising 3 to 8 nucleotides, 4 to 6 nucleotides, or 1, 2, 3, 4, 5, 6, 7, 8, 9, or 10 nucleotides; a nucleotide sequence of 4 to 15 nucleotides, 8 to 13 nucleotides, or 1, 2, 3, 4, 5, 6, 7, 8, 9, 10, 11, 12, 13, 14, 15, 16, 17, 18, 19 or 20 nucleotides connect the 3′end of SEQ ID NO: 4, 5 or 6 with the 5′end of SEQ ID NO: 91, 92 or 93 respectively; and SEQ ID NOs 91, 92 or 93 is flanked at the 3′end by a nucleotide sequence comprising 3 to 8 nucleotides, 4 to 6 nucleotides, or 1, 2, 3, 4, 5, 6, 7, 8, 9, or 10 nucleotides. In yet another example, the nucleic acid encoding a Tjp1 inhibitor is, but not limited to i) a combination of SEQ ID NO: 4 and SEQ ID NO: 91, wherein SEQ ID NO: 4 is flanked at the 5′end by a nucleotide sequence comprising 4 nucleotides, wherein a nucleotide sequence of 6 nucleotides connects the 3′end of SEQ ID NO: 4 with the 5′end of SEQ ID NO: 91, and wherein SEQ ID NO: 91 is flanked at the 3′end by a nucleotide sequence comprising 6 nucleotides; ii) a combination of SEQ ID NO: 5 and SEQ ID NO: 92, wherein SEQ ID NO: 5 is flanked at the 5′end by a nucleotide sequence comprising 4 nucleotides, wherein a nucleotide sequence of 6 nucleotides connects the 3′end of SEQ ID NO: 5 with the 5′end of SEQ ID NO: 92, and wherein SEQ ID NO: 92 is flanked at the 3′end by a nucleotide sequence comprising 6 nucleotides; or iii) a combination of SEQ ID NO: 6 and SEQ ID NO: 93, wherein SEQ ID NO: 6 is flanked at the 5′end by a nucleotide sequence comprising 4 nucleotides, wherein a nucleotide sequence of 6 nucleotides connects the 3′end of SEQ ID NO: 6 with the 5′end of SEQ ID NO: 93, and wherein SEQ ID NO: 93 is flanked at the 3′end by a nucleotide sequence comprising 6 nucleotides.

In yet another example, the nucleic acid comprises a sequence that is, but not limited to 5′-CCGGCGTGGATTGAACTTACTAAATCTCGAGATTTAGTAAGTTCA ATCCACGTTTTTG-3′ (SEQ ID NO: 1), 5′-CCGGCCGCGAAGTTATGAGCAAGTTCTCGAGAACTTGCTCATAACTTCGCGGTTTTTG-3′ (SEQ ID NO 2), or 5′-CCGGCGGCCATTTGAACGCAAATTTCTCGAGAAATTTGCGTTCAAATGGCCGTTTTTG-3′ (SEQ ID NO: 3). In yet another example, the nucleic acid comprises a sequence of at least 60%, at least 65%, at least 70%, at least 75%, at least 80%, at least 85%, at least 90%, at least 95%, at least 96%, at least 97%, at least 98%, at least 99%, or 100% identity to a sequence that is, but not limited to 5′-CCGGCGTGGATTGAACTTACTAAATCTCGAGATTTAGTAAGTTCAATCCACGTTTTTG-3′ (SEQ ID NO: 1), 5′-CCGGCCGCGAAGTTATGAGCAAGTTCTCGAGAACTTGCTCATAACTTCGCGGTTTTTG-3′ (SEQ ID NO 2), or 5′-CCGGCGGCCATTTGAACGCAAATTTCTCGAGAAATTTGCGTTCAAATGGCCGTTTTTG-3′ (SEQ ID NO: 3).

The nucleic acid encoding a Tjp1 inhibitor can also be an siRNA. In one example, the nucleic acid encoding a Tjp1 inhibitor comprises a sequence that is, but not limited to 5′-UGAAACUCCGUUAACCAUUGC-3′ (SEQ ID NO: 94), 5′-AUUGAAACUCCGUUAACCAUU-3′ (SEQ ID NO: 95), 5′-ACUAUCUUGUGAAAUUUCCUU-3′ (SEQ ID NO: 96), 5′-AAAUCAAGGUCAUCACUUGUA-3′ (SEQ ID NO: 97), 5′-UUCAUCUAGUUCUUGAUCAGU-3′ (SEQ ID NO: 98), 5′-UAAGAGUUUCAUCUAGUUCUU-3′ (SEQ ID NO: 99), 5′-UUCUCUUUAUCUAAAGGUGGG-3′ (SEQ ID NO: 5′-100), AUCUAAACAGAAAUCGUGCUG-3′ (SEQ ID NO: 101), 5′-UAUCUAAACAGAAAUCGUGCU-3′ (SEQ ID NO: 102) and 5′-AAUGUAGUGGUGUAUUAUCUA-3′ (SEQ ID NO: 103). In one example, the nucleic acid encoding a Tjp1 inhibitor has at least 60%, at least 65%, at least 70%, at least 75%, at least 80%, at least 85%, at least 90%, at least 95%, at least 96%, at least 97%, at least 98%, at least 99%, or 100% identity to a sequence that is, but not limited to 5′-UGAAACUCCGUUAACCAUUGC-3′ (SEQ ID NO: 94), 5′-AUUGAAACUCCGUUAACCAUU-3′ (SEQ ID NO: 95), 5′-ACUAUCUUGUGAAAUUUCCUU-3′ (SEQ ID NO: 96), 5′-AAAUCAAGGUCAUCACUUGUA-3′ (SEQ ID NO: 97), 5′-UUCAUCUAGUUCUUGAUCAGU-3′ (SEQ ID NO: 98), 5′-UAAGAGUUUCAUCUAGUUCUU-3′ (SEQ ID NO: 99), 5′-UUCUCUUUAUCUAAAGGUGGG-3′ (SEQ ID NO: 100), 5′-AUCUAAACAGAAAUCGUGCUG-3′ (SEQ ID NO: 101), 5′-UAUCUAAACAGAAAUCGUGCU-3′ (SEQ ID NO: 102) and 5′-AAUGUAGUGGUGUAUUAUCUA-3′ (SEQ ID NO: 103).

Some of the nucleic acid sequences as disclosed herein are DNA sequences (such as any one of SEQ ID NOs: 1 to 6, 91 to 93 or 104 to 113, or combinations thereof). In one example, only one nucleic acid sequences as disclosed in any one or SEQ ID NOs: 1 to 6, 91 to 93 or 104 to 113 can be inserted into a single vector. In another example, more than one nucleic acid sequences as disclosed in any one or SEQ ID NOs: 1 to 6, 91 to 93 or 104 to 113 can be inserted into a single vector thereby a single vector will express more than one short hairpin molecules. Without wishing to be bound by theory, combination of multiple short hairpin molecules may increase the inhibitory effects of short hairpin molecules on Tjp1 expression or activity.

Other nucleic acid sequences as disclosed herein are RNA sequences, for example, siRNA sequences (such as any one of SEQ ID NOs: 94-103, or combinations thereof). Without wishing to be bound by theory, combination of siRNAs may increase the inhibitory effects of the siRNAs on Tjp1 expression or activity.

The Tjp1 inhibitor can be administered using any delivery system known in the art. The delivery method of the Tjp1 inhibitor includes, but is not limited to delivery method using virus-mediated delivery system. A person skilled in the art will appreciate that the list of viruses for the administration of the Tjp1 inhibitors via virus-mediated delivery system listed herein is not exhaustive. The examples of such viruses include, but are not limited to a retrovirus, an adenovirus, an adeno-associated virus, a herpes simplex virus, and the like. In one example, wherein when the delivery system of the Tjp1 inhibitor is virus-mediated delivery system, the virus includes, but is not limited to, a retrovirus, an adenovirus, an adeno-associated virus, a herpes simplex virus, and the like. In one example, wherein when the delivery system of the Tjp1 inhibitor is virus-mediated delivery system, the virus is an adeno-associated virus (AAV). The adeno-associated virus that can be used for the delivery of Tjp1 inhibitor has a variety of serotype. In one example, the adeno-associated virus that is used for the delivery of Tjp1 inhibitor includes, but is not limited to AAV serotype 1, AAV serotype 2, AAV serotype 3, AAV serotype 4, AAV serotype 5, AAV serotype 6, AAV serotype 7, AAV serotype 8, AAV serotype 9, AAV serotype 10, AAV serotype 11, and the like. In one example, the adeno-associated virus that is used for the delivery of Tjp1 inhibitor is AAV serotype 8.

In one example, wherein when the delivery system of the Tjp1 inhibitor is virus-mediated delivery system, the Tjp1 inhibitor comprises about 1×10⁷-1×10¹⁶ AAV, or about 1×10⁷-1×10⁹ AAV, about 1×10⁹-1×10¹¹ AAV, about 1×10¹¹-1×10¹³ AAV, about 1×10¹³-1×10¹⁵ AAV, about 1×10¹⁴-1×10¹⁶ AAV, or about 1×10⁷ AAV, about 2×10⁷ AAV, about 3×10⁷ AAV, about 4×10⁷ AAV, about 5×10⁷ AAV, about 6×10⁷ AAV, about 7×10⁷ AAV, about 8×10⁷ AAV, about 9×10⁷ AAV, about 1×10⁸ AAV, about 2×10⁸ AAV, about 3×10⁸ AAV, about 4×10⁸ AAV, about 5×10⁸ AAV, about 6×10⁸ AAV, about 7×10⁸ AAV, about 8×10⁸ AAV, about 9×10⁸ AAV, about 1×10⁹ AAV, about 2×10⁹ AAV, about 3×10⁹ AAV, about 4×10⁹ AAV, about 5×10⁹ AAV, about 6×10⁹ AAV, about 7×10⁹ AAV, about 8×10⁹ AAV, about 9×10⁹ AAV, about 1×10¹⁰ AAV, about 2×10¹⁰ AAV, about 3×10¹⁰ AAV, about 4×10¹⁰ AAV, about 5×10¹⁰ AAV, about 6×10¹⁰ AAV, about 7×10¹⁰ AAV, about 8×10¹⁰ AAV, about 9×10¹⁰ AAV, about 1×10¹¹ AAV, about 2×10¹¹ AAV, about 3×10¹¹ AAV, about 4×10¹¹ AAV, about 5×10¹¹ AAV, about 6×10¹¹ AAV, about 7×10¹¹ AAV, about 8×10¹¹ AAV, about 9×10¹¹ AAV, about 1×10¹² AAV, about 2×10¹² AAV, about 3×10¹² AAV, about 4×10¹² AAV, about 5×10¹² AAV, about 6×10¹² AAV, about 7×10¹² AAV, about 8×10¹² AAV, about 9×10¹² AAV, about 1×10¹³ AAV, about 2×10¹³ AAV, about 3×10¹³ AAV, about 4×10¹³ AAV, about 5×10¹³ AAV, about 6×10¹³ AAV, about 7×10¹³ AAV, about 8×10¹³ AAV, about 9×10¹³ AAV, about 1×10¹⁴ AAV, about 2×10¹⁴ AAV, about 3×10¹⁴ AAV, about 4×10¹⁴ AAV, about 5×10¹⁴ AAV, about 6×10¹⁴ AAV, about 7×10¹⁴ AAV, about 8×10¹⁴ AAV, about 9×10¹⁴ AAV, about 1×10¹⁵ AAV, about 2×10¹⁵ AAV, about 3×10¹⁵ AAV, about 4×10¹⁵ AAV, about 5×10¹⁵ AAV, about 6×10¹⁵ AAV, about 7×10¹⁵ AAV, about 8×10¹⁵ AAV, about 9×10¹⁵ AAV, or about 1×10¹⁶ AAV.

In another example, the nucleic acid sequences as disclosed herein (such as any one of SEQ ID NOs: 1 to 6 or 91 to 113), in full or fragments thereof, can be delivered as single strand nucleic acids (e.g. as siRNAs, antisense oligonucleotides, etc), either individually or in combinations thereof or as a DNA. These nucleic acids can be DNA (comprising thymidine) or RNA (comprising uracil) or even contain non-natural nucleotides that can bind the target. These nucleotides can also comprise additional modifications to enhance their stability. They can also be coupled to, for example, a N-acetylgalactosamine (GalNAc)-conjugation delivery system for nucleic acid.

The present invention provides Tjp1 inhibitor for use in therapy. In another example, the present invention provides a Tjp1 inhibitor for use in treating a liver disease. In yet another example, the present invention provides use of a Tjp1 inhibitor in the manufacture of a medicament for treating a liver disease. The term “liver disease” as used herein refers to abnormalities in liver structure and/or function. The expression disease or disorder can be used interchangeably. The abnormalities can occur to any structure that is found in the liver, including but not limited to cells such as hepatocytes, blood vessels in the liver or bile duct. These abnormalities can occur spontaneously, or be induced by non-liver cells, for example but not limited to immune cell infiltration mediated inflammation, or be induced by toxins, chemicals or drugs. The liver forms part of the biliary system, wherein the liver, bile duct and gall bladder work together to produce, store, secrete, and transport bile. Due to the close relationship of the organs in the biliary system, it would be appreciated that any abnormality in the biliary system can cause a liver disorder. Examples of liver diseases include, but are not limited to cholestasis, liver cancer, alcoholic liver disease, nonalcoholic steatohepatitis (NASH), nonalcoholic fatty liver disease (NAFLD), cholestatic liver disease, hepatitis A, hepatitis B, hepatitis C, hepatitis D, hepatitis E, liver fibrosis, liver cirrhosis, cholestasis-related progressive bile duct injury, cystic fibrosis-associated liver disease, thioacetamide (TAA)-related liver disease, 3,5-Diethoxycarbonyl-1,4-Dihydrocollidine (DDC)-related liver disease, bile duct ligation liver injury, Yes-associated Protein (YAP)-related liver disease, Mdr2-related liver disease, a disease related to the exposure to medications that affect cholesterol/bile acid (BA) biosynthesis and/or metabolism, a disease related to genetic mutations that affect cholesterol/bile acid (BA) biosynthesis and/or metabolism, or a disease related to the compromise of the integrity the bile blood barrier.

The terms “cholestasis” and “cholestatic liver disease” as used herein refer to a decrease in bile flow from the liver to the gall bladder due to impaired secretion by hepatocytes, to obstruction of bile flow through intrahepatic or extrahepatic bile ducts or as a consequence of liver damage (to hepatocytes or bile duct epithelial cells) caused by other diseases or injury in the liver. Many liver diseases have been shown to eventually lead to cholestasis. This results in the retention of bile salts in the liver which, under normal conditions, are excreted into bile. Examples of cholestasis include, but are not limited to intrahepatic cholestasis, primary biliary cholangitis (PBC), primary sclerosing cholangitis (PSC), pregnancy-related intrahepatic cholestasis, neonatal cholestasis, progressive familial intrahepatic cholestasis type 3, cholestatic fibrosis or biliary atresia. Causes of intrahepatic cholestasis include immune-mediated conditions such as, but not limited to primary biliary cholangitis (PBC), primary sclerosing cholangitis (PSC), intrahepatic cholestasis of pregnancy, progressive familial intrahepatic cholestasis type 3, and cystic fibrosis-associated liver disease, exposure to medications (steroids, nonsteroidal anti-inflammatory drugs, antibiotics, anti-diabetic agents) and genetic mutations (including inactivation of TJP2 or Δ4-3-oxosteroid 5β-reductase deficiency) that affect cholesterol/bile acid (BA) biosynthesis and/or metabolism or integrity the bile blood barrier. Cholestasis causes progressive bile duct injury, resulting in further retention of toxic hydrophobic bile acids. This leads to persistent and extensive damage to the bile duct, and ultimately causes damage to the liver.

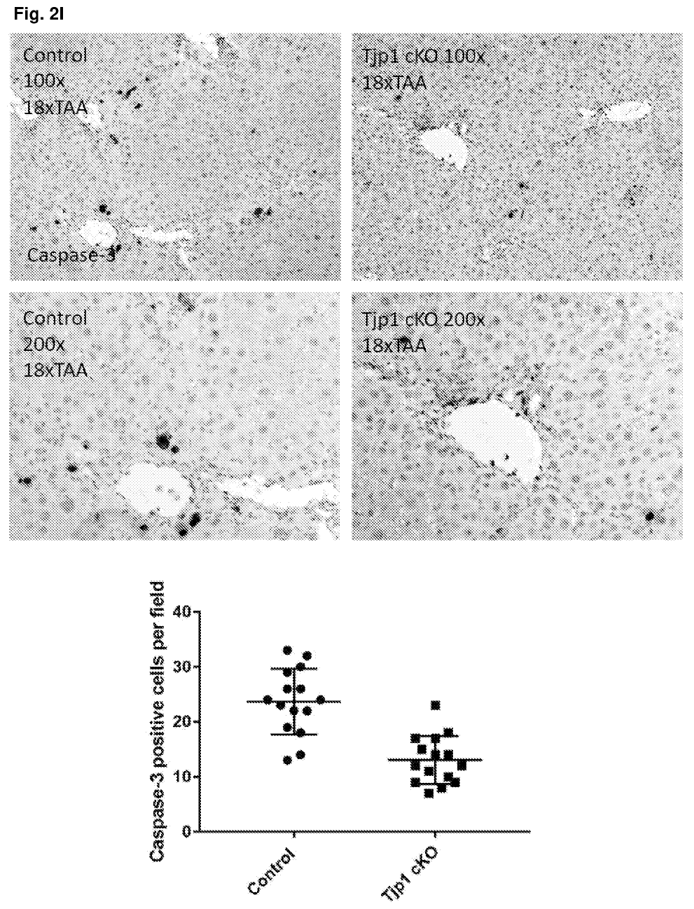

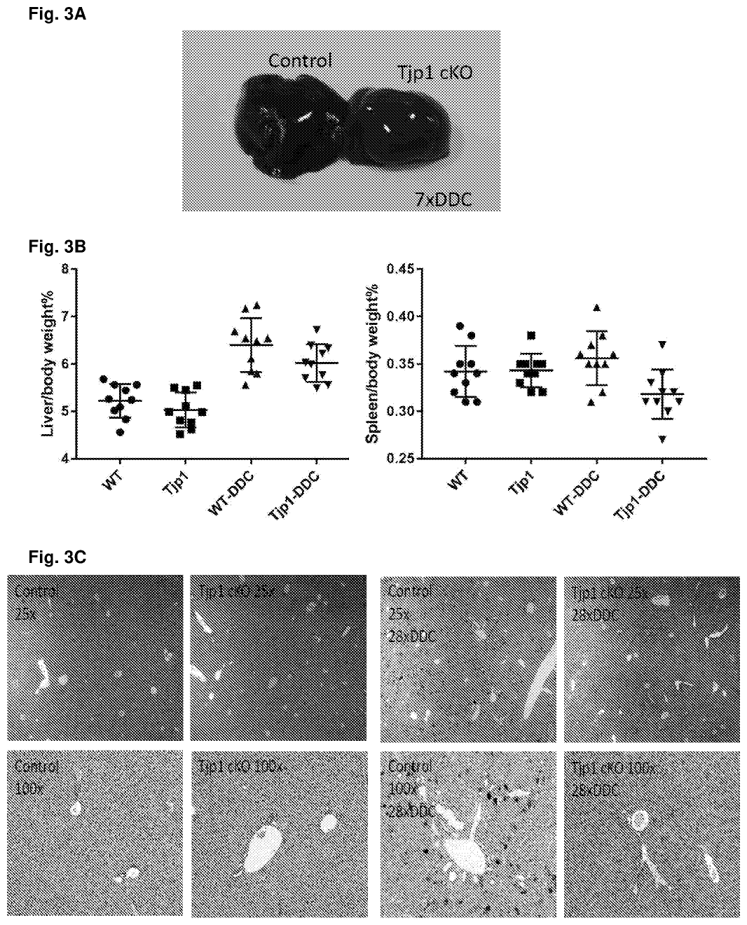

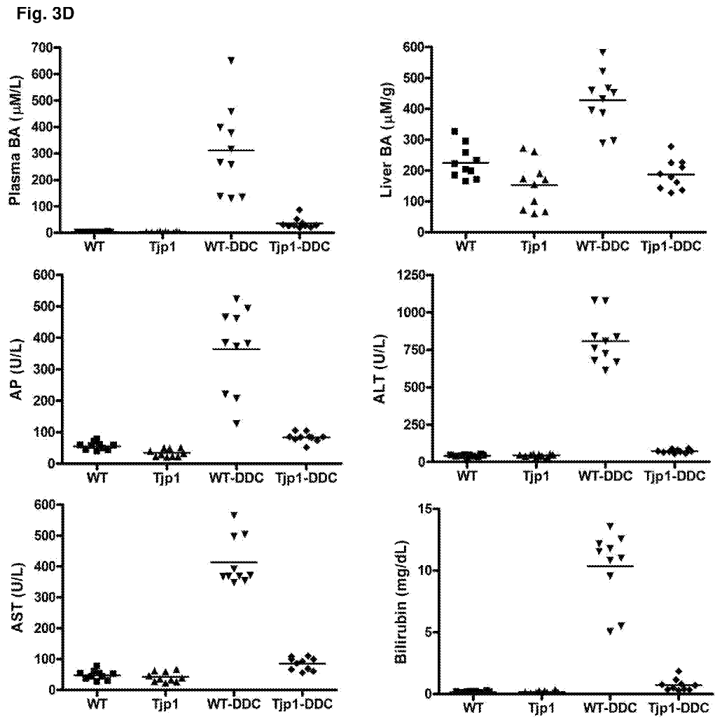

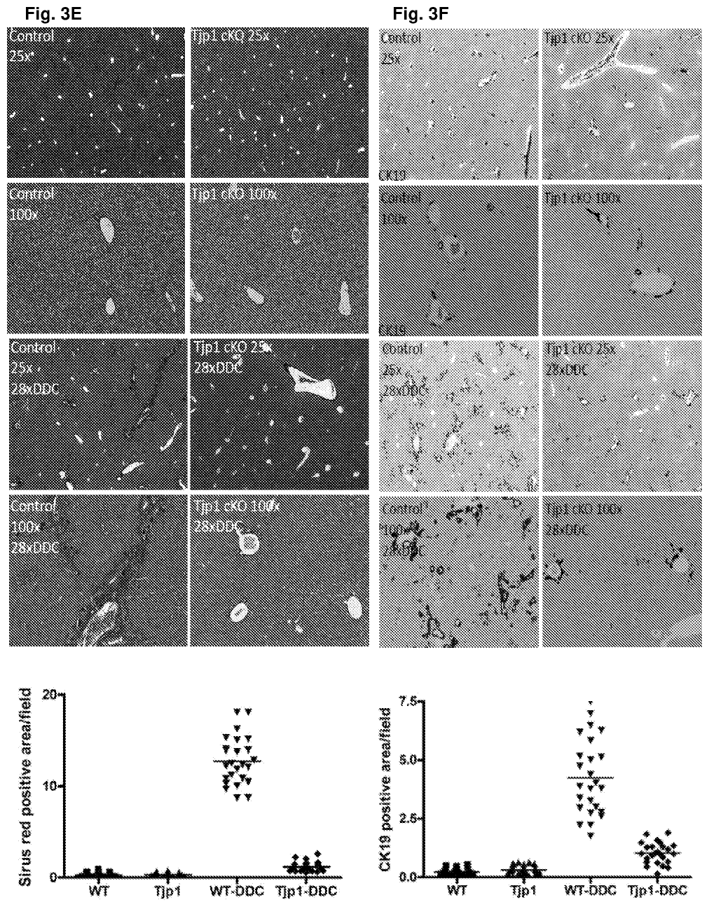

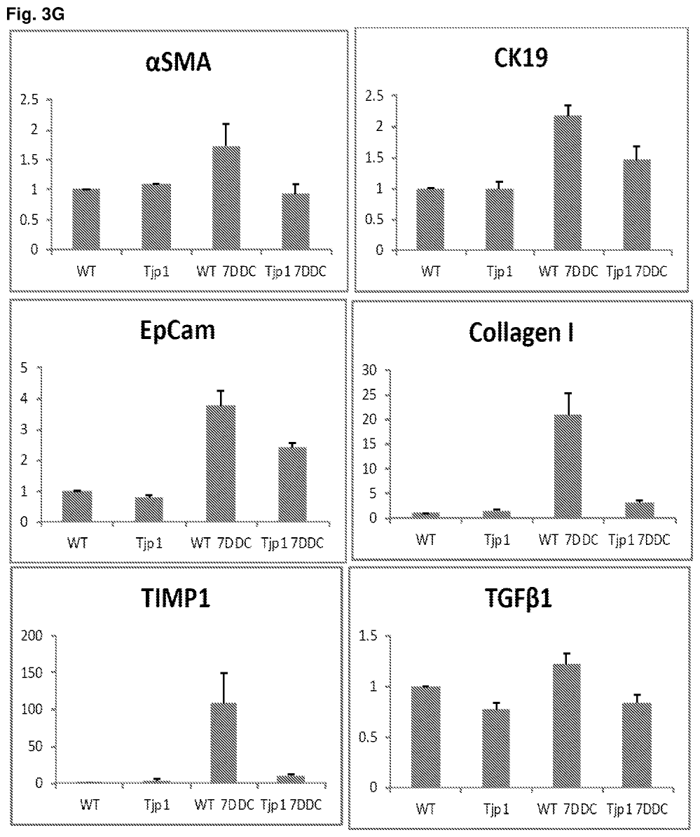

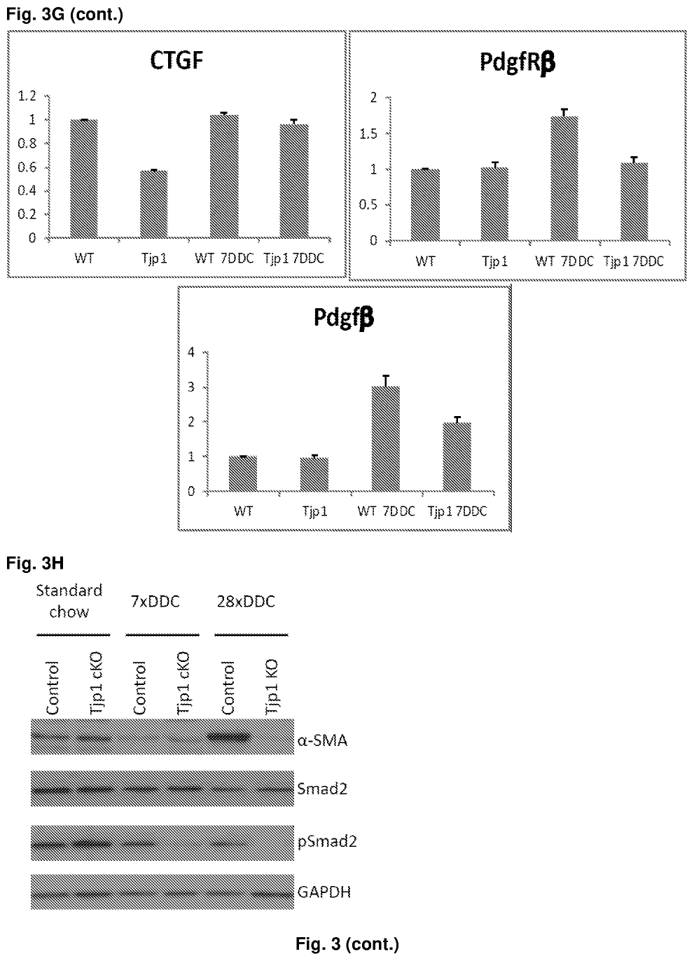

Some of the liver diseases can result in the activation of inflammatory processes such as, but not limited to, immune cell infiltration, increased expression of inflammatory cytokines and/or chemokines. Long term inflammatory response can cause liver fibrosis, which can lead to liver cirrhosis. The liver diseases can also cause necrosis of cells in the liver. Tjp1 inhibition can reduce the onset and progression of these effects. For example, Tjp1 inhibition can reduce the levels of TAA-induced liver necrosis (FIG. 2D), liver fibrosis (FIGS. 2F-2G, 3E, 4C, 5B), and inflammation and immune cell infiltration in the liver (FIG. 3K, 4D)

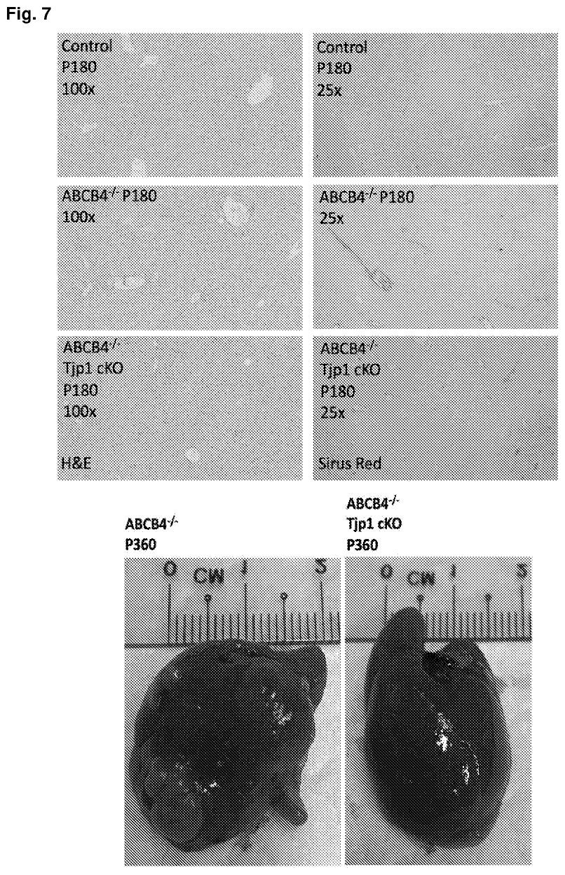

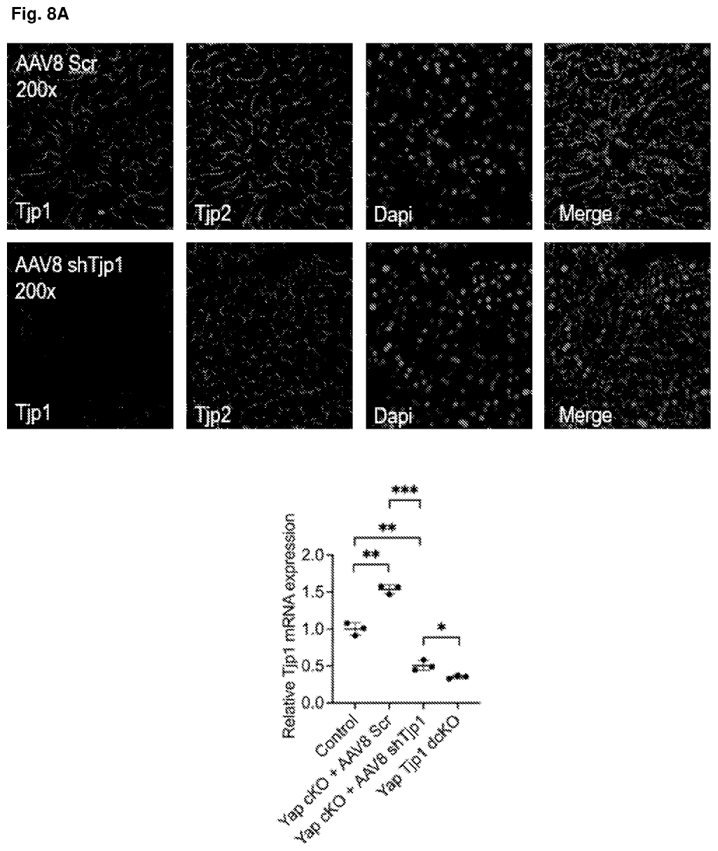

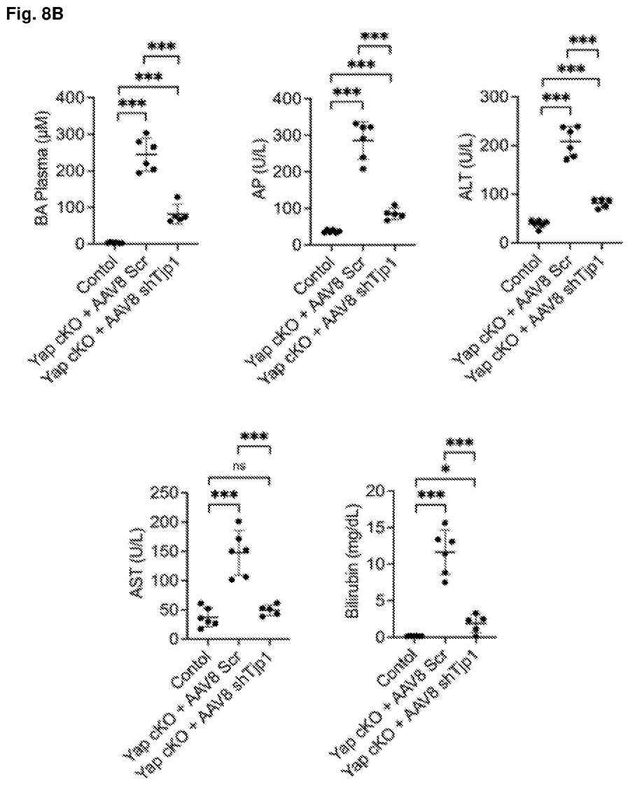

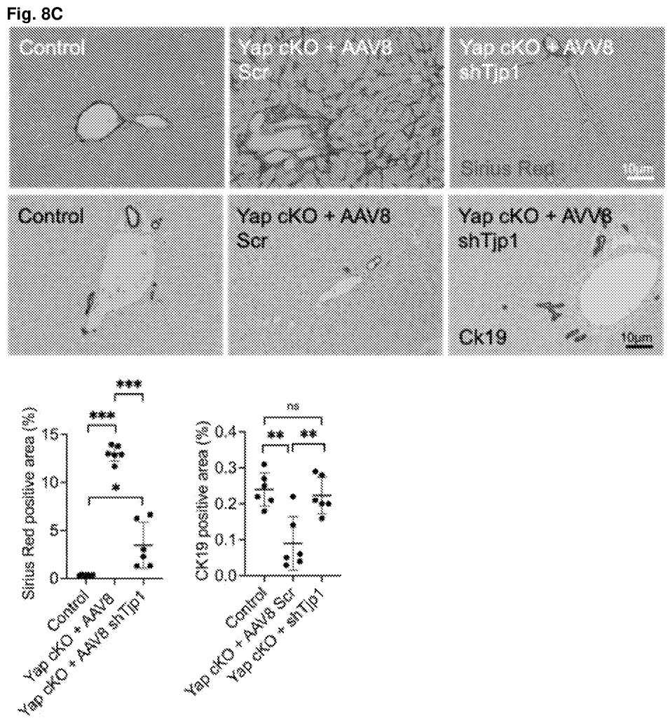

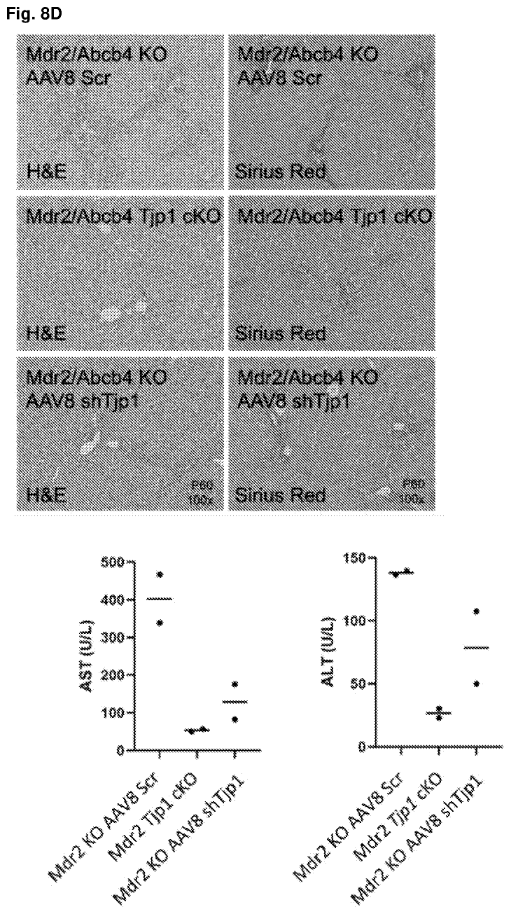

Tjp1 inhibition also provides a protective role in liver cancer, as shown by reduced levels of liver fibrosis in Mdr2 KO mice lacking Tjp1 (FIG. 7). Liver cancer can occur in livers damaged by genetic defects, alcohol abuse, or chronic infection with diseases such as hepatitis B and C. Chronic cholestasis, liver fibrosis and inflammation are also known to predispose a subject to liver cancer. In one example, liver cancer can be, but is not limited to hepatocellular carcinoma, cholangiocarcinoma, hepatoblastoma, or metastatic liver cancer.

This concomitant inactivation of Tjp1 resulted in the regeneration of a biliary system (FIG. 5G). Thus, in another aspect, the present invention provides a method for regenerating a biliary system in a subject, wherein the method comprises administering of a pharmaceutically effective amount of Tjp1 inhibitor to the subject. The terms “biliary system”, “biliary tract” and “biliary tree” as used herein refer to the ducts and organs that function to produce, store, secrete, and transport bile. The organs of the biliary system include, but are not limited to the liver, bile ducts (intra-hepatic and/or extrahepatic) and gall bladder. The biliary system is part of the liver, therefore any biliary disease would also be recognized to be a liver disease. In another example, the present invention provides a Tjp1 inhibitor for use in regenerating a biliary system. In yet another example, the present invention provides use of a Tjp1 inhibitor in the manufacture of a medicament for regenerating a biliary system.

In another aspect, the present invention provides a kit comprising a Tjp1 inhibitor as described herein and/or the nucleic acid as described herein. In one example, the present invention provides a pharmaceutical composition comprising a Tjp1 inhibitor as described herein. In yet another example, the pharmaceutical composition further comprises one or more pharmaceutically acceptable excipients, vehicles or carriers. Therefore, in one example, the pharmaceutical composition comprising the Tjp1 inhibitor as disclosed herein may further comprise a compound selected from, but not limited to, a pharmaceutically acceptable carrier, a liposomal carrier, an excipient, an adjuvant or combinations thereof.

As used in this application, the singular form “a,” “an,” and “the” include plural references unless the context clearly dictates otherwise. For example, the term “a genetic marker” includes a plurality of genetic markers, including mixtures and combinations thereof.

As used herein, the terms “increase” and “decrease” refer to the relative alteration of a chosen trait or characteristic in a subset of a population in comparison to the same trait or characteristic as present in the whole population. An increase thus indicates a change on a positive scale, whereas a decrease indicates a change on a negative scale. The term “change”, as used herein, also refers to the difference between a chosen trait or characteristic of an isolated population subset in comparison to the same trait or characteristic in the population as a whole. However, this term is without valuation of the difference seen.

As used herein, the term “about” in the context of concentration of a substance, size of a substance, length of time, or other stated values means +/−5% of the stated value, or +/−4% of the stated value, or +/−3% of the stated value, or +/−2% of the stated value, or +/−1% of the stated value, or +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### EXPERIMENTAL SECTION

### Material and Methods

### Mouse Disease Models

To characterize the role of Tjp1/ZO-1 in the liver, Tjp1 was inactivated in a liver specific manner in different mouse models. Albumin (Alb)-Cre driver lines were used to achieve deletion of Tjp1 in early liver development, both in hepatocytes and cholangiocytes, in Tjp1 conditional knockout (cKO) mice. Alb-CreERT2 or Sox9-CreERT2 lines were used to achieve tamoxifen inducible Tjp1 conditional knockout (Tjp1 icKO). This inducible Tjp1 conditional knockout system can be applied specifically to hepatocytes or cholangiocytes to obtain mice with inducible Tjp1 conditional knockout hepatocytes (Tjp1 icKOHC mice) or cholangiocytes (Tjp1 icKOCC mice).

### Experimental Assays

Different assays were established, including plasma and liver biochemistry, to monitor liver function and histology, as well as immunohistochemical assays to assess tissue changes and fibrosis. Immunostaining for different markers was used to assess changes at the cellular level. EM and tracer permeability assays in vivo were used to assess the tight junction barrier. To assess if deletion of the Tjp1 had an effect on disease susceptibility, several liver disease models were established, including feeding of diets supplemented with thioacetamide (TAA), 3,5-diethoxycarbonyl-1,4-dihydrocollidine (DDC), bile duct ligation (BDL), or crossing to other liver disease models such as the Mdr2 KO and Yap cKO mice.

The effect of the absence of Tjp1/ZO-1 in cholangiocytes and/or hepatocytes was assessed using standard biochemical assays for liver injury and function (e.g. blood AST, ALT and bilirubin levels, blood and liver bile acid (BA) levels). Histology, immunohistochemisrty, immunofluorescence microscopy and Western blot analysis were use to identify hepatocytes and cholangiocytes, assess liver fibrosis (Sirius red, collagen, α-smooth muscle actin), monitor the expression levels and localization of hepatocyte and cholengiocyte markers of interest, and assess immune cell infiltration. Quantitative RT-PCR was carried out to determine changes in liver detoxifying enzyme and bile acid transporter expression in response to Tjp1 inactivation.

### Mouse Strains, Genotyping

Animal experimentation was approved by the relevant IACUC (Protocol #171211 and #201558) under SPF conditions. Tjp1 was inactivated by crossing C57BL/6Tac Tjp 1^{F/F} mice to Alb-Cre mice for constitutive deletion in hepatocytes and cholangiocytes (Tjp1 cKO). The Alb-Cre and the Rosa26: LacZ (Rosa26: Lox-STOP-Lox-LacZ) reporter line (B6; 129S4-Gt (ROSA) 26Sortm1Sor/J)³², used for lineage tracing, were from Jackson Laboratory. Mice were backcrossed into the Tjp1^{F/F} background. For genotyping, genomic DNA isolated from tail clippings was amplified using primer-1 (5′-CTT CTC TGA CCC TAC ACA GOT ACC-3′) (SEQ ID NO: 13) and primer-2 (5′-ATC GTG TGG GAA AGA CAA GC-3′) (SEQ ID NO: 14), yielding a 279 bp (wild-type allele) or a 471 bp (conditional mutant allele) fragment. C57BL/6Tac Tjp1^{F/F} littermates served as controls. Tjp1^{F/F} and Alb-Cre mice were crossed with Yap^{F/F} mice²³ to generate Tjp1 Yap conditional knockout (cKO) mice. Male animals were used, but female mice showed comparable results. For genotyping, genomic DNA isolated from tail clippings was amplified using Yap specific primer 1 (5′-CCA TTT GTC CTC ATC TCT TAC TAA C-3′) (SEQ ID NO: 15) and primer 2 (5′-GAT TGG GCA CTG TCA ATT AAT GGG CTT-3′) (SEQ ID NO: 16), yielding a 498 bps (wild type allele) or a 597 bps conditional mutant allele) fragment. Abcb4/Mdr2 (FVB.129P2-Abcb4^{tm1Bor}/J) (Jackson Laboratory) were crossed with Tjp1^{F/F} animals to delete Tjp1.

### Thioacetamice (TAA) Treatment

Thioacetamice (TAA) (Cat #163678, Sigma) was dissolved in PBS and intraperitoneally injected into the mice at 200 mg/kg. For acute liver injury, the mice were injected with TAA and liver samples were collected 6, 24, and 48 hours later. For chronic liver injury, the mice were injected with TAA every two days, 3 times per week for 6 weeks. After the 18th injection, the liver samples were collected for analysis.

### DDC-Diet

Mice were fed with chow diet supplemented with 0.1% 3,5-Diethoxycarbonyl-1,4-Dihydrocollidine (DDC: Cat #D80002, Sigma) for 28 days. When treated with tamoxifen, mice were provided with the DDC-diet one week after the last tamoxifen dose.

### Bile Duct Ligation

8-10 week old mice were anesthetized and their common bile ducts were ligated. The mice with ligated common bile ducts were kept for 7 days and then sacrificed and liver samples were collected for analysis. To make sure the ligation was tight, only those mice with more than 100 μl bile in their gallbladder were used.

### Assessment of Hepatic Barrier Function

FITC-Dextran 4 kDa (MW 4000, Sigma-Aldrich) was dissolved in PBS (25 mg/ml). Mice received 100 μl of the tracer solution via tail-vein injection and were. sacrificed 8 minutes later. The bile was collected from gallbladder and FITC-fluorescence measured in a plate reader (Tecan). To disrupt hepatic tight junctions (TJs), LPS (2 mg/kg) was injected into control mice 16 hours before tracer injection. The number of mice per cohort analyzed are given in the figure legend and samples were analyzed in 2-3 different and independent experimental runs.

### Serum and Tissue Biochemical Analyses

Kits were used to determine bilirubin, serum alanine aminotransferase (ALT), alkaline phosphatase (AP) and aspartate aminotransferase (AST) (Teco Diagnostics), or plasma total BA (Diazyme Laboratories) levels. For liver BA levels, 100 mg liver tissue was ground in liquid nitrogen, suspended in 1 mL water, sonicated, centrifuged, and BA levels were determined in the supernatant. The numbers of mice per cohort analysed are given in the respective figure legends, and samples were analysed in 2 or 3 different and independent experimental runs.

### Electron Microscopy

Mouse livers were perfused with 2.5% glutaraldehyde fixative via the inferior vena cava, cut into small pieces and fixed for 24 hours. Samples were, rinsed with PBS, post-fixed (1% osmium tetroxide, 1h), rinsed, dehydrated in ethanol, and embedded in resin. Ultrathin sections were stained with uranyl acetate and lead citrate, and viewed with a transmission EM (JEM-1010). For morphometric analysis, the length and width of the tight junction (TJ) plaque were measured. For width, the distance between two bordering cells was measured along the TJ plaque at five different locations and the average used as the width of that particular TJ. Microvilli protruding into the canaliculus were counted and normalized to circumference unit. To account for possible differences due to location in the tissue, blocks from both the edge and center of liver lobes (4 blocks for each animal) were sectioned. Since the measurements are also influenced by the plane of the section, each block was sectioned from different angles to normalize for these differences.

### Histology

Freshly dissected livers were fixed in 4% paraformaldehyde overnight, processed, and embedded in paraffin. 5 μm sections were stained with H&E or Sirius red and imaged with a Zeisscam camera on a Zeiss Axio microscope. Five mice per cohort and images from at least 2 slides for each mouse were analyzed and used for quantification.

### Immunohistochemistry

Paraffin blocks were sectioned at a thickness of 5 μm. For immunohistochemistry, antigens were retrieved by steaming the slides for 20 min in a 2100 Retriever (Pick Cell Laboratories). The slides were then stained with primary antibodies against cleaved caspase 3 (rabbit, Cat #9661, Cell Signaling), Laminin1-2 (rabbit; Cat #ab11575, Abcam), Collagen (rabbit; Cat #NB600-408, NOVUS Biologicals), F4/80 (rat; Cat #600-404, NOVUS Biologicals), CD11b (rat; Cat #ab8878, Abcam), Ki67 (rabbit; Cat #9129 Cell Signaling), Ck19 (rat; Troma III, DSHB, 1:20 dilution), and compatible biotin conjugated secondary antibodies (Invitrogen).

### Immunofluorescence Microscopy

Paraffin sections were de-waxed and antigens retrieved by steaming for 20 min (2100 Retriever; Pick Cell Laboratories). Frozen tissues were embedded in OCT and 5 μm sections stained with primary antibodies against ZO-1 (rat; Cat #R26.4C DSHB), Claudin-1 (rabbit; Cat #71-7800 Invitrogen), Claudin-2 (rabbit; Cat #32-5600 Invitrogen), Claudin-3 (rabbit; Cat #34-1700 Invitrogen), Cingulin (rabbit; Cat #36-4401 Invitrogen), CK19 (rat; Cat #TromaIII DSHB), DPPIV (goat; Cat #AF954 R&D and compatible fluorescently labeled secondary antibodies (Invitrogen, 1:200 dilution). Nuclei were labeled with DAPI. Images were obtained using Zeiss LSM800 confocal microscope. Three mice per cohort and at least 5 sections for each mouse were analyzed for quantification.

### Edu Labelling

5-ethynyl-2′-deoxyuridine (Edu) is dissolved in DMSO (1 mg/10 μl) and further diluted in PBS (1 mg/100 μl). This solution was injected intraperitoneally (1 mg/g body weight) 1 hour before sacrificing the mice. Images were taken from at least 3 independent mice using a Zeiss LSM800 confocal microscope and Zen v3.4 (blue edition) software.

### LacZ Staining

Livers were dissected seven days after the last tamoxifen injection, frozen in optimal cutting temperature compound (OCT), and 10 μm-thick sections cut and mounted on slides. After fixation in formalin for 10 minutes, LacZ staining was carried out (NovaUltra kit) per the manufacturer's protocol. Slides were counterstained with Nuclear Faster Red for 3-5 minutes. Three mice per cohort and at least 5 sections for each mouse were analysed.

### Western Blotting

Fresh liver samples were frozen in liquid nitrogen, crushed into powder and lysed for 15 min on ice in lysis buffer (50-mM Tris-HCl, pH7.5, 100 mM NaCl, 1 mM MgCl₂, and 0.5% Triton X-100, supplemented with protease inhibitor cocktail and one PhosSTOP tablet per 10 ml [Cat. #04 906 837 01, Roche]). Lysates were sonicated and centrifuged (13,000×g for 15 min) at 4° C. Supernatants were collected and equal amounts of protein were fractionated by SDS-polyacrylamide gel electrophoresis and subjected to Western blotting using antibodies against Claudin-1 (rabbit; Cat #ab15098, Abcam), Claudin-2 (rabbit; Cat #32-5600 Invitrogen), Claudin-3 (rabbit; Cat #34-1700, Invitrogen), Occludin (rabbit; Cat #71-1500, Invitrogen), Cingulin (rabbit; Cat #36-4401 Invitrogen), E-cadherin (mouse; Cat #610181, BD Biosciences), Smad2 (rabbit; Cat #5339, Cell Signaling), Phospho-Smad2 (rabbit; Cat #3108, Cell Signaling), Laminin1-2 (rabbit; Cat #ab11575, Abcam), aSMA (rabbit; Cat #ab5694, Abcam), Timp-1 (rabbit; Cat #ab211926, Abcam), ICAM-1 (mouse; Cat #MA5406, ThermoFisher), Osteopontin (mouse; Cat #MA5-17180), Vinculin (mouse; Cat #V9131, Sigma), or GAPDH (Mouse; Cat #sc-47724, Santa Cruz). Vinculin or GAPDH served as loading control. Primary and secondary antibodies were diluted 1:1000 and 1:3000, respectively. Samples from at least 3 independent mice per cohort were analyzed using ImageJ v15.3.

mRNA Extraction and Real-Time PCR

Total messenger RNA (mRNA) was extracted from whole liver and processed for quantitative reverse-transcription polymerase chain reaction using a QuantStudioTM 3 Real-Time PCR System (Applied Biosystems) and specific primers (SEQ ID NOs: 17-90). mRNA expression levels were normalized to GAPDH. Samples were isolated from at least 3 mice per cohort, pooled and run in triplicates. Each such experiment was independently repeated three times and all the data were combined for analyses.

AAV8 vector with DNA encoding Tjp1 shRNA mediating gene silencing

Three different DNAS that encode shTjp1 shRNAs (5′-CCGGCGTGGATTGAACTTACTAAATCTCGAGATTTAGTAAGTTCAATCCACGTTTTTG-3′ (SEQ ID NO: 1), 5′-CCGGCCGCGAAGTTATGAGCAAGTTCTCGAGAACTTGCTCATAACTTCGCGGTTTTTG-3′ (SEQ ID NO: 2), and 5′-CCGGCGGCCATTTGAACGCAAATTTCTCGAGAAATTTGCGTTCAAATGGCCGTTTTTG-3′ (SEQ ID NO: 3) were generated. The DNAs encoding scrambled shRNA or one of the shTjp1 shRNAs were injected into 2-month old Mdr2/ABCB4 KO mice via orbital injection (5×10¹⁰ to 2.5×10¹⁰ virus particles per mouse). These mice were sacrificed 2, 4 or 6 months after injection. Liver samples were collected for analysis. Sequences mediating the interaction with the target, Tjp1, are in bold.

### Experimental Results

### Tjp1 is not Required for Liver Development and Dispensable for Liver Structure and Function

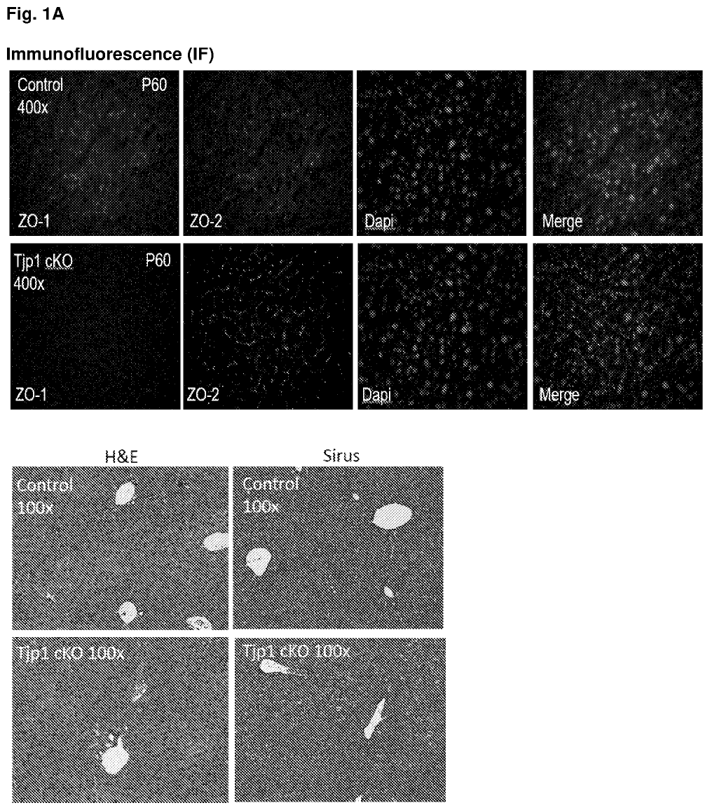

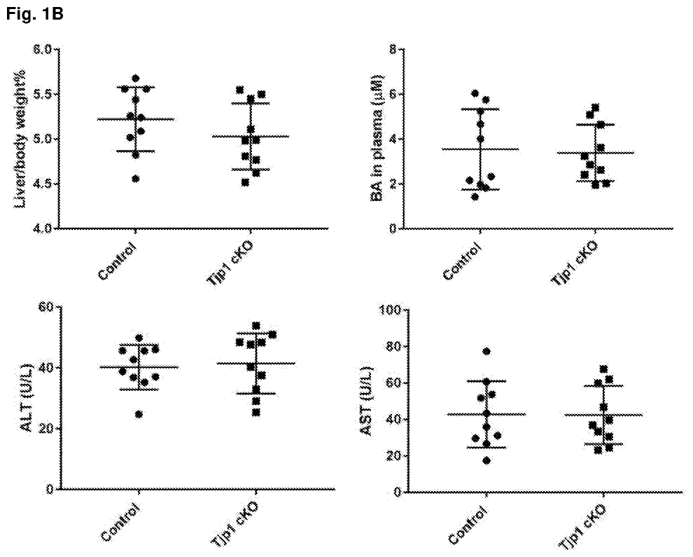

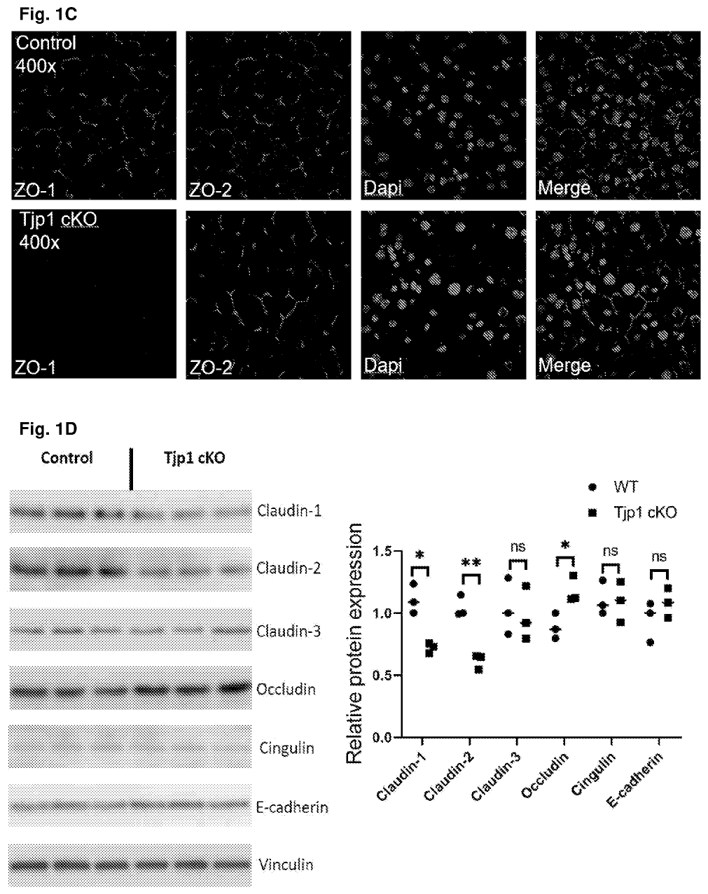

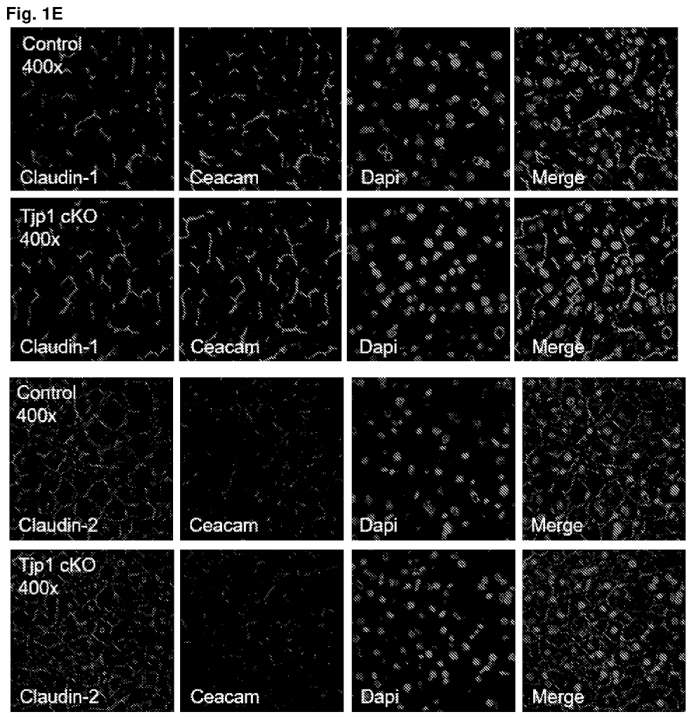

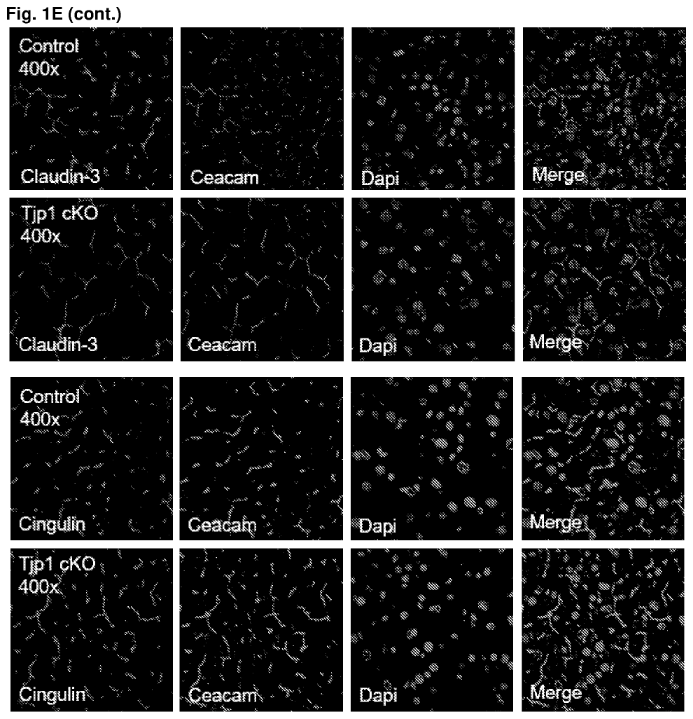

The albumin promoter is activated during liver development in common precursors that later give rise to both cholangiocytes and hepatocytes, resulting in floxed or deleted genes in both cell types of the Alb-Cre mice. In Tjp1 cKO mice, Tjp1 is inactivated and therefore absent from hepatocytes and cholangiocytes of the adult organ. This did not result in an overt phenotype with regards to liver function (FIG. 1B), liver histology (FIG. 1A), tight junction marker expression and localization (FIGS. 1A, C, and E), or tight junction structure (FIG. 1F) or tight junction function, which is assessed by in vivo permeability of 4 kDa FITC-dextran (FIG. 1G).

### Inactivation of Tjp1 is Protective in Several Liver Disease Models

Given the absence of an overt phenotype after liver-specific inactivation of Tjp1, the effect the absence of Tjp1 was assessed in different liver disease models using thioacetamide (TAA), 3,5-Diethoxycarbonyl-1,4-Dihydrocollidine (DDC) diet and bile duct ligation (BDL). TAA induced liver injury is commonly used to obtain liver fibrosis model. DDC diet induced liver injury and bile duct ligation are commonly used to achieve cholestatic disease (choleostasis) models. In addition, Yap cKO and Mdr2 knockout (KO) mice were used. Yap cKO mice have defective bile duct formation during liver development and is a good model for biliary disease. Mdr2 KO mice are considered to be good animal models for bile acid induced liver diseases in humans with defects in Mdr3, which is the human homolog of mouse Mdr2. Examples of such bile acid induced liver diseases include but are not limited to chronic inflammatory biliary liver disease, liver fibrosis and liver cirrhosis or primary sclerosing cholangitis.

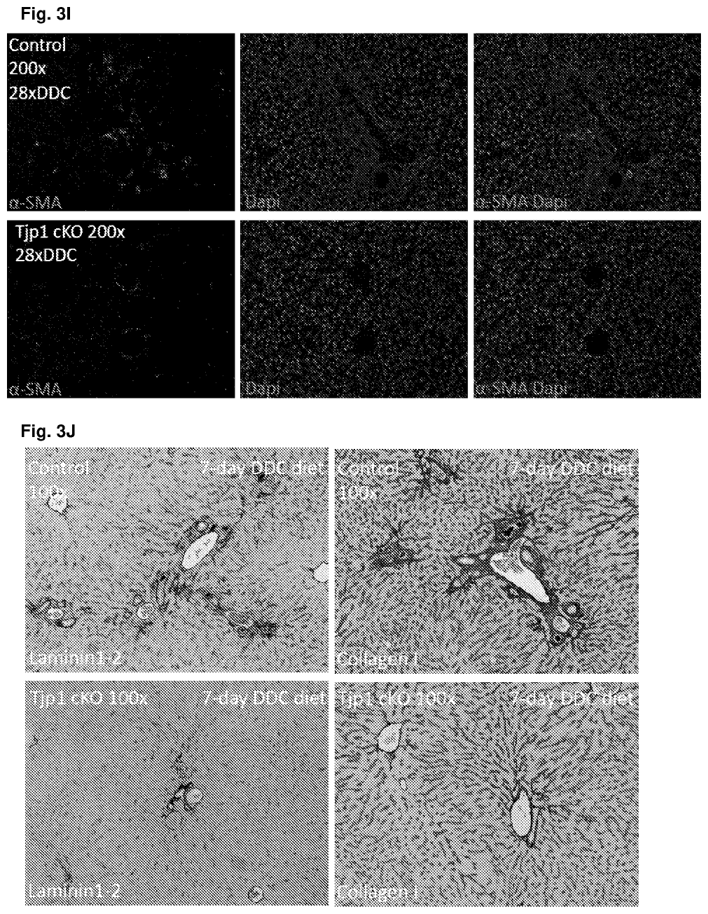

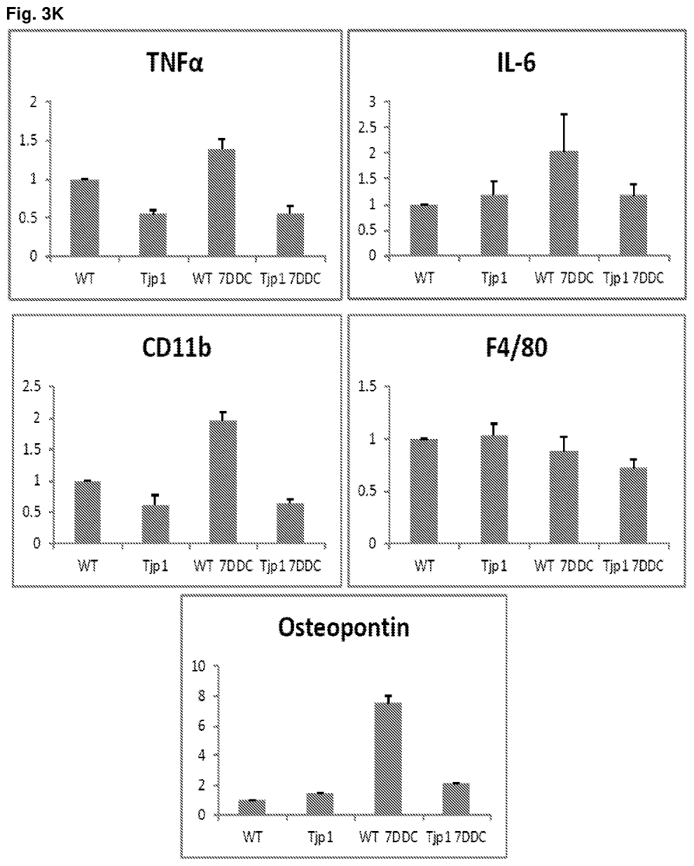

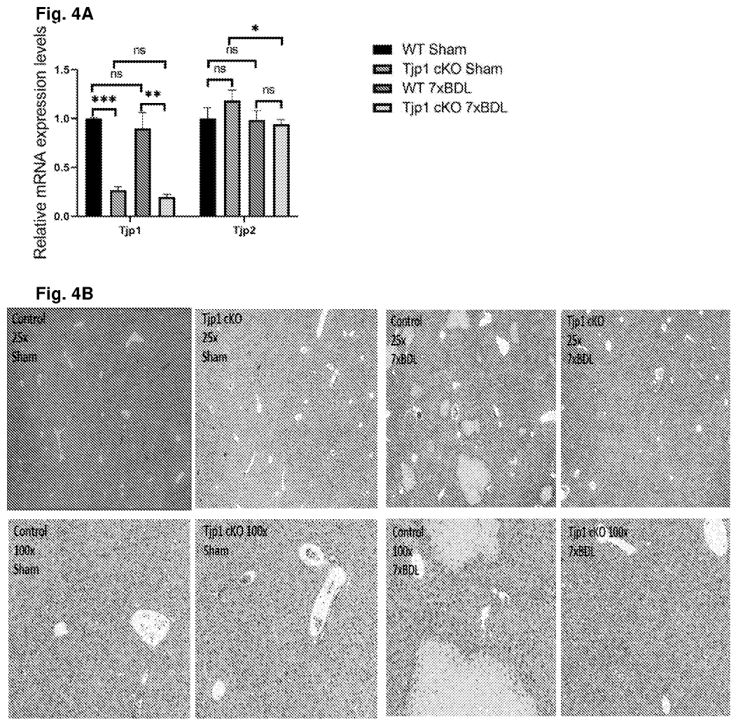

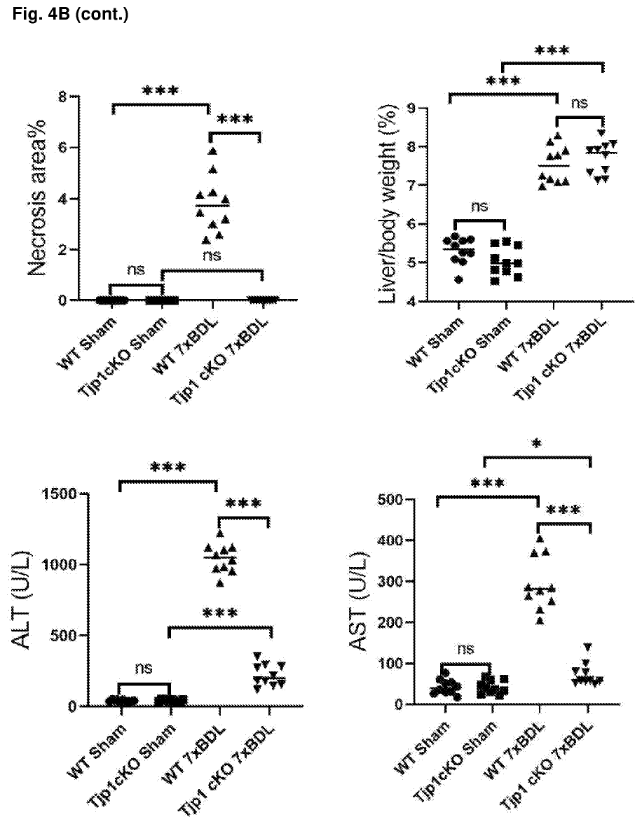

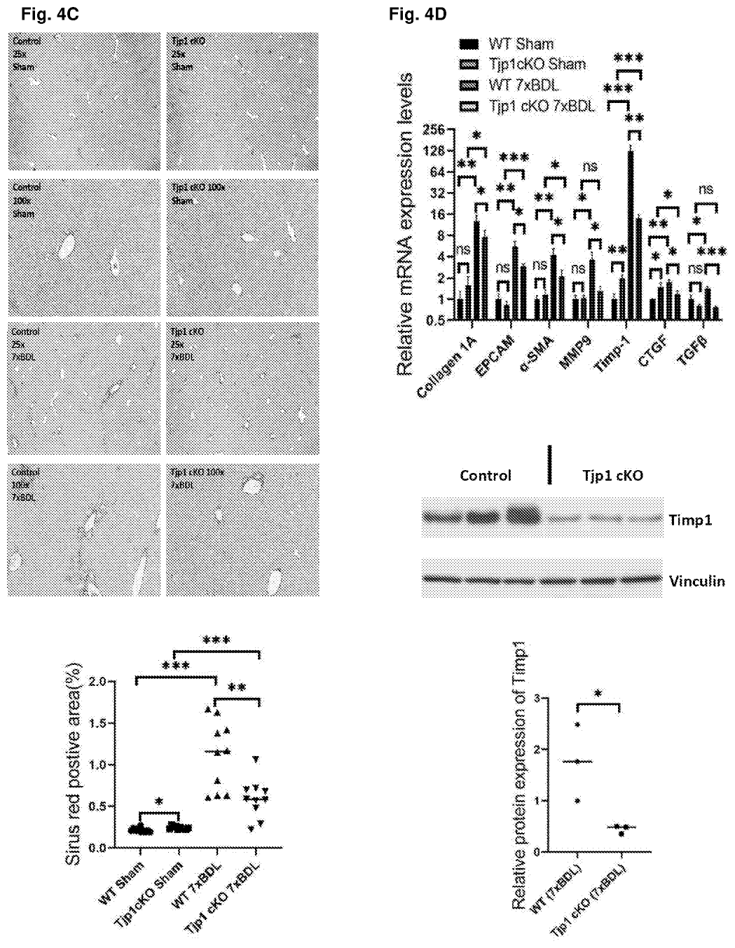

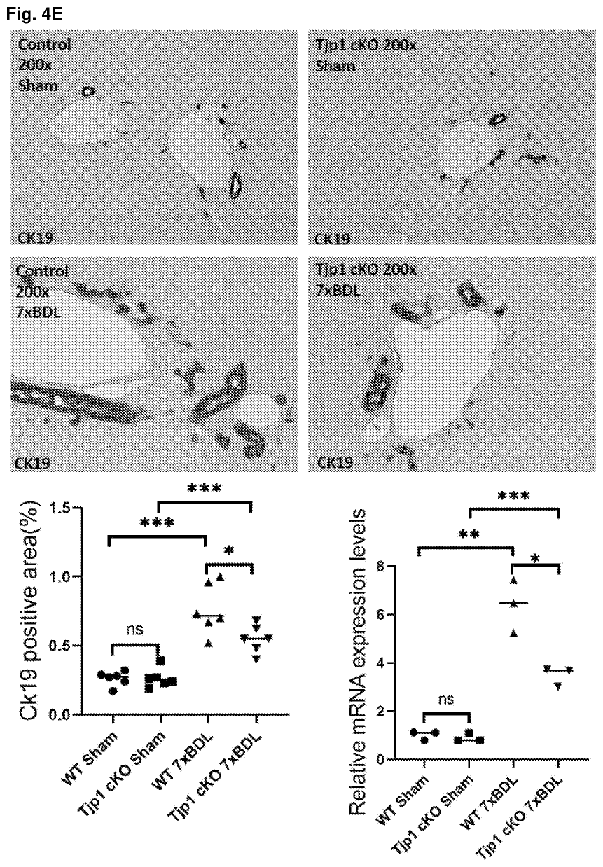

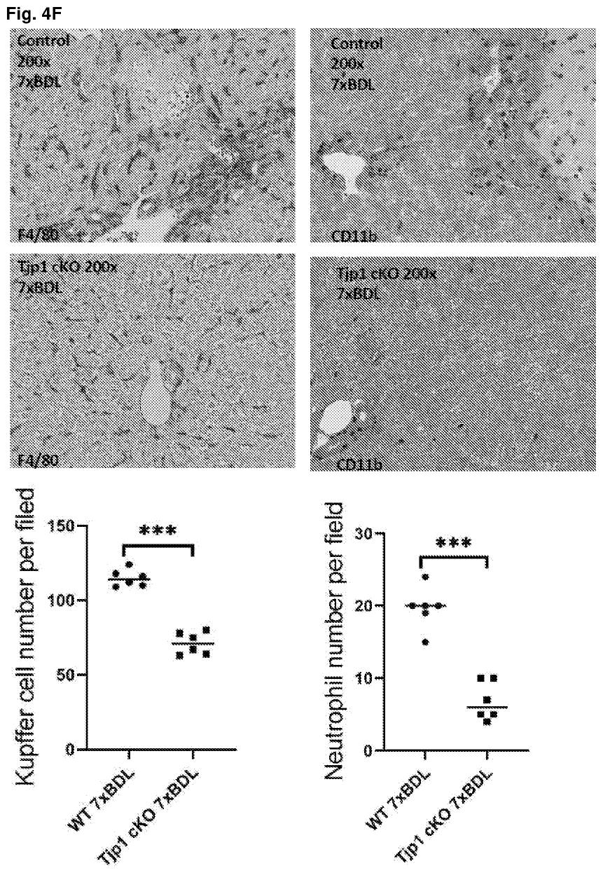

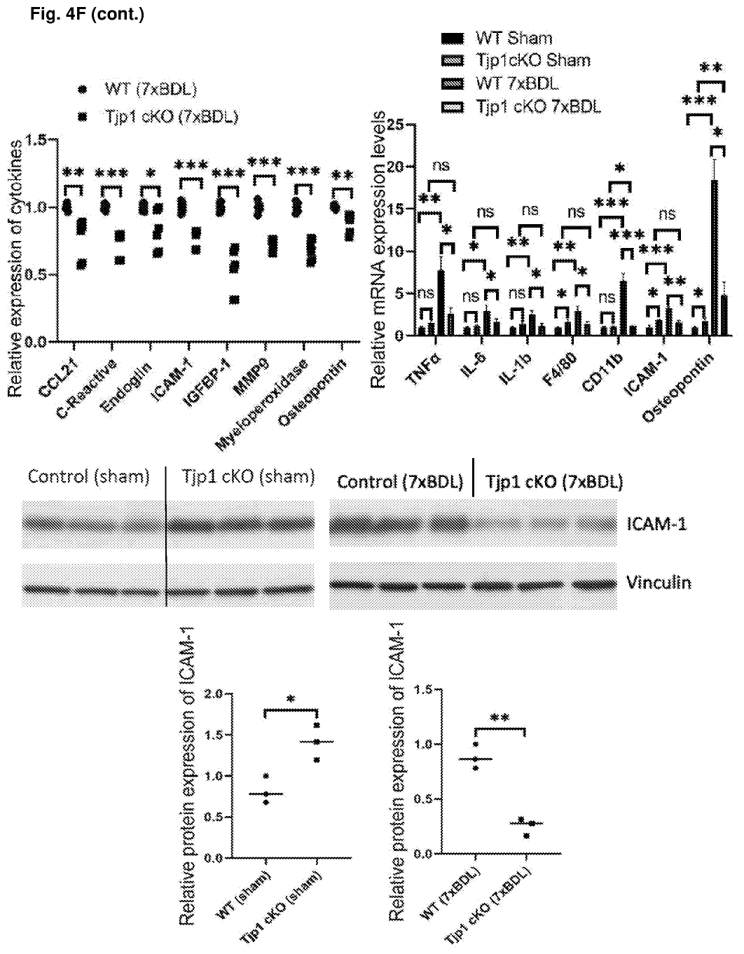

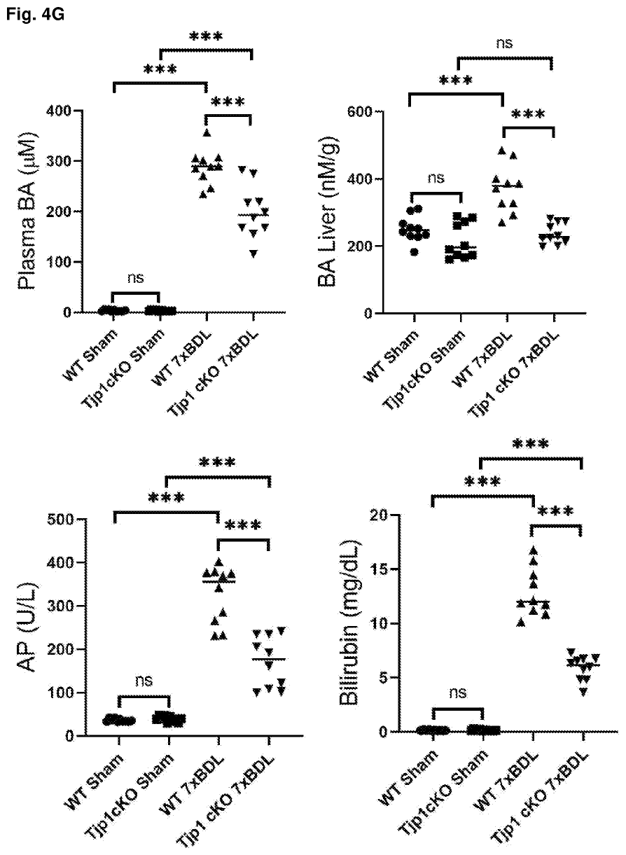

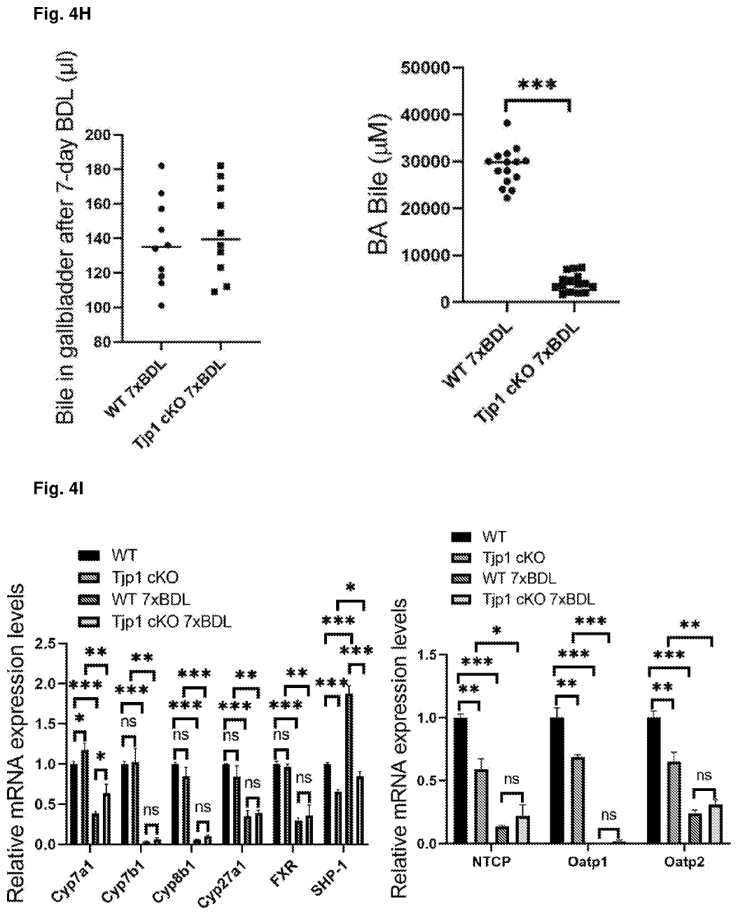

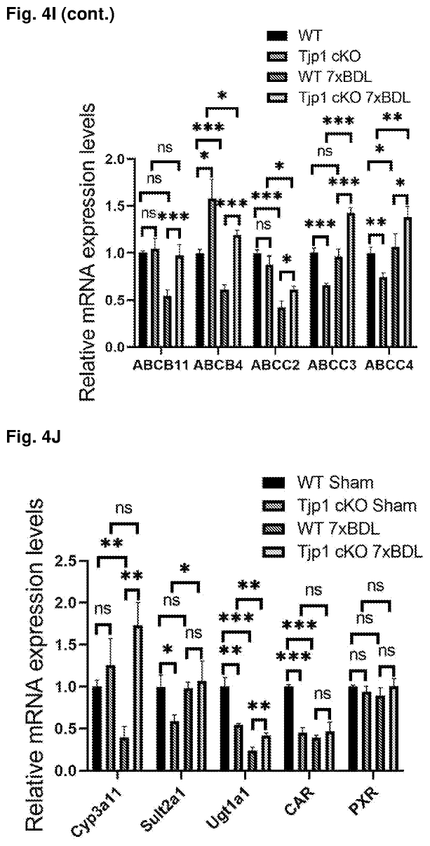

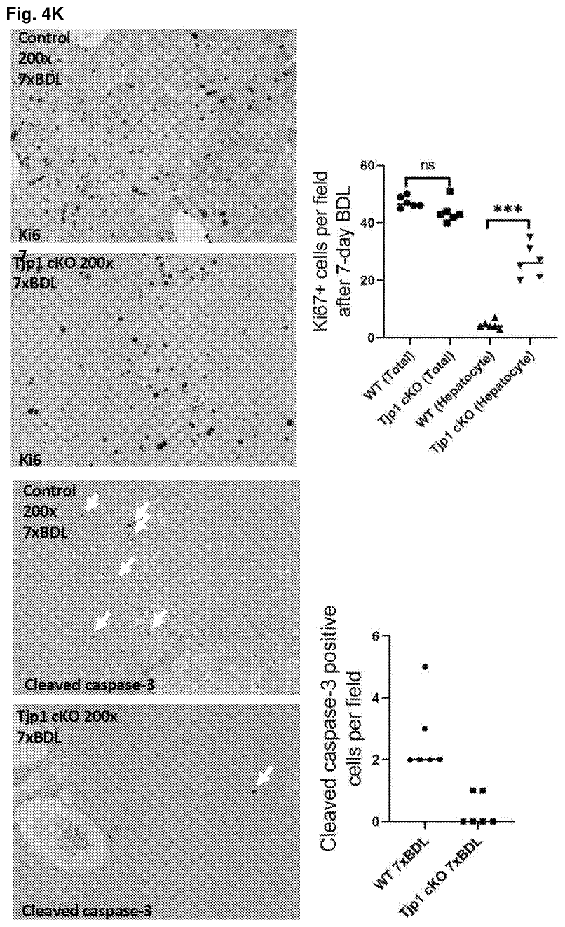

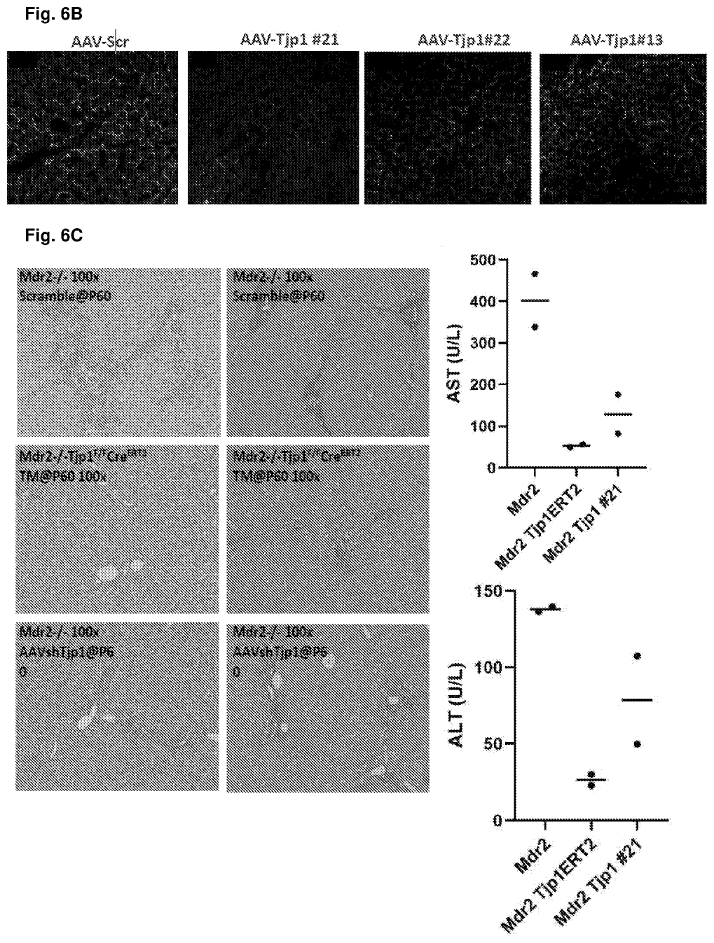

The detrimental effects of TAA (FIG. 2), DDC (FIG. 3) diet feeding, and bile duct ligation (FIG. 4) on the liver can be observed from the plasma and liver biochemistry, liver function, liver histopathology and liver fibrosis observed in the control wild type mice. These effects were abrogated in the Tjp1 cKO mice. Similarly, Yap cKO and Mdr2 KO mice also showed signs of diseased and damaged livers. The extent of damage was reduced after deleting Tjp1 in Yap cKO (FIG. 5) and Mdr2 KO (FIG. 6) mice.

In Yap cKO mice, the concomitant inactivation of Tjp1 resulted in the regeneration of a biliary system, shown by the positive CK19 staining which represents the proliferation of bile ducts (FIG. 5G). While CK19 can be used to show the presence of bile ducts, increased levels of CK19 staining when compared to a wild type control presents a different interpretation as it represents the proliferation of reactive bile ducts in biliary disorders or when induced by liver injuries, otherwise known as Ductular reaction (FIG. 3F).

In the DDC diet induced liver injury, BDL induced liver injury and Yap KO models, macrophage and neutrophil recruitment and inflammatory cytokine and chemokine levels were reduced in the ligated Tjp1 cKO liver (FIGS. 3K, 4F and 5F). Several bile acid synthesis (e.g. Cyp7a1) and transporter (e.g. Abcb11, Abcb4, Abcc2 and Abcc3) genes were upregulated in ligated Tjp1 cKO livers as assessed by qRT-PCR (FIGS. 41 and 4J). On the other hand, the expression of some of these transporter genes was lower in sham operated Tjp1 cKO livers in comparison to control livers, indicating a critical role of injury in their upregulation in the Tjp1 cKO livers. Similar changes in BA transporters and detoxification enzymes were also observed in other liver injury models after inactivation of Tjp1. In the BDL model, the concentrations of total and individual bile acids were reduced in the ligated Tjp1 cKO liver, suggesting lower hepatotoxicity when compared to ligated controls (Table 1 and FIG. 4H). These findings suggest that the deletion of Tjp1 confers protective effects on liver injury induced by hepatotoxic compounds, BDL or gene inactivation.

TABLE 1
Concentration of bile acids (nM) in BDL mice with and without
Tjp1 inactivation. n.s. stands for not significant.

| Bile acid name | Control (nM) | Tjp1 cKO (nM) | P value |
|---|---|---|---|
| α-muricholic acid | 17.35 ± 9.60 | 9.40 ± 1.48 | P < 0.05 |
| (α-MCA) | | | |
| β-muricholic acid | 171.48 ± 125.16 | 79.82 ± 42.40 | P < 0.05 |
| (β-MCA) | | | |
| ω-muricholic acid | 39.54 ± 23.44 | 14.51 ± 4.59 | P < 0.005 |
| (ω-MCA) | | | |
| Cholic acid (CA) | 9.76 ± 7.61 | 5.23 ± 0.39 | n.s. |
| Glycocheno- | 1.57 ± 0.40 | 1.31 ± 0.17 | n.s. |
| deoxycholic | | | |
| acid (GCDCA) | | | |
| Glycohyocholic | 23.81 ± 22.44 | 3.53 ± 0.72 | P < 0.05 |
| acid (GHCA) | | | |
| Glycocholic | 41.98 ± 35.49 | 13.35 ± 4.18 | P < 0.05 |
| acid (GCA) | | | |
| Taurolithocholic | 3.04 ± 1.66 | 1.23 ± 0.46 | P < 0.005 |
| acid (TLCA) | | | |
| Taurodeoxycholic | 47.31 ± 79.74 | 6.49 ± 4.58 | n.s. |
| acid (TDCA) | | | |
| Taurourso- | 1303.81 ± 856.82 | 245.75 ± 99.60 | P < 0.005 |
| deoxycholic | | | |
| acid (TUDCA) | | | |
| Tauro-b- | 291835 ± 222119 | 56785 ± 13824 | P < 0.005 |
| muricholic | | | |
| acid (T-b-MCA) | | | |
| Taurocholic | 130790 ± 117325 | 35640 ± 8086 | P < 0.05 |
| acid (TCA) | | | |

### Validation of Tjp1 as a Therapeutic Target for Cholestasis

In order to determine if the protective effect of knocking-out Tjp1 could be recapitulated by silencing Tjp1 specifically in the liver using AAV8 vectors, several DNAs encoding shTJP1 RNAs were screened in mouse hepatocyte cells. Three DNAs encoding shTjp1 RNAs that showed effective silencing of Tjp1 expression in tissue culture cells (FIG. 6A) were used to generate AAV8 vectors comprising the DNA encoding shTjp1 shRNA, in which expression of the shRNA is driven by a hepatocyte specific promotor (e.g. thyroxine binding globulin (TBG)) to ensure hepatocyte-specific silencing of Tjp1. Mice were then injected with different doses of AAV8 with DNAs encoding shTjp1 or scrambled shRNA at 2-months of age. AAV8 with DNA encoding shTjp1 #21 showed significant silencing of Tjp1 expression in the liver (FIG. 6B). When injected into Mdr2 KO mice, AAV8 with DNA encoding shTjp1 #21 was able to reduce serum AST and ALT levels, as well as liver fibrosis (FIG. 6C). Improvements to the efficacy can be made by identifying more efficient DNAs encoding Tjp1 shRNAs and/or the dosing of the AAV8 vector to enhance silencing or infection efficiency, respectively. Different chemistries of gene silencing or exon-skipping antisense oligonucleotides with a liver specific targeting module (e.g. GalNAc) can represent an alternative modality.

The siRNAs of SEQ ID NOs: 94-103 can, when injected into the bile duct ligated or the Yap cKO mouse models, inhibit Tjp1 and establish the beneficial effect on cholestasis (data not shown).

Inactivation of Tjp1 in the liver did not noticeably affect liver development, nor liver histology of function, as assessed by blood biochemistry and histological assays. At the cellular level, the expression and localization of key markers for tight junctions and cell polarity were normal. The structural and functional integrity of tight junctions, in particular the bile-blood barrier, also remained intact. Based on the results, the livers of the Tjp1 cKO mice did not exhibit any phenotype differences when compared to the livers of the wild type mice. The Tjp1 cKO mice however were conferred protection from adverse effects when subjected to different types of liver injury protocols. Tjp1 cKO mice showed better blood and liver biochemistry, less liver fibrosis and inflammation when compared to the corresponding control mice. This was attributed to the absence of Tjp1 from hepatocytes. In Yap cKO mice, concomitant inactivation of Tjp1 rescued the formation of a biliary system. In Mdr2 KO animals, hepatic deletion of Tjp1 suppressed liver injury and fibrosis. The beneficial effect of knocking out Tjp1 in the liver could be recapitulated using a therapeutic modality in the form of a DNA encoding Tjp1 shRNA expressed from a liver specific promotor and delivered in an AAV8 vector.

### Inactivation of Tjp1 Suppresses Hepatocellular Carcinoma (HCC)

As outlined above, inactivating Tjp1 in the liver is protective against different types of liver insults. Chronic exposure to such insults can lead to liver cancer. To assess whether Tjp1 would have a protective role in liver cancer, Tjp1 was inactivated in Mdr2/Abcb4 KO mice. Mdr2/Abcb4 KO is a well-established liver cirrhosis cancer model, where animals spontaneously develop hepatocellular carcinoma (HCC) by 12 months of age (P360). Sirius red staining of liver samples from mice at 6 months of age (P180) showed extensive fibrosis in the liver Mdr2 KO mice (FIG. 7). In contrast, little to no fibrosis was observed in the liver of Tjp1 Mdr2 cKO mice, which were indistinguishable from those of wild-type controls (FIG. 7). The livers from P360 mice and mice of up to 18 months of age were observed, and no tumors were detected in the Tjp1 cKO Mdr2 KO livers. This result contrasts with the livers from the control Mdr2 KO mice, which developed multiple hepatocellular tumors. Thus, in this HCC model, deletion of Tjp1 suppresses liver carcinogenesis, likely by attenuating liver fibrosis and inflammation.

As shown above, hepatic deletion of Tjp1 in Mdr2 KO mice suppressed the onset of hepatocellular cancer. Tjp1 is known to be considered as a tumor suppressor. It was previously shown that decreased Tjp1 expression was associated with tumor metastasis of liver cancer. However, the Tjp1/ZO-1 expression was analyzed in established tumors and not during the early stages of tumorigenesis. Therefore, the downregulation of Tjp1 expression in an established tumor may be linked to metastasis, rather than to tumor development itself. It was also shown that that ZO-1 is downregulated in liver cancer and only upregulated again in metastasis. This could be the result of carcinogenesis and has nothing to do with modulating the process of carcinogenesis. Merely showing the up- or down-regulation in cancer, or correlation with a process does not prove that a gene/protein is an oncogene or tumor suppressor, nor does it show that modulation of its expression affects tumorigenesis. The present application uses mice with healthy livers and the results show that inactivation of Tjp1 has a protective function against liver diseases, including liver carcinogenesis. It can therefore be appreciated that a therapeutic target against Tjp1 can be used to treat liver diseases and/or liver cancer.

Table 2 below details the SEQ ID Nos referenced herein and their corresponding sequences. A brief description of the sequences is also provided.

| SEQ | | |
|---|---|---|
| ID | Sequence | |
| NO. | (Nucleotide sequences are in the 5'->3' direction) | Description |
| 1 | CCGGCGTGGATTGAACTTACTAAATCTCGAGATTT | Short hairpin |
| | AGTAAGTTCAATCCACGTTTTTG | molecule encoding |
| | | TJP1 shRNA#18. |
| | | (Residues in bold |
| | | correspond to Tjp 1 |
| | | binding site 1; |
| | | residues in |
| | | underline |
| | | correspond to the |
| | | complementary |
| | | sequence of the |
| | | residues in bold) |
| 2 | CCGGCCGCGAAGTTATGAGCAAGTTCTCGAGAACT | Short hairpin |
| | TGCTCATAACTTCGCGGTTTTTG | molecule encoding |
| | | TJP1 shRNA#21. |
| | | (Residues in bold |
| | | correspond to Tjp 1 |
| | | binding site 2; |
| | | residues in |
| | | underline |
| | | correspond to the |
| | | complementary |
| | | sequence of the |
| | | residues in bold) |
| 3 | CCGGCGGCCATTTGAACGCAAATTTCTCGAGAAAT | Short hairpin |
| | TTGCGTTCAAATGGCCGTTTTTG | molecule encoding |
| | | TJP1 shRNA #22. |
| | | (Residues in bold |
| | | correspond to Tjp1 |
| | | binding site 3; |
| | | residues in |
| | | underline |
| | | correspond to the |
| | | complementary |
| | | sequence of the |
| | | residues in bold) |
| 4 | CGTGGATTGAACTTACTAAAT | Tjp1 binding site 1 |
| | | -corresponds to |
| | | residues of 5678- |
| | | 5698 of SEQ ID |
| | | NO: 10 |
| 5 | CCGCGAAGTTATGAGCAAGTT | Tjp1 binding site 2 |
| | | -corresponds to |
| | | residues 3986- |
| | | 4006 of SEQ ID |
| | | NO: 10 |
| 6 | CGGCCATTTGAACGCAAATTT | Tjp1 binding site 3 |
| | | -corresponds to |
| | | residues 5003- |
| | | 5023 of SEQ ID |
| | | NO: 10 |
| 7 | gccgccgggc cacggcctgc aggggtggaa ggggcggcgg cggcgggagc | Human Tjp 1 DNA |
| | tgcgggccac gcacggcggc ggcggcggga gcggccgggc acgcacggcg | sequence. |
| | acggcggcgg cgggagcggg agcccgggcc acgcacggcc gcagctgcgg | (Residues in |
| | gtgcggcggg cgcgggagcg gtgggagctg tggtggcggc ggatggccca | italics |
| | gagctgatct atgcggcgcc tggaggggct gtgtctgcgg cggcggcgga | correspond to the |
| | ggcggcgggg ccctgcccgc gagcggagcg gggacaagat gaagtaccag | open reading |
| | aaatacctga cggtgctgca gatggccatc ggcgtcaccc cctccaaccg | frame.) |
| | cggcagcctc ctgccgctca agaggaagct gtgggtaacg ccatcctctg | |
| | aaaatcctaa tggtgctact tctagtgtca gccaaggaaa accctcttta | |
| | agacgaatta aagggagatt acacagaagc aaaagccttg atagcatgga | |
| | tttctgtgag ctcactagca cagcaatgga ggaaacagct atatgggaac | |
| | aacatacagt gacgcttcac agggctcctg gatttggatt tggaattgca | |
| | atatctggtg gacgagataa tcctcatttt cagagtgggg aaacgtcaat | |
| | agtgatttca gatgtgctga aaggaggacc agctgaagga cagctacagg | |
| | aaaatgaccg agttgcaatg gttaacggag tttcaatgga taatgttgaa | |
| | catgcttttg ctgttcagca actaaggaaa agtgggaaaa atgcaaaaat | |
| | tacaattaga aggaagaaga aagttcaaat accagtaagt cgtcctgatc | |
| | ctgaaccagt atctgataat gaagaagata gttatgatga ggaaatacat | |
| | gatccaagaa gtggccggag tggtgtggtt aacagaagga gtgagaagat | |
| | ttggccgagg gatagaagtg caagtagaga gaggagcttg tccccgcggt | |
| | cagacaggcg gtcagtggct tccagccagc ctgctaaacc tactaaagtc | |
| | acactggtga aatcccggaa aaatgaagaa tatggtcttc gattggcaag | |
| | ccatatattt gttaaggaaa tttcacaaga tagtttggca gcaagagatg | |
| | gcaatattca agaaggtgat gttgtattga agataaatgg tactgtgaca | |
| | gaaaatatgt cattgacaga tgcaaagaca ttgatagaaa ggtctaaagg | |
| | caaattaaaa atggtagttc aaagagatga acgggctacg ctattgaatg | |
| | tccctgatct ttctgacagc atccactctg ctaatgcctc tgagagagac | |
| | gacatttcag aaattcagtc actggcatca gatcattctg gtcgatcaca | |
| | cgataggcct ccccgccgca gccggtcacg atctcctgac cagcggtcag | |
| | agccttctga tcattccagg cactcgccgc agcagccaag caatggcagt | |
| | ctccggagta gagatgaaga gagaatttct aaacctgggg ctgtctcaac | |
| | tcctgtaaag catgctgatg atcacacacc taaaacagtg gaagaagtta | |
| | cagttgaaag aaatgagaaa caaacacctt ctcttccaga accaaagcct | |
| | gtgtatgccc aagttgggca accagatgtg gatttacctg tcagtccatc | |
| | tgatggtgtc ctacctaatt caactcatga agatgggatt cttcggccca | |
| | gcatgaaatt ggtaaaattc agaaaaggag atagtgtggg tttgcggctg | |
| | gctggtggaa atgatgttgg aatatttgta gctggcgttc tagaagatag | |
| | ccctgcagcc aaggaaggct tagaggaagg tgatcaaatt ctcagggtaa | |
| | acaacgtaga ttttacaaat atcataagag aagaagccgt ccttttcctg | |
| | cttgacctcc ctaaaggaga agaagtgacc atattggctc agaagaagaa | |
| | ggatgtttat cgtcgcattg tagaatcaga tgtaggagat tctttctata ttagaaccca | |
| | ttttgaatat gaaaaggaat ctccctatgg acttagtttt aacaaaggag | |
| | aggtgttccg tgttgtggat accttgtaca atggaaaact gggctcttgg cttgctattc | |
| | gaattggtaa aaatcataag gaggtagaac gaggcatcat ccctaataag | |
| | aacagagctg agcagctagc cagtgtacag tatacacttc caaaaacagc | |
| | aggcggagac cgtgctgact tctggagatt cagaggtctt cgcagctcca | |
| | agagaaatct tcgaaaaagc agagaggatt tgtccgctca gcctgttcaa | |
| | acaaagtttc cagcttatga aagagtggtt cttcgagaag ctggatttct | |
| | gaggcctgta accatttttg gaccaatagc tgatgttgcc agagaaaagc | |
| | tggcaagaga agaaccagat atttatcaaa ttgcaaagag tgaaccacga | |
| | gacgctggaa ctgaccaacg tagctctggc attattcgcc tgcatacaat | |
| | aaagcaaatc atagatcaag acaaacatgc tttattagat gtaacaccaa | |
| | atgcagttga tcgtcttaac tatgcccagt ggtatccaat tgttgtattt cttaaccctg | |
| | attctaagca aggagtaaaa acaatgagaa tgaggttatg tccagaatct | |
| | cggaaaagtg ccaggaagtt atacgagcga tctcataaac ttcgtaaaaa | |
| | taatcaccat ctttttacaa ctacaattaa cttaaattca atgaatgatg gttggtatgg | |
| | tgcgctgaaa gaagcaattc aacaacagca aaaccagctg gtatgggttt | |
| | ccgagggaaa ggcggatggt gctacaagtg atgaccttga tttgcatgat | |
| | gatcgtctgt cctacctgtc agctccaggt agtgaatact caatgtatag | |
| | cacggacagt agacacactt ctgactatga agacacagac acagaaggcg | |
| | gggcctacac tgatcaagaa ctagatgaaa ctcttaatga tgaggttggg | |
| | actccaccgg agtctgccat tacacggtcc tctgagcctg taagagagga | |
| | ctcctctgga atgcatcatg aaaaccaaac atatcctcct tactcaccac | |
| | aagcgcagcc acaaccaatt catagaatag actcccctgg atttaagcca | |
| | gcctctcaac agaaagcaga agcttcatct ccagtccctt acctttcgcc | |
| | tgaaacaaac ccagcatcat caacctctgc tgttaatcat aatgtaaatt | |
| | taactaatgt cagactggag gagcccaccc cagctccttc cacctcttac | |
| | tcaccacaag ctgattcttt aagaacacca agtactgagg cagctcacat | |
| | aatgctaaga gatcaagaac catcattgtc gtcgcatgta gatccaacaa | |
| | aggtgtatag aaaggatcca tatcccgagg aaatgatgag gcagaaccat | |
| | gttttgaaac agccagccgt tagtcaccca gggcacaggc cagacaaaga | |
| | gcctaatctg acctatgaac cccaactccc atacgtagag aaacaagcca | |
| | gcagagacct cgagcagccc acatacagat acgagtcctc aagctatacg | |
| | gaccagtttt ctcgaaacta tgaacatcgt ctgcgatacg aagatcgcgt | |
| | ccccatgtat gaagaacagt ggtcatatta tgatgacaaa cagccctacc | |
| | catctcggcc accttttgat aatcagcact ctcaagacct tgactccaga | |
| | cagcatcccg aagagtcctc agaacgaggg tactttccac gttttgaaga | |
| | gccagcccct ctgtcttacg acagcagacc acgttacgaa caggcaccta | |
| | gagcatccgc cctgcggcac gaagagcagc cagctcctgg gtatgacaca | |
| | catggtagac tcagaccgga agcccagccc cacccttcag cagggcccaa | |
| | gcctgcagag tccaagcagt attttgagca atattcacgc agttacgagc | |
| | aagtaccacc ccaaggattt acctctagag caggtcattt tgagcctctc | |
| | catggtgctg cagctgtccc tccgctgata ccttcatctc agcataagcc | |
| | agaagctctg ccttcaaaca ccaaaccact gcctccaccc ccaactcaaa | |
| | ccgaagaaga ggaagatcca gcaatgaagc cacagtctgt actcaccaga | |
| | gttaagatgt ttgaaaacaa aagatctgca tccttagaga ccaagaagga | |
| | tgtaaatgac actggcagtt ttaagcctcc agaagtagca tctaaacctt | |
| | caggtgctcc catcattggt cccaaaccca cttctcagaa tcaattcagt | |
| | gaacatgaca aaactctgta caggatccca gaacctcaaa aacctcaact | |
| | gaagccacct gaagatattg ttcggtccaa tcattatgac cctgaagaag | |
| | atgaagaata ttatcgaaaa cagctgtcat actttgaccg aagaagtttt | |
| | gagaataagc ctcctgcaca cattgccgcc agccatctct ccgagcctgc | |
| | aaagccagcg cattctcaga atcaatcaaa tttttctagt tattcttcaa | |
| | agggaaagcc tcctgaagct gatggtgtgg atagatcatt tggcgagaaa | |
| | cgctatgaac ccatccaggc cactccccct cctcctccat tgccctcgca | |
| | gtatgcccag ccatctcagc ctgtcaccag cgcgtctctc cacatacatt | |
| | ctaagggagc acatggtgaa ggtaattcag tgtcattgga ttttcagaat | |
| | tccttagtgt ccaaaccaga cccacctcca tctcagaata agccagcaac | |
| | tttcagacca ccaaaccgag aagatactgc tcaggcagct ttctatcccc | |
| | agaaaagttt tccagataaa gccccagtta atggaactga acagactcag | |
| | aaaacagtca ctccagcata caatcgattc acaccaaaac catatacaag | |
| | ttctgcccga ccatttgaac gcaagtttga aagtcctaaa ttcaatcaca | |
| | atcttctgcc aagtgaaact gcacataaac ctgacttgtc ttcaaaaact | |
| | cccacttctc caaaaactct tgtgaaatcg cacagtttgg cacagcctcc | |
| | tgagtttgac agtggagttg aaactttctc tatccatgca gagaagccta | |
| | aatatcaaat aaataatatc agcacagtgc ctaaagctat tcctgtgagt | |
| | ccttcagctg tggaagagga tgaagatgaa gatggtcata ctgtggtggc | |
| | cacagcccga ggcatattta acagcaatgg gggcgtgctg agttccatag | |
| | aaactggtgt tagtataatt atccctcaag gagccattcc cgaaggagtt | |
| | gagcaggaaa tctatttcaa ggtctgccgg gacaacagca tccttccacc | |
| | tttagataaa gagaaaggtg aaacactgct gagtcctttg gtgatgtgtg | |
| | gtccccatgg cctcaagttc ctgaagcctg tggagctgcg cttaccacac | |
| | tgtgcgtcca tgactcctga cggttggtct tttgctctaa aatcatccga ctcctcgtcg | |
| | ggtgatccta aaacctggca aaacaagtgt cttcccggag atccaaatta | |
| | tctcgttgga gcaaactgtg tttctgtcct tattgaccac ttttaactct tgaaatatag | |
| | gaacttaaat aatgtgaaac tggattaaac ttaatctaaa tggaaccact | |
| | ctatcaagta ttataccttt tttagagttg atactacagt ttgttagtat gaggcatttg | |
| | tttgaactga taaagatgag tgagcatgcc cctgaaccat ggtcggaaaa | |
| | catgctacac actgcatgtt tgtgattgac gggactgttg gtattggcta | |
| | gaggttcaaa gatattttgc tttgtgattt ttgtaatttt tttatcgtca ctgcttaact | |
| | tcacatattg atttccgtta aaataccagc cagtaaatgg gggtgcattt gaggtctgtt | |
| | ctttccaaag tacactgttt caaactttac tatggccctg gcctagcata cgtacacatt | |
| | ttattttatt atgcatgaag taatatgcac acatttttta aatgcacctg gaatatataa | |
| | ccagtgttgt ggatttaaca gaaatgtaca gcaaggagat ttacaactgg | |
| | gggagggtga agtgaagaca atgacttact gtacatgaaa acacattttt | |
| | cttagggaag gatacaaaag catgtgagac tggttccatg gcctcttcag | |
| | atctctaact tcaccatatt accacagaca tactaaccag cagaaatgcc | |
| | ttaccctcat gttcttaatt cttagctcat tctccttgtg ttactaagtt tttatggctt | |
| | ttgtgcatta tctagatact gtatcatgac aaagactgag tacgttgtgc atttggtggt | |
| | ttcagaaatg tgttatcacc cagaagaaaa tagtggtgtg atttggggat atttttttct | |
| | tttcttttct tttctttttt tttttttttg acaaggggca gtggtggttt tctgttcttt ctggctatgc | |
| | atttgaaaat tttgatgttt taaggatgct tgtacataat gcgtgcatac cacttttgtt | |
| | cttggtttgt aaattaactt ttataaactt tacctttttt atacataaac aagaccacgt | |
| | ttctaaaggc tacctttgta ttctctcctg tacctcttga gccttgaact ttgacctctg | |
| | cagcaataaa gcagcgtttc tatgacacat gcaaggtcat tttttttaag | |
| | aaaaaggatg cacagagttg ttacattttt aagtgctgca tttaaaagat | |
| | acagttactc agaattctct agtttgatta aattcttgca aagtatccct actgtaattt | |
| | gtgatacaat gctgtgccct aaagtgtatt tttttactaa tagacaattt attatggcac | |
| | atcagcacga tttctgttta gataatacac cactacattc tgttaatcat taggtgtgac | |
| | tgaatttctt ttgccgttat taaaaatctc aaatttctaa atctccaaaa taaaactttt | |
| | taaaataaag tgctggcttg gtctgtttgc ccactgtttt ctagtttcat gcagctttat | |
| | aatcctgttt taaaatcctg cacacaaatc cctatcaccc agcgtcacct | |
| | accacctcgt cgtctggtgt tgcatgcaga atttctcccc ttggccagca | |
| | tgtacagatg ggtgggcagt gctcatctga agggctcaga ctgaagtggg | |
| | gcagaaggac ctggagacag agtgggagaa ggcagcaggc cgacttcccc | |
| | ctgtgggtaa acacacaccc ctgcgtggag aaacacccct gcatggggac | |
| | acacgtgcgt ttgtgtgtgt gcgtgtaacc atttgtatat ggttttattc cccagataaa | |
| | tagcacgggc attgtttaat gtcacccaca tttgggggaa gaaaatgggt | |
| | ttagtgagca taaatattcc attgtggaca tcctacttac ttaactgcca cccctgttga | |
| | catttggatt attgctatag tgtttcagta aacacctttg tggactgaat ccatctatgt | |
| | ggattttatt tttttcatct ttgttaaggt ataatttaca tagagtaagt tactgttttt | |
| | aaaatgtata gttcaaattt tgacaaacac atactgttta tttaccacgg ttaatacata | |
| | caacagtttc tttatcttct ctaatcattt gtaccccttt atactcaacc cttcttccag | |
| | cccctggcag gatgatctct tctgtttcta tagttgcctt tttgacagtg tcatataaat | |
| | ggaatcatac aata | |
| 8 | atgcggcgcc tggaggggct gtgtctgcgg cggcggcgga ggcggcgggg | Human Tjp1 DNA |
| | ccctgcccgc gagcggagcg gggacaagat gaagtaccag aaatacctga | sequence-open |
| | cggtgctgca gatggccatc ggcgtcaccc cctccaaccg cggcagcctc | reading frame |
| | ctgccgctca agaggaagct gtgggtaacg ccatcctctg aaaatcctaa | (residues 211-5796 |
| | tggtgctact tctagtgtca gccaaggaaa accctcttta agacgaatta | of SEQ ID NO: 7) |
| | aagggagatt acacagaagc aaaagccttg atagcatgga tttctgtgag | |
| | ctcactagca cagcaatgga ggaaacagct atatgggaac aacatacagt | |
| | gacgcttcac agggctcctg gatttggatt tggaattgca atatctggtg | |
| | gacgagataa tcctcatttt cagagtgggg aaacgtcaat agtgatttca | |
| | gatgtgctga aaggaggacc agctgaagga cagctacagg aaaatgaccg | |
| | agttgcaatg gttaacggag tttcaatgga taatgttgaa catgcttttg ctgttcagca | |
| | actaaggaaa agtgggaaaa atgcaaaaat tacaattaga aggaagaaga | |
| | aagttcaaat accagtaagt cgtcctgatc ctgaaccagt atctgataat | |
| | gaagaagata gttatgatga ggaaatacat gatccaagaa gtggccggag | |
| | tggtgtggtt aacagaagga gtgagaagat ttggccgagg gatagaagtg | |
| | caagtagaga gaggagcttg tccccgcggt cagacaggcg gtcagtggct | |
| | tccagccagc ctgctaaacc tactaaagtc acactggtga aatcccggaa | |
| | aaatgaagaa tatggtcttc gattggcaag ccatatattt gttaaggaaa | |
| | tttcacaaga tagtttggca gcaagagatg gcaatattca agaaggtgat | |
| | gttgtattga agataaatgg tactgtgaca gaaaatatgt cattgacaga | |
| | tgcaaagaca ttgatagaaa ggtctaaagg caaattaaaa atggtagttc | |
| | aaagagatga acgggctacg ctattgaatg tccctgatct ttctgacagc | |
| | atccactctg ctaatgcctc tgagagagac gacatttcag aaattcagtc | |
| | actggcatca gatcattctg gtcgatcaca cgataggcct ccccgccgca | |
| | gccggtcacg atctcctgac cagcggtcag agccttctga tcattccagg | |
| | cactcgccgc agcagccaag caatggcagt ctccggagta gagatgaaga | |
| | gagaatttct aaacctgggg ctgtctcaac tcctgtaaag catgctgatg | |
| | atcacacacc taaaacagtg gaagaagtta cagttgaaag aaatgagaaa | |
| | caaacacctt ctcttccaga accaaagcct gtgtatgccc aagttgggca | |
| | accagatgtg gatttacctg tcagtccatc tgatggtgtc ctacctaatt caactcatga | |
| | agatgggatt cttcggccca gcatgaaatt ggtaaaattc agaaaaggag | |
| | atagtgtggg tttgcggctg gctggtggaa atgatgttgg aatatttgta gctggcgttc | |
| | tagaagatag ccctgcagcc aaggaaggct tagaggaagg tgatcaaatt | |
| | ctcagggtaa acaacgtaga ttttacaaat atcataagag aagaagccgt | |
| | ccttttcctg cttgacctcc ctaaaggaga agaagtgacc atattggctc | |
| | agaagaagaa ggatgtttat cgtcgcattg tagaatcaga tgtaggagat | |
| | tctttctata ttagaaccca ttttgaatat gaaaaggaat ctccctatgg acttagtttt | |
| | aacaaaggag aggtgttccg tgttgtggat accttgtaca atggaaaact | |
| | gggctcttgg cttgctattc gaattggtaa aaatcataag gaggtagaac | |
| | gaggcatcat ccctaataag aacagagctg agcagctagc cagtgtacag | |
| | tatacacttc caaaaacagc aggcggagac cgtgctgact tctggagatt | |
| | cagaggtctt cgcagctcca agagaaatct tcgaaaaagc agagaggatt | |
| | tgtccgctca gcctgttcaa acaaagtttc cagcttatga aagagtggtt | |
| | cttcgagaag ctggatttct gaggcctgta accattittg gaccaatagc | |
| | tgatgttgcc agagaaaagc tggcaagaga agaaccagat atttatcaaa | |
| | ttgcaaagag tgaaccacga gacgctggaa ctgaccaacg tagctctggc | |
| | attattcgcc tgcatacaat aaagcaaatc atagatcaag acaaacatgc | |
| | tttattagat gtaacaccaa atgcagttga tcgtcttaac tatgcccagt ggtatccaat | |
| | tgttgtattt cttaaccctg attctaagca aggagtaaaa acaatgagaa | |
| | tgaggttatg tccagaatct cggaaaagtg ccaggaagtt atacgagcga | |
| | tctcataaac ttcgtaaaaa taatcaccat ctttttacaa ctacaattaa cttaaattca | |
| | atgaatgatg gttggtatgg tgcgctgaaa gaagcaattc aacaacagca | |
| | aaaccagctg gtatgggttt ccgagggaaa ggcggatggt gctacaagtg | |
| | atgaccttga tttgcatgat gatcgtctgt cctacctgtc agctccaggt agtgaatact | |
| | caatgtatag cacggacagt agacacactt ctgactatga agacacagac | |
| | acagaaggcg gggcctacac tgatcaagaa ctagatgaaa ctcttaatga | |
| | tgaggttggg actccaccgg agtctgccat tacacggtcc tctgagcctg | |
| | taagagagga ctcctctgga atgcatcatg aaaaccaaac atatcctcct | |
| | tactcaccac aagcgcagcc acaaccaatt catagaatag actcccctgg | |
| | atttaagcca gcctctcaac agaaagcaga agcttcatct ccagtccctt | |
| | acctttcgcc tgaaacaaac ccagcatcat caacctctgc tgttaatcat | |
| | aatgtaaatt taactaatgt cagactggag gagcccaccc cagctccttc | |
| | cacctcttac tcaccacaag ctgattcttt aagaacacca agtactgagg | |
| | cagctcacat aatgctaaga gatcaagaac catcattgtc gtcgcatgta | |
| | gatccaacaa aggtgtatag aaaggatcca tatcccgagg aaatgatgag | |
| | gcagaaccat gttttgaaac agccagccgt tagtcaccca gggcacaggc | |
| | cagacaaaga gcctaatctg acctatgaac cccaactccc atacgtagag | |
| | aaacaagcca gcagagacct cgagcagccc acatacagat acgagtcctc | |
| | aagctatacg gaccagtttt ctcgaaacta tgaacatcgt ctgcgatacg | |
| | aagatcgcgt ccccatgtat gaagaacagt ggtcatatta tgatgacaaa | |
| | cagccctacc catctcggcc accttttgat aatcagcact ctcaagacct | |
| | tgactccaga cagcatcccg aagagtcctc agaacgaggg tactttccac | |
| | gttttgaaga gccagcccct ctgtcttacg acagcagacc acgttacgaa | |
| | caggcaccta gagcatccgc cctgcggcac | |
| | gaagagcagc cagctcctgg gtatgacaca catggtagac tcagaccgga | |
| | agcccagccc cacccttcag cagggcccaa gcctgcagag tccaagcagt | |
| | attttgagca atattcacgc agttacgagc aagtaccacc ccaaggattt | |
| | acctctagag caggtcattt tgagcctctc catggtgctg cagctgtccc | |
| | tccgctgata ccttcatctc agcataagcc agaagctctg ccttcaaaca | |
| | ccaaaccact gcctccaccc ccaactcaaa ccgaagaaga ggaagatcca | |
| | gcaatgaagc cacagtctgt actcaccaga gttaagatgt ttgaaaacaa | |
| | aagatctgca tccttagaga ccaagaagga tgtaaatgac actggcagtt | |
| | ttaagcctcc agaagtagca tctaaacctt caggtgctcc catcattggt | |
| | cccaaaccca cttctcagaa tcaattcagt gaacatgaca aaactctgta | |
| | caggatccca gaacctcaaa aacctcaact gaagccacct gaagatattg | |
| | ttcggtccaa tcattatgac cctgaagaag atgaagaata ttatcgaaaa | |
| | cagctgtcat actttgaccg aagaagtttt gagaataagc ctcctgcaca | |
| | cattgccgcc agccatctct ccgagcctgc aaagccagcg cattctcaga | |
| | atcaatcaaa tttttctagt tattcttcaa agggaaagcc tcctgaagct gatggtgtgg | |
| | atagatcatt tggcgagaaa cgctatgaac ccatccaggc cactccccct | |
| | cctcctccat tgccctcgca gtatgcccag ccatctcagc ctgtcaccag | |
| | cgcgtctctc cacatacatt ctaagggagc acatggtgaa ggtaattcag | |
| | tgtcattgga ttttcagaat tccttagtgt ccaaaccaga cccacctcca | |
| | tctcagaata agccagcaac tttcagacca ccaaaccgag aagatactgc | |
| | tcaggcagct ttctatcccc agaaaagttt tccagataaa gccccagtta | |
| | atggaactga acagactcag aaaacagtca ctccagcata caatcgattc | |
| | acaccaaaac catatacaag ttctgcccga ccatttgaac gcaagtttga | |
| | aagtcctaaa ttcaatcaca atcttctgcc aagtgaaact gcacataaac | |
| | ctgacttgtc ttcaaaaact cccacttctc caaaaactct tgtgaaatcg | |
| | cacagtttgg cacagcctcc tgagtttgac agtggagttg aaactttctc | |
| | tatccatgca gagaagccta aatatcaaat aaataatatc agcacagtgc | |
| | ctaaagctat tcctgtgagt ccttcagctg tggaagagga tgaagatgaa | |
| | gatggtcata ctgtggtggc cacagcccga ggcatattta acagcaatgg | |
| | gggcgtgctg agttccatag aaactggtgt tagtataatt | |
| | atccctcaag gagccattcc cgaaggagtt gagcaggaaa tctatttcaa | |
| | ggtctgccgg gacaacagca tccttccacc tttagataaa gagaaaggtg | |
| | aaacactgct gagtcctttg gtgatgtgtg gtccccatgg cctcaagttc | |
| | ctgaagcctg tggagctgcg cttaccacac tgtgcgtcca tgactcctga | |
| | cggttggtct tttgctctaa aatcatccga ctcctcgtcg ggtgatccta | |
| | aaacctggca aaacaagtgt cttcccggag atccaaatta tctcgttgga | |
| | gcaaactgtg tttctgtcct tattgaccac ttttaa | |
| 9 | MRRLEGLCLRRRRRRRGPARERSGDKMKYQKYLTVLQMAIG | Human Tjp1 amino |
| | VTPSTAMEETAIWEQHTVTLHRAPGFGFGIAISGGRDNPHFQS | acid sequence |
| | GETSIVISDVLKGGPAEGQLQENDRVAMVNGVSMDNVEHAFA | |
| | VQQLRKSGKNAKITIRRKKKVQIPVSRPDPEPVSDNEEDSYDE | |
| | EIHDPRSGRSGVVNRRSEKIWPRDRSASRERSLSPRSDRRSV | |
| | ASSQPAKPTKVTLVKSRKNEEYGLRLASHIFVKEISQDSLAARD | |
| | GNIQEGDVVLKINGTVTENMSLTDAKTLIERSKGKLKMVVQRD | |
| | ERATLLNVPDLSDSIHSANASERDDISEIQSLASDHSGRSHDR | |
| | PPRRSRSRSPDQRSEPSDHSRHSPQQPSNGSLRSRDEERIS | |
| | KPGAVSTPVKHADDHTPKTVEEVTVERNEKQTPSLPEPKPVY | |
| | AQVGQPDVDLPVSPSDGVLPNSTHEDGILRPSMKLVKFRKGD | |
| | SVGLRLAGGNDVGIFVAGVLEDSPAAKEGLEEGDQILRVNNVD | |
| | FTNIIREEAVLFLLDLPKGEEVTILAQKKKDVYRRIVESDVGDSF | |
| | YIRTHFEYEKESPYGLSFNKGEVFRVVDTLYNGKLGSWLAIRIG | |
| | KNHKEVERGIIPNKNRAEQLASVQYTLPKTAGGDRADFWRFR | |
| | GLRSSKRNLRKSREDLSAQPVQTKFPAYERVVLREAGFLRPV | |
| | TIFGPIADVAREKLAREEPDIYQIAKSEPRDAGTDQRSSGIIRLH | |
| | TIKQIIDQDKHALLDVTPNAVDRLNYAQWYPIVVFLNPDSKQGV | |
| | KTMRMRLCPESRKSARKLYERSHKLRKNNHHLFTTTINLNSM | |
| | NDGWYGALKEAIQQQQNQLVWVSEGKADGATSDDLDLHDDR | |
| | LSYLSAPGSEYSMYSTDSRHTSDYEDTDTEGGAYTDQELDET | |
| | LNDEVGTPPESAITRSSEPVREDSSGMHHENQTYPPYSPQAQ | |
| | PQPIHRIDSPGFKPASQQKAEASSPVPYLSPETNPASSTSAVN | |
| | HNVNLTNVRLEEPTPAPSTSYSPQADSLRTPSTEAAHIMLRDQ | |
| | EPSLSSHVDPTKVYRKDPYPEEMMRQNHVLKQPAVSHPGHR | |
| | PDKEPNLTYEPQLPYVEKQASRDLEQPTYRYESSSYTDQFSR | |
| | NYEHRLRYEDRVPMYEEQWSYYDDKQPYPSRPPFDNQHSQ | |
| | DLDSRQHPEESSERGYFPRFEEPAPLSYDSRPRYEQAPRASA | |
| | LRHEEQPAPGYDTHGRLRPEAQPHPSAGPKPAESKQYFEQY | |
| | SRSYEQVPPQGFTSRAGHFEPLHGAAAVPPLIPSSQHKPEAL | |
| | PSNTKPLPPPPTQTEEEEDPAMKPQSVLTRVKMFENKRSASL | |
| | ETKKDVNDTGSFKPPEVASKPSGAPIIGPKPTSQNQFSEHDKT | |
| | LYRIPEPQKPQLKPPEDIVRSNHYDPEEDEEYYRKQLSYFDRR | |
| | SFENKPPAHIAASHLSEPAKPAHSQNQSNFSSYSSKGKPPEA | |
| | DGVDRSFGEKRYEPIQATPPPPPLPSQYAQPSQPVTSASLHIH | |
| | SKGAHGEGNSVSLDFQNSLVSKPDPPPSQNKPATFRPPNRE | |
| | DTAQAAFYPQKSFPDKAPVNGTEQTQKTVTPAYNRFTPKPYT | |
| | SSARPFERKFESPKFNHNLLPSETAHKPDLSSKTPTSPKTLVK | |
| | SHSLAQPPEFDSGVETFSIHAEKPKYQINNISTVPKAIPVSPSA | |
| | VEEDEDEDGHTVVATARGIFNSNGGVLSSIETGVSIIIPQGAIPE | |
| | GVEQEIYFKVCRDNSILPPLDKEKGETLLSPLVMCGPHGLKFL | |
| | KPVELRLPHCASMTPDGWSFALKSSDSSSGDPKTWQNKCLP | |
| | GDPNYLVGANCVSVLIDHF | |
| 10 | cgcctgagtt gcccgegacg gctctgcccg ccgacggcac gtctctcggc | Mouse Tjp 1 DNA |
| | ggccgcgcgt tccggggaag ttacgtgcgg gagcaggctt tggaggagac | sequence |
| | gcccgagggt gtaggggaca gccggaggcc cgggtactgc ggagcggcga | (GenBank: |
| | gccggcggag ggeggcggag gccgagcagg cggccgggtg tgccccgcgg | D14340.1) |
| | agaagcccgg gcggggcgga cgcttcccgg acttttgtcc cacttgaatc | (Residues in |
| | ccctccccgt gggccgggcc tttccggcct cccccgcccc tgccccgctc | italics |
| | gtccccggga gatgtttatg cggacggtgg cgtgaggagc gggcgggccg | correspond to the |
| | gcggcgcgga gtttcgggtc cgaggagctt cgcgcggcgc ggagagagac | open reading |
| | aagatgtccg ccagggccgc ggccgctaag agcacagcaa tggaggaaac | frame.) |
| | agctatatgg gaacagcaca cagtgacgct tcacagggct cctgggtttg | |
| | gatttggaat tgcaatctct ggtggaagag ataatcctca ttttcagagt | |
| | ggggaaacct ccatagtgat ttctgatgtg ttaaaaggag ggccagctga | |
| | aggacagcta caggaaaatg accgagttgc aatggttaac ggagtttcaa | |
| | tggataacgt tgaacatgct tttgctgttc agcagctaag gaagagtggg | |
| | aaaaacgcaa aaattactat ccgaaggaag aagaaagttc agatccctgt | |
| | aagtcaccca gatcctgagc cggtgtctga taatgaagac gatagttatg | |
| | acgaagaagt gcatgaccca agagctggcc gcggtgcttt agcgaacaga | |
| | aggagcgaga agagctgggc aagggatagg agtgcaagca gggagaggag | |
| | cctgtcccct cgctcggaca ggcggtccgt ggcctccagt cagcccgcaa | |
| | agcccaccaa ggtcacactg gtgaagtctc ggaaaaatga agaatatggt | |
| | cttcgaccgg ccagccacat atttgtaaag gaaatttcac aagatagttt | |
| | ggcagcaaga gatggtgaca ttcaagaagg ggatgttgtc ttgaagataa | |
| | atggtactgt gacagaaaat atgtcattga cagatgcaaa aacactgata | |
| | gaaaggtcta aaggcaagtt aaaaatggta gtgcaaagag atgagcgggc | |
| | taccttactg aacgtccctg acctttcgga tagtatccat tctgctaatg | |
| | cctcggaaag agatgacatt tcagaaattc agtcactagc gtcagaccat | |
| | tcaggtcgct cgcatgacag gccaccccgc cgcagccagt cacgatctcc | |
| | tgaccaacgt tcagagccct ccgatcatto cacgcagtct ccacagcagc | |
| | ccagcaatgg cagtctccgg agcagagagg aagagcgaat gtctaaacct | |
| | ggggccatct caactcctgt aaaacatgta gacgatcato cacccaaagc | |
| | agtggaagaa gttacagttg agaaaaatga gaagcagacg cccactcttc | |
| | cagaaccgaa acctgtgtat gctcaagttg gacaaccaga tgtggattta | |
| | cccgtcagcc cttctgatgg tgctctgcct aattcagctc atgaagacgg | |
| | gatacttagg cccagcatga aactggtaaa attcagaaaa ggagatagtg | |
| | tgggtttgcg actagctggt ggaaatgatg tcggaatatt tgtagctggc | |
| | gttctagaag atagccctgc agccaaagaa ggcttagagg aaggtgatca | |
| | aattctcagg gtgaacaatg tagatttcac aaatatcata agggaagagg | |
| | ccgtcctttt cctccttgac ctccctaaag gtgaagaagt gaccatactg | |
| | gctcagaaga agaaggacgt ttatcgccgc attgtagaat cagatgtagg | |
| | agattcattc tatattagaa cgcattttga atatgaaaaa gaatctcctt | |
| | acggacttag ttttaacaaa ggagaggtgt tccgggtcgt ggatacttta | |
| | tacaatggaa agctgggctc ttggcttgcc attcgaattg gcaaaaatca | |
| | taaggaggta gaacgaggca tcatcccaaa taagaacaga gctgaacagt | |
| | tagccagtgt acagtacaca cttccaaaga cagcgggtgg tgatcgggca | |
| | gacttctgga ggtttcgagg tcttcgcagc tccaagagaa atcttcgaaa | |
| | aagcagagag gacttgtcag ctcagccagt tcaaactaag ttcccagctt | |
| | atgaaagggt tgttcttcga gaagctggat tcctaagacc tgtaaccatc | |
| | tttggaccaa tagctgatgt tgccagagaa aagttggcaa gagaggagcc | |
| | agatatctat cagattgcaa aaagtgaact acgagacgct gggactgacc | |
| | atcgtagctc tggcatcatt cgccttcata caataaagca aatcatagat | |
| | caggataaac atgctttatt agatgtaacg ccaaatgcgg ttgatcgtct | |
| | taattatgcg cagtggtatc caattgttgt gttccttaac cctgactcta agcaaggtgt | |
| | aaaaacaatg aggatgaggt tgtgtccgga gtctcggaaa agcgccagga | |
| | agctatatga acgctctcat aagcttogta agaacaatca ccatctcttc | |
| | acaactacaa ttaacttaaa ctcaatgaat gatggttggt acggtgccct | |
| | gaaagaagcg attcagcagc aacagaacca gctggtgtgg gtctctgagg | |
| | ggaaggcgga tggtgctaca agtgatgacc ttgatttgca tgacgatcgt | |
| | ctgtcctacc tgtcagcccc aggtagtgag tactcaatgt atagcacgga | |
| | cagtagacac acttctgact atgaagacac agatacagaa ggcggggcct | |
| | acactgatca agaactagat gaaactctta atgatgaggt ggggactccc | |
| | ccggagtctg ccattacacg gtcctctgag cctgtaagag aggattcctc | |
| | tggaatgcat catgaaaacc agacataccc tccttactca ccacaagcgc | |
| | agccacaagc tattcataga atagactccc ctggacttaa gccagcctct | |
| | caacagaaag cagaagcctc atctccagtc ccttaccttt cgcctgaaac | |
| | aaccccagca tcatcagcct ctgcagttaa tcataatgtc agtgtaacta | |
| | atgtcagcct ggaggagcct gccccagccc ctcccacctc gcacgcatca | |
| | cagcctggtt gtttaggagc accaagtgct gaggcagctc acgtaggtct | |
| | cagaggtgaa ggaccaccat tgccgccgca tgcagaccca gcaaaggtgt | |
| | acaggaagga gccatattct gaagaaatga tgagacaaaa ccatatttta | |
| | aaacaaccag ctcttggtca cccagggcag aggccagata aagagccaaa | |
| | tctagcctat gaaccccaac ttccatatat agaaaaacaa gccagcagag | |
| | accttgagca gccgtcatac aggtatgagg tctcaagcta cacagaccag | |
| | ttttctcgga actatgacca tegcctacgg tttgaagatc gaatccctac | |
| | ctatgaagac cagtggtcat attatgatga caaacagccc taccaacctc | |
| | ggccttttga gaatcagcat ccccgagacc tggactccag acaacatccc | |
| | gaagaggctt cagaacgagg ttatttccag cgttttgaag agccagcccc | |
| | tctgtcgtac gacagtagaa cacgctatga gcagctgcct cgaacctcta | |
| | ctctacgaca tgaagagcag ccagcccctg catatgaggt gcacaacagg | |
| | tacaggccag aggcacagcc ctattcttca acaggcccta agtcatctga | |
| | gcccaagcag tactttgacc agtacccgcg aagttatgag caagttccac | |
| | caccaggctt tacctccaaa acaggccatt acgagectct ccatggtgct | |
| | gcagttgtcc ctcctctgat accttcctct caacaaaagc cagaagtcct | |
| | gcgctcggct accaaaccac agcctccacc cccaacccta actgaggagg | |
| | aggaggatcc agcaatgaaa ccacagtctg tgctcaccag agtcaaaatg | |
| | tttgaaaaca aaagatctgc gtctttggag aacaagaaag atgtgaatga | |
| | cactgccagc ttcaagcctc cggaagtage atctaaacct ccaggtgott | |
| | ctcttgctgg ccctaaacct gtccctcaga gtcagtttag tgagcacgac | |
| | aaaacgctct acaggctccc agagcctcag aaacctcaag tgaagccacc | |
| | cgaagatatt gttcgatcaa atcattacga ccctgaagag gatgaagaat | |
| | attaccggaa acagctctcc tactttgacc gaagaagttt tgagagcaag | |
| | ccttctgcac atcttcctgc tggccatcac tcagagectg ctaagccagt | |
| | ccattctcag agccagccca acttccctag ttactcttca aagggaaaac | |
| | ccgaaactga tgctgtggat agatcattca gtgagaaacg ttatgatcca | |
| | gcccaggcca cgcctcctcc tcctccgttg ccctcacagt acagccagcc | |
| | agctccacct ctgtccagct cttctctcca catacattcc aagggcgccc | |
| | agggtgaagg caactcagta tcattggatt ttcagaactc atatatgtcc | |
| | aaaccagacc cacccccatc tcagagtaaa ccagcaactt tcagaccacc | |
| | aactcgagaa gacccccctc agactttcta tccgcagaaa agtttcccag | |
| | acaaagctcc agttaacgga gctgagcaga ctcagaaaac catcactccg | |
| | gtgtacaatc gattcacacc aaagccgtac acaagttctg cccggccatt | |
| | tgaacgcaaa tttgaaagtc cgaagttcaa ccataatctt ctgccaagtg | |
| | aaactgtaca taaacctgaa ttgtcttcaa aaactcccac ttccccaaaa | |
| | actcttatga aagctcatag ttcaacacag cctccagagt ttggcagtgg | |
| | agttgaaact ttctctgttc acacagataa gcctaaatat caaatgaata | |
| | atatcagcac catgcctaaa gctgtccctg tgagtccttc agctgtggaa | |
| | gaagatgaag atgaggatgg tcatactgta gtggctacag ctcgtggcat | |
| | ttttaacagc aatggtggtg tgttgagttc catagaaacc ggtgttagta taattatccc | |
| | acaaggagcc attcctgaag gaattgagca agaaatctat ttcaaagtct | |
| | gcagagacaa tagcattctc ccacctttag ataaagagaa aggtgaaact | |
| | ctgctgagcc ccctagtgat gtgtgggccc catggcctca agttcctgaa | |
| | gcccgtggag ctacgcttgc cacactgtga ccctaaaacc tggcaaaaca | |
| | agtgtcttcc tggagatccg aattaccttg ttggagccaa ctgtgtttct gtcctgattg | |
| | accactttta attcttagtg tataggaact ggactaagca atgtgaacgt | |
| | ggattgaact tactaaatct aaatggaacc actctaatga gtattatatt ttcttagaat | |
| | ttatactgca attggtagta ttaagcattt gttggaactg atgaaggtta | |
| | gcgagcatgc ccctgagcca cggtcagaaa gcatgctaca agctatgtgt | |
| | tattgagtga agaactgtca ggcattggct agaggttcaa agatattttt gctttgtaat | |
| | gatttttgta cttttttatg gtcactgctt aacttcacat actgatttcc gttaaaaata | |
| | ccagccagta aatgggggtg catttgagtt ctgttctttc caaagtacac | |
| | tcaaagttta ttatggcctt ggcctagcat acacatttta ttttattata catgaggtaa | |
| | tgtgcacaca ttttttacaa atgcacctgg aatatataac cagtatagtg | |
| | gatttaacag aaatgtacag cagggggatt tataacttgg ggagggaggg | |
| | tcaaatgaag acaattactt attgtatatg aaaacacatt tcttttaggg | |
| | aaggacacca aagcatgtga gcccggttca tggcctctga atctataaat | |
| | taacatatca ccacagacat gtaaccagca ggaatgcctt accctagtgg | |
| | gtttaattct tccatcattt cgctgtgtat tactaagttt ttatgagttc tgtgcatatc | |
| | tagatactgt accatggaaa agactgagta gattgtggac ttgatggttt | |
| | cagaaatgtg caaattacct ggaagaaaat aatggtgtga cttaaaatat | |
| | ttttttgaca gggggagtgg cagtcttctg ttctttgggc tgtgcatctg agatgtttga | |
| | tgctttcagg acgcatgtac ataatgcgtg cataccactt ttgttcttgg tttgtaaatt | |
| | aacttttata aactttacct ttttatacat aaacagaacc aagtttctaa ggctaccttt | |
| | gtattctctc ctgtacctct tgagccttga actttgacct ctgcagcaat aaagcagcat | |
| | ttctacacat acaaggtcat ttttctaaga aaaagaatgc acagagttgt tacattttta | |
| | agtgctgcat ttaaaagata cagttactct agtttgatta aattcttgca aagtatccct | |
| | tctgtaaaat ttgtgataca atactgtgcc ctaaagtgta tttttttact aatagacagt | |
| | ttattatggc acatcagcac gatttctgtt tagataatac accactacat totgttaata | |
| | atcagtaggt gtgaaaggat ttcttttgtg gttattaaaa aaaaatctca aatttctaaa | |
| | tctgcaagaa taaaactttt taaaat | |
| 11 | atgtccg ccagggccgc ggccgctaag agcacagcaa tggaggaaac | Mouse Tjp1 DNA |
| | agctatatgg gaacagcaca cagtgacgct tcacagggct cctgggtttg | sequence-open |
| | gatttggaat tgcaatctct ggtggaagag ataatcctca ttttcagagt | reading frame |
| | ggggaaacct ccatagtgat ttctgatgtg ttaaaaggag ggccagctga | (residues 404-5641 |
| | aggacagcta caggaaaatg accgagttgc aatggttaac ggagtttcaa | of SEQ ID NO: 10) |
| | tggataacgt tgaacatgct tttgctgttc agcagctaag gaagagtggg | |
| | aaaaacgcaa aaattactat ccgaaggaag aagaaagttc agatccctgt | |
| | aagtcaccca gatcctgago cggtgtctga taatgaagac gatagttatg | |
| | acgaagaagt gcatgaccca agagctggcc gcggtgcttt agcgaacaga | |
| | aggagcgaga agagctgggc aagggatagg agtgcaagca gggagaggag | |
| | cctgtcccct cgctcggaca ggeggtccgt ggcctccagt cagcccgcaa | |
| | agcccaccaa ggtcacactg gtgaagtctc ggaaaaatga agaatatggt | |
| | cttcgaccgg ccagccacat atttgtaaag gaaatttcac aagatagttt | |
| | ggcagcaaga gatggtgaca ttcaagaagg ggatgttgtc ttgaagataa | |
| | atggtactgt gacagaaaat atgtcattga cagatgcaaa aacactgata | |
| | gaaaggtcta aaggcaagtt aaaaatggta gtgcaaagag atgagcgggc | |
| | taccttactg aacgtccctg acctttcgga tagtatccat tctgctaatg | |
| | cctoggaaag agatgacatt tcagaaattc agtcactagc gtcagaccat | |
| | tcaggtcgct cgcatgacag gccaccccgc cgcagccagt cacgatetcc | |
| | tgaccaacgt tcagagccct ccgatcatto cacgcagtct ccacagcage | |
| | ccagcaatgg cagtctccgg agcagagagg aagagcgaat gtctaaacct | |
| | ggggccatct caactcctgt aaaacatgta gacgatcato cacccaaagc | |
| | agtggaagaa gttacagttg agaaaaatga gaagcagacg cccactcttc | |
| | cagaaccgaa acctgtgtat gctcaagttg gacaaccaga tgtggattta | |
| | cccgtcagcc cttctgatgg tgctctgcct aattcagctc atgaagacgg | |
| | gatacttagg cccagcatga aactggtaaa attcagaaaa ggagatagtg | |
| | tgggtttgcg actagctggt ggaaatgatg toggaatatt tgtagctggc | |
| | gttctagaag atagccctgc agccaaagaa ggcttagagg aaggtgatca | |
| | aattctcagg gtgaacaatg tagatttcac aaatatcata agggaagagg | |
| | ccgtcctttt cctccttgac ctccctaaag gtgaagaagt gaccatactg | |
| | gctcagaaga agaaggacgt ttatcgccgc attgtagaat cagatgtagg | |
| | agattcattc tatattagaa cgcattttga atatgaaaaa gaatctcctt | |
| | acggacttag ttttaacaaa ggagaggtgt tccgggtcgt ggatacttta | |
| | tacaatggaa agctgggctc ttggcttgcc attcgaattg gcaaaaatca | |
| | taaggaggta gaacgaggca tcatcccaaa taagaacaga gctgaacagt | |
| | tagccagtgt acagtacaca cttccaaaga cagcgggtgg tgatcgggca | |
| | gacttctgga ggtttcgagg tottcgcagc tocaagagaa atcttcgaaa | |
| | aagcagagag gacttgtcag ctcagccagt tcaaactaag ttcccagott | |
| | atgaaagggt tgttcttcga gaagctggat tcctaagacc tgtaaccatc | |
| | tttggaccaa tagctgatgt tgccagagaa aagttggcaa gagaggagcc | |
| | agatatctat cagattgcaa aaagtgaact acgagacgct gggactgacc | |
| | atcgtagctc tggcatcatt cgccttcata caataaagca aatcatagat | |
| | caggataaac atgctttatt agatgtaacg ccaaatgcgg ttgatcgtct | |
| | taattatgcg cagtggtatc caattgttgt gttccttaac cctgactcta agcaaggtgt | |
| | aaaaacaatg aggatgaggt tgtgtccgga gtctcggaaa agcgccagga | |
| | agctatatga acgctctcat aagcttogta agaacaatca ccatctcttc | |
| | acaactacaa ttaacttaaa ctcaatgaat gatggttggt acggtgccct | |
| | gaaagaagcg attcagcage aacagaacca gctggtgtgg gtctctgagg | |
| | ggaaggcgga tggtgctaca agtgatgacc ttgatttgca tgacgatcgt | |
| | ctgtcctacc tgtcagcccc aggtagtgag tactcaatgt atagcacgga | |
| | cagtagacac acttctgact atgaagacac agatacagaa ggcggggcct | |
| | acactgatca agaactagat gaaactotta atgatgaggt ggggactccc | |
| | ccggagtctg ccattacacg gtcctctgag cctgtaagag aggattcctc | |
| | tggaatgcat catgaaaacc agacataccc tccttactca ccacaagcgc | |
| | agccacaagc tattcataga atagactocc ctggacttaa gccagcctct | |
| | caacagaaag cagaagcctc atctccagtc ccttaccttt cgcctgaaac | |
| | aaccccagca tcatcagcct ctgcagttaa tcataatgto agtgtaacta | |
| | atgtcagcct ggaggagcct gccccagccc ctoccaccto gcacgcatca | |
| | cagcctggtt gtttaggagc accaagtgct gaggcagctc acgtaggtot | |
| | cagaggtgaa ggaccaccat tgccgccgca tgcagaccca gcaaaggtgt | |
| | acaggaagga gccatattct gaagaaatga tgagacaaaa ccatatttta | |
| | aaacaaccag ctcttggtca cccagggcag aggccagata aagagccaaa | |
| | tctagcctat gaaccccaac ttccatatat agaaaaacaa gccagcagag | |
| | accttgagca gccgtcatac aggtatgagg tctcaagcta cacagaccag | |
| | ttttctcgga actatgacca tegcctacgg tttgaagato gaatccctac | |
| | ctatgaagac cagtggtcat attatgatga caaacagccc taccaacctc | |
| | ggccttttga gaatcagcat ccccgagacc tggactccag acaacatccc | |
| | gaagaggctt cagaacgagg ttatttccag cgttttgaag agccagcccc | |
| | tctgtogtac gacagtagaa cacgctatga gcagctgcct cgaacctcta | |
| | ctctacgaca tgaagagcag ccagcccctg catatgaggt gcacaacagg | |
| | tacaggccag aggcacagcc ctattottca acaggcccta agtcatctga | |
| | gcccaagcag tactttgacc agtaccegcg aagttatgag caagttccac | |
| | caccaggctt tacctccaaa acaggccatt acgagectct ccatggtgct | |
| | gcagttgtcc ctcctctgat accttcctct caacaaaagc cagaagtcct | |
| | gegctcggct accaaaccac agcctccacc cccaacccta actgaggagg | |
| | aggaggatcc agcaatgaaa ccacagtctg tgctcaccag agtcaaaatg | |
| | tttgaaaaca aaagatctgc gtctttggag aacaagaaag atgtgaatga | |
| | cactgccagc ttcaagcctc cggaagtage atctaaacct ccaggtgott | |
| | ctcttgctgg ccctaaacct gtccctcaga gtcagtttag tgagcacgac | |
| | aaaacgctct acaggetccc agagcctcag aaacctcaag tgaagccacc | |
| | cgaagatatt gttcgatcaa atcattacga ccctgaagag gatgaagaat | |
| | attaccggaa acagctctcc tactttgacc gaagaagttt tgagagcaag | |
| | ccttctgcac atcttcctgc tggccatcac tcagagcctg ctaagccagt | |
| | ccattctcag agccagccca acttccctag ttactcttca aagggaaaac | |
| | ccgaaactga tgctgtggat agatcattca gtgagaaacg ttatgatcca | |
| | gcccaggcca cgcctcctcc tectccgttg coctcacagt acagccagcc | |
| | agctocacct ctgtccagct cttctctcca catacattce aagggcgccc | |
| | agggtgaagg caactcagta tcattggatt ttcagaactc | |
| | atatatgtcc aaaccagacc cacccccatc tcagagtaaa ccagcaactt | |
| | tcagaccacc aactegagaa gaccccccto agactttcta tccgcagaaa | |
| | agtttcccag acaaagctcc agttaacgga gctgagcaga ctcagaaaac | |
| | catcactocg gtgtacaatc gattcacace aaagccgtac acaagttctg | |
| | cccggccatt tgaacgcaaa tttgaaagtc cgaagttcaa ccataatctt | |
| | ctgccaagtg aaactgtaca taaacctgaa ttgtcttcaa aaactcccac | |
| | ttccccaaaa actcttatga aagctcatag ttcaacacag cctccagagt | |
| | ttggcagtgg agttgaaact ttctctgttc acacagataa gcctaaatat | |
| | caaatgaata atatcagcac catgcctaaa gctgtccctg tgagtccttc | |
| | agctgtggaa gaagatgaag atgaggatgg tcatactgta gtggctacag | |
| | ctcgtggcat ttttaacagc aatggtggtg tgttgagttc catagaaacc | |
| | ggtgttagta taattatccc acaaggagcc attcctgaag gaattgagca | |
| | agaaatctat ttcaaagtct gcagagacaa tagcattoto ccacctttag | |
| | ataaagagaa aggtgaaact ctgctgagcc ccctagtgat gtgtgggccc | |
| | catggcctca agttcctgaa gcccgtggag ctacgcttgc cacactgtga | |
| | ccctaaaacc tggcaaaaca agtgtcttcc tggagatccg aattaccttg | |
| | ttggagccaa ctgtgtttct gtcctgattg accactttta a | |
| 12 | MSARAAAAKSTAMEETAIWEQHTVTLHRAPGFGFGIAISGGRD | Mouse Tjp1 amino |
| | NPHFQSGETSIVISDVLKGGPAEGQLQENDRVAMVNGVSMDN | acid sequence |
| | VEHAFAVQQLRKSGKNAKITIRRKKKVQIPVSHPDPEPVSDNE | |
| | DDSYDEEVHDPRAGRGALANRRSEKSWARDRSASRERSLSP | |
| | RSDRRSVASSQPAKPTKVTLVKSRKNEEYGLRPASHIFVKEIS | |
| | QDSLAARDGDIQEGDVVLKINGTVTENMSLTDAKTLIERSKGK | |
| | LKMVVQRDERATLLNVPDLSDSIHSANASERDDISEIQSLASDH | |
| | SGRSHDRPPRRSQSRSPDQRSEPSDHSTQSPQQPSNGSLRS | |
| | REEERMSKPGAISTPVKHVDDHPPKAVEEVTVEKNEKQTPTL | |
| | PEPKPVYAQVGQPDVDLPVSPSDGALPNSAHEDGILRPSMKL | |
| | VKFRKGDSVGLRLAGGNDVGIFVAGVLEDSPAAKEGLEEGDQ | |
| | ILRVNNVDFTNIIREEAVLFLLDLPKGEEVTILAQKKKDVYRRIVE | |
| | SDVGDSFYIRTHFEYEKESPYGLSFNKGEVFRVVDTLYNGKLG | |
| | SWLAIRIGKNHKEVERGIIPNKNRAEQLASVQYTLPKTAGGDR | |
| | ADFWRFRGLRSSKRNLRKSREDLSAQPVQTKFPAYERVVLRE | |
| | AGFLRPVTIFGPIADVAREKLAREEPDIYQIAKSELRDAGTDHR | |
| | SSGIIRLHTIKQIIDQDKHALLDVTPNAVDRLNYAQWYPIVVFLN | |
| | PDSKQGVKTMRMRLCPESRKSARKLYERSHKLRKNNHHLFTT | |
| | TINLNSMNDGWYGALKEAIQQQQNQLVWVSEGKADGATSDD | |
| | LDLHDDRLSYLSAPGSEYSMYSTDSRHTSDYEDTDTEGGAYT | |
| | DQELDETLNDEVGTPPESAITRSSEPVREDSSGMHHENQTYP | |
| | PYSPQAQPQAIHRIDSPGLKPASQQKAEASSPVPYLSPETTPA | |
| | SSASAVNHNVSVTNVSLEEPAPAPPTSHASQPGCLGAPSAEA | |
| | AHVGLRGEGPPLPPHADPAKVYRKEPYSEEMMRQNHILKQPA | |
| | LGHPGQRPDKEPNLAYEPQLPYIEKQASRDLEQPSYRYEVSS | |
| | YTDQFSRNYDHRLRFEDRIPTYEDQWSYYDDKQPYQPRPFE | |
| | NQHPRDLDSRQHPEEASERGYFQRFEEPAPLSYDSRTRYEQ | |
| | LPRTSTLRHEEQPAPAYEVHNRYRPEAQPYSSTGPKSSEPKQ | |
| | YFDQYPRSYEQVPPPGFTSKTGHYEPLHGAAVVPPLIPSSQQ | |
| | KPEVLRSATKPQPPPPTLTEEEEDPAMKPQSVLTRVKMFENK | |
| | RSASLENKKDVNDTASFKPPEVASKPPGASLAGPKPVPQSQF | |
| | SEHDKTLYRLPEPQKPQVKPPEDIVRSNHYDPEEDEEYYRKQ | |
| | LSYFDRRSFESKPSAHLPAGHHSEPAKPVHSQSQPNFPSYSS | |
| | KGKPETDAVDRSFSEKRYDPAQATPPPPPLPSQYSQPAPPLS | |
| | SSSLHIHSKGAQGEGNSVSLDFQNSYMSKPDPPPSQSKPATF | |
| | RPPTREDPPQTFYPQKSFPDKAPVNGAEQTQKTITPVYNRFT | |
| | PKPYTSSARPFERKFESPKFNHNLLPSETVHKPELSSKTPTSP | |
| | KTLMKAHSSTQPPEFGSGVETFSVHTDKPKYQMNNISTMPKA | |
| | VPVSPSAVEEDEDEDGHTVVATARGIFNSNGGVLSSIETGVSIII | |
| | PQGAIPEGIEQEIYFKVCRDNSILPPLDKEKGETLLSPLVMCGP | |
| | HGLKFLKPVELRLPHCDPKTWQNKCLPGDPNYLVGANCVSVL | |
| | IDHF | |
| 13 | CTT CTC TGA CCC TAC ACA GOT ACC | Primer 1 for |
| | | genotyping |
| 14 | ATC GTG TGG GAA AGA CAA GC | Primer 2 for |
| | | genotyping |
| 15 | CCA TTT GTC CTC ATC TCT TAC TAA C | Yap specific primer |
| | | 1 |
| 16 | GAT TGG GCA CTG TCA ATT AAT GGG CTT | Yap specific primer |
| | | 2 |
| 17 | GGCCCAGAGCAAGAGAGGTATCC | Actin forward |
| | | primer |
| 18 | ACGCACGATTTCCCTCTCAGC | Actin reverse |
| | | primer |
| 19 | AGCACAGCAATGGAGGAAACAGCT | Tjp1 forward primer |
| 20 | CTGGGTGACTTACAGGGATCTGAA | Tjp1 reverse primer |
| 21 | AGCGTGATCAGCGGTTTTG | CK19 forward |
| | | primer |
| 22 | CCTGGTTCTGGCGCTCTATG | CK19 reverse |
| | | primer |
| 23 | GTCATTTGCTCCAAACTGGCG | EPCAM forward |
| | | primer |
| 24 | AGCCCATCGTTGTTCTGGAT | EPCAM reverse |
| | | primer |
| 25 | GACTGGAAGAGCGGAGAGTACTG | Collagen 1A1 |
| | | forward primer |
| 26 | CAGGTCTGACCTGTCTCCATGTT | Collagen 1A1 |
| | | reverse primer |
| 27 | GCAGTGGCTGAACCAAGG | TGFβ forward |
| | | primer |
| 28 | AGCAGTGAGCGCTGAATC | TGFβ reverse |
| | | primer |
| 29 | GGCATCTGGCATCCTCTTGT | TIMP-1 forward |
| | | primer |
| 30 | TAGCCCTTATGACCAGGTCCG | TIMP-1 reverse |
| | | primer |
| 31 | GCGGTCCTCACCATGAGTCC | MMP9 forward |
| | | primer |
| 32 | TAGCGGTACAAGTATGCCTCTGC | MMP9 reverse |
| | | primer |
| 33 | CCTTCGTGACTACTGCCGAGC | α-SMA forward |
| | | primer |
| 34 | TTCGTGGATGCCCGCTGACT | α-SMA reverse |
| | | primer |
| 35 | GCAGAAGAAGCCACGCTATG | PdgfRb forward |
| | | primer |
| 36 | CAGGTGGAGTCGTAAGGCAA | PdgfRb reverse |
| | | primer |
| 37 | GAGTCGGCATGAATCGCTG | Pdgfb forward |
| | | primer |
| 38 | TCAAAGGAGCGGATGGAGTG | Pdgfb reverse |
| | | primer |
| 39 | ACATTAAGAAGGGCAAAAAGTGCA | CTGF forward |
| | | primer |
| 40 | AACTTAGCCCTGTATGTCTTCACA | CTGF reverse |
| | | primer |
| 41 | CACGTCGTAGCAAACCACC | TNFα forward |
| | | primer |
| 42 | GGTGAGGAGCACGTAGTC | TNFα reverse |
| | | primer |
| 43 | CCTCTGGTCTTCTGGAGTACC | IL-6 forward primer |
| 44 | ACTCCTTCTGTGACTCCAGC | IL-6 reverse primer |
| 45 | TGCCACCTTTTGACAGTGATG | IL-1b forward |
| | | primer |
| 46 | ATGTGCTGCTGCGAGATTTG | IL-1b reverse |
| | | primer |
| 47 | AACATGCAACCTGCCACAAC | F4/80 forward |
| | | primer |
| 48 | TTCACAGGATTCGTCCAGGC | F4/80 reverse |
| | | primer |
| 49 | ACACTTTCACTCCAATCGTCC | Osteopontin |
| | | forward primer |
| 50 | TGCCCTTTCCGTTGTTGTCC | Osteopontin |
| | | reverse primer |
| 51 | GCAGCCAGATTGGCTCTTATTTT | CD11b forward |
| | | primer |
| 52 | GCCCTCGAGGCAAGGGA | CD11b reverse |
| | | primer |
| 53 | GGTGGTGAGAGCTTGAAAATGA | Cyp7a1 forward |
| | | primer |
| 54 | GTGGTTCTTGGAGGTGCCTTT | Cyp7a1 reverse |
| | | primer |
| 55 | CTATGGAAGCCCTGCGTGAC | Cyp7b1 forward |
| | | primer |
| 56 | GAGCACAGCCTCAGAACCTC | Cyp7b1 reverse |
| | | primer |
| 57 | TTGCAAATGCTGCCTCAACC | Cyp8b1 forward |
| | | primer |
| 58 | TAACAGTTCGCACACATGGCT | Cyp8b1 reverse |
| | | primer |
| 59 | GAGACAGGAGGGCAAGTACC | Cyp27a1 forward |
| | | primer |
| 60 | TAAGGCATCCGTGTAGAGCG | Cyp27a1 reverse |
| | | primer |
| 61 | GCATTACCAAGAACGCCGTG | FXR forward |
| | | primer |
| 62 | CTGGATTTCAGTTAACAAACCTGT | FXR reverse |
| | | primer |
| 63 | CCAGGCCAGTAGAGTGGTAGC | Shp-1 forward |
| | | primer |
| 64 | GGCAGGCACGTACAAACTC | Shp-1 reverse |
| | | primer |
| 65 | TACGTCCTCAAGGCAGGCATGATC | NTCP forward |
| | | primer |
| 66 | GTAGCCAGTAAGTGTGGTGTCATG | NTCP reverse |
| | | primer |
| 67 | ACGCAAGATCCAACAGAGTGT | OATP-1 forward |
| | | primer |
| 68 | GGGCCAACAATCTTCCCCAT | OATP-1 reverse |
| | | primer |
| 69 | TGAGTTCAGGACACCAAGACTG | OATP-2 forward |
| | | primer |
| 70 | ACCGCCTACCCTATGCCTT | OATP-2 reverse |
| | | primer |
| 71 | CATCTGTAGGGTTGTTGAGTGA | ABCB11 forward |
| | | primer |
| 72 | CAAAGAAGCCAACTCGAGCG | ABCB11 reverse |
| | | primer |
| 73 | ACGGCGACTTTGAACTAGGC | ABCB4 forward |
| | | primer |
| 74 | CCAGTCAGAGTATCGGAACAGT | ABCB4 reverse |
| | | primer |
| 75 | GCAACTCTACTTTTTGGAATCTCT | ABCC2 forward |
| | | primer |
| 76 | CCAAGAGCCAAAGAAAGCCC | ABCC2 reverse |
| | | primer |
| 77 | TGAGGATGCGGTCCTACTGA | ABCC3 forward |
| | | primer |
| 78 | GGCCGTGGGGTCAGAAATAA | ABCC3 reverse |
| | | primer |
| 79 | GACCATGAAGCCTTGCAAAA | ABCC4 forward |
| | | primer |
| 80 | TTGGGCATAGTCCGGTTCTG | ABCC4 reverse |
| | | primer |
| 81 | ACCTGGGTGCTCCTAGCAA | Cyp3a11 forward |
| | | primer |
| 82 | AAATGGCAGAGGTTTGGGC | Cyp3a11 reverse |
| | | primer |
| 83 | CAGACCCGGAGTTACCCAAAG | CAR forward |
| | | primer |
| 84 | AAACCGACTTTGGAGCCGAG | CAR reverse |
| | | primer |
| 85 | GGCCTGACCTTGGCTCATAG | PXR forward |
| | | primer |
| 86 | ACATCTGTGTGTCCTAGACTGT | PXR reverse |
| | | primer |
| 87 | TGTGTGTGTTCGGTCCCTC | Ugt1a1 forward |
| | | primer |
| 88 | GCTTCAGGTGCTATGACCAC | Ugt1a1 reverse |
| | | primer |
| 89 | CCTCAAAGGAAATGTTCTATTCGGA | Slut2a1 forward |
| | | primer |
| 90 | TCCAGCTCATCTGGCCCTAA | Slut2a1 reverse |
| | | primer |
| 91 | ATTTAGTAAGTTCAATCCACG | Complementary |
| | | sequence of SEQ |
| | | ID NO: 4 |
| 92 | AACTTGCTCATAACTTCGCGG | Complementary |
| | | sequence of SEQ |
| | | ID NO: 5 |
| 93 | AAATTTGCGTTCAAATGGCCG | Complementary |
| | | sequence of SEQ |
| | | ID NO: 6 |
| 94 | UGAAACUCCGUUAACCAUUGC | siRNA Tjp1 |
| | | sequence 1; |
| | | targets residues |
| | | 634-654 of mouse |
| | | Tjp1 cDNA |
| | | sequence |
| | | (NM_009386.2) or |
| | | residues 667-687 |
| | | of human Tjp1 |
| | | cDNA sequence |
| | | (NM_003257.5) |
| 95 | AUUGAAACUCCGUUAACCAUU | siRNA Tjp1 |
| | | sequence 2; |
| | | targets residues |
| | | 637-657 of mouse |
| | | Tjp1 cDNA |
| | | sequence |
| | | (NM_009386.2) or |
| | | residues 668-688 |
| | | of human Tjp1 |
| | | cDNA sequence |
| | | (NM_003257.5) |
| 96 | ACUAUCUUGUGAAAUUUCCUU | siRNA Tjp1 |
| | | sequence 3; |
| | | targets residues |
| | | 1033-1053 of |
| | | mouse Tjp1 cDNA |
| | | sequence |
| | | (NM_009386.2) or |
| | | residues 1065- |
| | | 1085 of human |
| | | Tjp1 cDNA |
| | | sequence |
| | | (NM_003257.5) |
| 97 | AAAUCAAGGUCAUCACUUGUA | siRNA Tjp1 |
| | | sequence 4; |
| | | targets residues |
| | | 2832-2852 of |
| | | mouse Tjp1 cDNA |
| | | sequence |
| | | (NM_009386.2) or |
| | | residues 2864- |
| | | 2884 of human |
| | | Tjp1 cDNA |
| | | sequence |
| | | (NM_003257.5) |
| 98 | UUCAUCUAGUUCUUGAUCAGU | siRNA Tjp1 |
| | | sequence 5; |
| | | targets residues |
| | | 2968-2988 of |
| | | mouse Tjp1 cDNA |
| | | sequence |
| | | (NM_009386.2) or |
| | | residues 3000- |
| | | 3020 of human |
| | | Tjp1 cDNA |
| | | sequence |
| | | (NM_003257.5) |
| 99 | UAAGAGUUUCAUCUAGUUCUU | siRNA Tjp1 |
| | | sequence 6; |
| | | targets residues |
| | | 2976-2996 of |
| | | mouse Tip1 cDNA |
| | | sequence |
| | | (NM_009386.2) or |
| | | residues 3007- |
| | | 3027 of human |
| | | Tjp1 cDNA |
| | | sequence |
| | | (NM_003257.5) |
| 100 | UUCUCUUUAUCUAAAGGUGGG | siRNA Tjp1 |
| | | sequence 7; |
| | | targets residues |
| | | 5445-5465 of |
| | | mouse Tjp1 cDNA |
| | | sequence |
| | | (NM_009386.2) or |
| | | residues 5486- |
| | | 5506 of human |
| | | Tjp1 cDNA |
| | | sequence |
| | | (NM_003257.5) |
| 101 | AUCUAAACAGAAAUCGUGCUG | siRNA Tjp1 |
| | | sequence 8; |
| | | targets residues |
| | | 6920-6940 of |
| | | mouse Tjp1 cDNA |
| | | sequence |
| | | (NM_009386.2) or |
| | | residues 7004- |
| | | 7024 of human |
| | | Tjp1 cDNA |
| | | sequence |
| | | (NM_003257.5) |
| 102 | UAUCUAAACAGAAAUCGUGCU | SIRNA Tjp1 |
| | | sequence 9; |
| | | targets residues |
| | | 6921-6941 of |
| | | mouse Tjp1 cDNA |
| | | sequence |
| | | (NM_009386.2) or |
| | | residues 7005- |
| | | 7025 of human |
| | | Tjp1 cDNA |
| | | sequence |
| | | (NM_003257.5) |
| 103 | AAUGUAGUGGUGUAUUAUCUA | siRNA Tjp1 |
| | | sequence 10; |
| | | targets residues |
| | | 6936-6956 of |
| | | mouse Tjp1 cDNA |
| | | sequence |
| | | (NM_009386.2) or |
| | | residues 7020- |
| | | 7040 of human |
| | | Tjp1 cDNA |
| | | sequence |
| | | (NM_003257.5) |
| 104 | GCAATGGTTAACGGAGTTTCA | Tjp1 binding site 4 |
| | | -corresponds to |
| | | residues 634-654 |
| | | of mouse Tjp1 |
| | | cDNA sequence |
| | | (NM_009386.2) or |
| | | residues 667-687 |
| | | of human Tjp1 |
| | | cDNA sequence |
| | | (NM_003257.5) |
| 105 | AATGGTTAACGGAGTTTCAAT | Tjp1 binding site 5 |
| | | -corresponds to |
| | | residues 637-657 |
| | | of mouse Tjp1 |
| | | cDNA sequence |
| | | (NM_009386.2) or |
| | | residues 668-688 |
| | | of human Tjp1 |
| | | cDNA sequence |
| | | (NM_003257.5) |
| 106 | AAGGAAATTTCACAAGATAGT | Tjp1 binding site 6 |
| | | -corresponds to |
| | | residues 1033- |
| | | 1053 of mouse |
| | | Tjp1 cDNA |
| | | sequence |
| | | (NM_009386.2) or |
| | | residues 1065- |
| | | 1085 of human |
| | | Tjp1 cDNA |
| | | sequence |
| | | (NM_003257.5) |
| 107 | TACAAGTGATGACCTTGATTT | Tjp1 binding site 7 |
| | | -corresponds to |
| | | residues 2832- |
| | | 2852 of mouse |
| | | Tjp1 cDNA |
| | | sequence |
| | | (NM_009386.2) or |
| | | residues 2864- |
| | | 2884 of human |
| | | Tjp1 cDNA |
| | | sequence |
| | | (NM_003257.5) |
| 108 | ACTGATCAAGAACTAGATGAA | Tjp1 binding site 8 |
| | | -corresponds to |
| | | residues 2968- |
| | | 2988 of mouse |
| | | Tjp1 cDNA |
| | | sequence |
| | | (NM_009386.2) or |
| | | residues 3000- |
| | | 3020 of human |
| | | Tjp1 cDNA |
| | | sequence |
| | | (NM_003257.5) |
| 109 | AAGAACTAGATGAAACTCTTA | Tjp1 binding site 9 |
| | | -corresponds to |
| | | residues 2976- |
| | | 2996 of mouse |
| | | Tjp1 cDNA |
| | | sequence |
| | | (NM_009386.2) or |
| | | residues 3007- |
| | | 3027 of human |
| | | Tjp1 cDNA |
| | | sequence |
| | | (NM_003257.5) |
| 110 | CCCACCTTTAGATAAAGAGAA | Tjp1 binding site |
| | | 10-corresponds to |
| | | residues 5445- |
| | | 5465 of mouse |
| | | Tjp1 cDNA |
| | | sequence |
| | | (NM_009386.2) or |
| | | residues 5486- |
| | | 5506 of human |
| | | Tjp1 cDNA |
| | | sequence |
| | | (NM_003257.5) |
| 111 | CAGCACGATTTCTGTTTAGAT | Tjp1 binding site |
| | | 11-corresponds to |
| | | residues 6920- |
| | | 6940 of mouse |
| | | Tjp1 cDNA |
| | | sequence |
| | | (NM_009386.2) or |
| | | residues 7004- |
| | | 7024 of human |
| | | Tjp1 cDNA |
| | | sequence |
| | | (NM_003257.5) |
| 112 | AGCACGATTTCTGTTTAGATA | Tjp1 binding site |
| | | 12-corresponds to |
| | | residues 6921- |
| | | 6941 of mouse |
| | | Tjp1 cDNA |
| | | sequence |
| | | (NM_009386.2) or |
| | | residues 7005- |
| | | 7025 of human |
| | | Tjp1 cDNA |
| | | sequence |
| | | (NM_003257.5) |
| 113 | TAGATAATACACCACTACATT | Tjp1 binding site |
| | | 13-corresponds to |
| | | residues 6936- |
| | | 6956 of mouse |
| | | Tjp1 cDNA |
| | | sequence |
| | | (NM_009386.2) or |
| | | residues 7020- |
| 114 | gtggccgcct gagttgcccg cgacggctct gcccgccgac ggcacgtctc | 7040 of human |
| | tcggcggccg cgcgttccgg ggaagttacg tgcgggagca ggctttggag | Tjp1 cDNA |
| | gagacgcccg agggtgtagg ggacagccgg aggcccgggt actgcggagc | sequence |
| | ggcgagccgg cggagggcgg cggaggccga gcaggcggcc gggtgtgccc | (NM_003257.5) |
| | cgcggagaag cccgggcggg gcggacgctt cccggacttt tgtcccactt | Mouse Tjp1 cDNA |
| | gaatcccctc cccgtgggcc gggcctttcc ggcctccccc gcccctgccc | sequence |
| | cgctcgtccc cgggagatgt ttatgcggac ggtggcgtga ggagcgggcg | (NM_009386.2) |
| | ggccggcggc gcggagtttc gggtccgagg agcttcgcgc ggcgcggaga | |
| | gagacaagat gtccgccagg gccgcggccg ctaagagcac agcaatggag | |
| | gaaacagcta tatgggaaca gcacacagtg acgcttcaca gggctcctgg | |
| | gtttggattt ggaattgcaa tctctggtgg aagagataat cctcattttc | |
| | agagtgggga aacctccata gtgatttctg atgtgttaaa aggagggcca | |
| | gctgaaggac agctacagga aaatgaccga gttgcaatgg ttaacggagt | |
| | ttcaatggat aacgttgaac atgcttttgc tgttcagcag ctaaggaaga | |
| | gtgggaaaaa cgcaaaaatt actatccgaa ggaagaagaa agttcagatc | |
| | cctgtaagtc acccagatcc tgagccggtg tctgataatg aagacgatag | |
| | ttatgacgaa gaagtgcatg acccaagagc tggccgcggt gctttagcga | |
| | acagaaggag cgagaagagc tgggcaaggg ataggagtgc aagcagggag | |
| | aggagcctgt cccctcgctc ggacaggcgg tccgtggcct ccagtcagcc | |
| | cgcaaagccc accaaggtca cactggtgaa gtctcggaaa aatgaagaat | |
| | atggtcttcg attggccagc catatatttg taaaggaaat ttcacaagat | |
| | agtttggcag caagagatgg taacattcaa gaaggggatg ttgtcttgaa | |
| | gataaatggt actgtgacag aaaatatgtc attgacagat gcaaaaacac | |
| | tgatagaaag gtctaaaggc aagttaaaaa tggtagtgca aagagatgag | |
| | cgggctacct tactgaacgt ccctgacctt tcggatagta tccattctgc taatgcctcg | |
| | gaaagagatg acatttcaga aattcagtca ctagcatcag accattcagg | |
| | tcgctcgcat gacaggccac cccgccgcag ccagtcacga tctcctgacc | |
| | aacgttcaga gccctccgat cattccacgc agtctccaca gcagcccagc | |
| | aatggcagtc tccggagcag agaggaagag cgaatgtcta aacctggggc | |
| | catctcaact cctgtaaaac atgtagacga tcatccaccc aaagcagtgg | |
| | aagaagttac agttgagaaa aatgagaagc agacgcccac tcttccagaa | |
| | ccgaaacctg tgtatgctca agttggacaa ccagatgtgg atttacccgt | |
| | cagcccttct gatggtgctc tgcctaattc agctcatgaa gacgggatac | |
| | ttaggcccag catgaaactg gtaaaattca gaaaaggaga tagtgtgggt | |
| | ttgcgactag ctggtggaaa tgatgtcgga atatttgtag ctggcgttct | |
| | agaagatagc cctgcagcca aagaaggctt agaggaaggt gatcaaattc | |
| | tcagggtgaa caatgtagat ttcacaaata tcataaggga agaggccgtc | |
| | cttttcctcc ttgacctccc taaaggtgaa gaagtgacca tactggctca | |
| | gaagaagaag gacgtttatc gccgcattgt agaatcagat gtaggagatt | |
| | cattctatat tagaacgcat tttgaatatg aaaaagaatc tccttacgga cttagtttta | |
| | acaaaggaga ggtgttccgg gtcgtggata ctttatacaa tggaaagctg | |
| | ggctcttggc ttgccattcg aattggcaaa aatcataagg aggtagaacg | |
| | aggcatcatc cctaataaga acagagctga acagttagcc agtgtacagt | |
| | acacacttcc aaagacagcg ggtggtgatc gggcagactt ctggaggttt | |
| | cgaggtcttc gcagctccaa gagaaatctt cgaaaaagca gagaggactt | |
| | gtcagctcag ccagttcaaa ctaagttccc agcttatgaa agggttgttc | |
| | ttcgagaagc tggattccta agacctgtaa ccatctttgg accaatagct | |
| | gatgttgcca gagaaaagtt ggcaagagag gagccagata tctatcagat | |
| | tgcaaaaagt gaaccacgag atgctgggac tgaccatcgt agctctggca | |
| | tcattcgcct tcatacaata aagcaaatca tagatcagga taaacatgct | |
| | ttattagatg taacgccaaa tgcggttgat cgtcttaatt atgcgcagtg gtatccaatt | |
| | gttgtgttcc ttaaccctga ctctaagcaa ggtgtaaaaa caatgaggat | |
| | gaggttgtgt ccggagtctc ggaaaagcgc caggaagcta tatgaacgct | |
| | ctcataagct tcgtaagaac aatcaccatc tcttcacaac tacaattaac | |
| | ttaaactcaa tgaatgatgg ttggtacggt gccctgaaag aagcgattca | |
| | gcagcaacag aaccagctgg tgtgggtctc tgaggggaag gcggatggtg | |
| | ctacaagtga tgaccttgat ttgcatgacg atcgtctgtc ctacctgtca | |
| | gccccaggta gtgagtactc aatgtatagc acggacagta gacacacttc | |
| | tgactatgaa gacacagata cagaaggcgg ggcctacact gatcaagaac | |
| | tagatgaaac tcttaatgat gaggtgggga ctcccccgga gtctgccatt | |
| | acacggtcct ctgagcctgt aagagaggat tcctctggaa tgcatcatga | |
| | aaaccagaca taccctcctt actcaccaca agcgcagcca caagctattc | |
| | atagaataga ctcccctgga cttaagccag cctctcaaca gaaagcagaa | |
| | gcctcatctc cagtccctta cctttcgcct gaaacaaccc cagcatcatc | |
| | agcctctgca gttaatcata atgtcagtgt aactaatgtc agcctggagg | |
| | agcctgcccc agcccctccc acctcgcacg catcacagcc tggttgttta | |
| | ggagcaccaa gtgctgaggc agctcacgta gtgctcagag gtgaaggacc | |
| | accattgccg ccgcatgcag acccagcaaa ggtgtacagg aaggagccgt | |
| | attctgaaga aatgatgaga caaaaccata ttttaaaaca accagctctt | |
| | ggtcacccag ggcagaggcc agataaagag ccaaatctag cctatgaacc | |
| | ccaacttcca tatatagaaa aacaagccag cagagacctt gagcagccgt | |
| | catacaggta tgaggtctca agctacacag accagttttc tcggaactat | |
| | gaccatcgcc tacggtttga agatcgaatc cctacctatg aagaccagtg | |
| | gtcatattat gatgacaaac agccctacca acctcggcct tttgagaatc | |
| | agcatccccg agacctggac tccagacaac atcccgaaga ggcttcagaa | |
| | cgaggttatt tccagcgttt tgaagagcca gcccctctgt cgtacgacag | |
| | tagaacacgc tatgagcagc tgcctcgaac ctctactcta cgacatgaag | |
| | agcagccagc ccctgcatat gaggtgcaca acaggtacag gccagaggca | |
| | cagccctatt cttcaacagg ccctaagtca tctgagccca agcagtactt | |
| | tgaccagtac ccgcgaagtt atgagcaagt tccaccacca ggctttacct | |
| | ccaaaacagg ccattacgag cctctccatg gtgctgcagt tgtccctcct | |
| | ctgatacctt cctctcaaca aaagccagaa gtcctgccct cggctaccaa | |
| | accacagcct ccacccccaa ccctaactga ggaggaggag gatccagcaa | |
| | tgaaaccaca gtctgtgctc accagagtca aaatgtttga aaacaaaaga | |
| | tctgcgtctt tggagaacaa gaaagatgtg aatgacactg ccagctttaa | |
| | gcctccggaa gtagcatcta aacctccagg tgcttctctt gctggcccta | |
| | aacctgtccc tcagagtcag tttagtgagc acgacaaaac gctctacagg | |
| | ctcccagagc ctcagaaacc tcaagtgaag ccacccgaag atattgttcg | |
| | atcaaatcat tacgaccctg aagaggatga agaatattac cggaaacagc | |
| | tctcctactt tgaccgaaga agttttgaga gcaagccttc tgcacatctt | |
| | cctgctggcc atcactcaga gcctgctaag ccagtccatt ctcagagcca | |
| | gcccaacttc tctagttact cttcaaaggg aaaacccgaa actgatgctg | |
| | tggatagatc attcagtgag aaacgttatg atccagccca ggccacgcct | |
| | cctcctcctc cgttgccctc acagtacagc cagccagctc cacctctgtc | |
| | cagctcttct ctccacatac attccaaggg cgcccagggt gaaggcaact | |
| | cagtatcatt ggattttcag aactcatata tgtccaaacc agacccaccc | |
| | ccatctcaga gtaaaccagc aactttcaga ccaccaactc gagaagaccc | |
| | ccctcagact ttctatccgc agaaaagttt cccagacaaa gctccagtta | |
| | acggagctga gcagactcag aaaaccatca ctccggtgta caatcgattc | |
| | acaccaaagc cgtacacaag ttctgcccgg ccatttgaac gcaaatttga | |
| | aagtccgaag ttcaaccata atcttctgcc aagtgaaact gtacataaac | |
| | ctgaattgtc ttcaaaaact cccacttccc caaaaactct tatgaaagct | |
| | catagttcaa cacagcctcc agagtttgac agtggagttg aaactttctc | |
| | tgttcacaca gataagccta aatatcaaat gaataatatc agcaccatgc | |
| | ctaaagctgt ccctgtgagt ccttcagctg tggaagaaga tgaagatgag | |
| | gatggtcata ctgtagtggc tacagctcgt ggcattttta acagcaatgg | |
| | tggtgtgttg agttccatag aaaccggtgt tagtataatt atcccacaag | |
| | gagccattcc tgaaggaatt gagcaagaaa tctatttcaa agtctgcaga | |
| | gacaatagca ttctcccacc tttagataaa gagaaaggtg aaactctgct | |
| | gagcccccta gtgatgtgtg ggccccatgg cctcaagttc ctgaagcccg | |
| | tggagctacg cttgccacac tgtgacccta aaacctggca aaacaagtgt | |
| | cttcctggag atccgaatta ccttgttgga gccaactgtg tttctgtcct gattgaccac | |
| | ttttaattct tagtgtatag gaactggact aagcaatgtg aacgtggatt gaacttacta | |
| | aatctaaatg gaaccactct aatgagtatt atattttctt agaatttata ctgcaattgg | |
| | tagtattaag catttgttgg aactgatgaa ggttagcgag catgcccctg | |
| | agccacggtc agaaaacatg ctacaagcta tgtgttattg agtgaagaac | |
| | tgtcaggcat tggctagagg ttcaaagata tttttgcttt gtaatgattt ttgtactttt | |
| | ttatggtcac tgcttaactt cacatactga tttccgttaa aaataccagc | |
| | cagtaaatgg gggtgcattt gagttctgtt ctttccaaag tacactcaaa gtttattatg | |
| | gccttggcct agcatacaca ttttatttta ttatgcatga ggtaatgtgc acacattttt | |
| | tacaaatgca cctggaatat ataaccagta tagtggattt aacagaaatg | |
| | tacagcaggg ggatttataa cttggggagg gagggtcaaa tgaagacaat | |
| | tacttattgt atatgaaaac acatttcttt tagggaagga caccaaagca | |
| | tgtgagcccg gttcatggcc tctgaatcta taaattaaca tatcaccaca | |
| | gacatgtaac cagcaggaat gccttaccct agtgggttta attcttccat catttcgctg | |
| | tgtattacta agtttttatg agttctgtgc atatctagat actgtaccat ggaaaagact | |
| | gagtagattg tggacttgat ggtttcagaa atgtgcaaat tacctggaag | |
| | aaaataatgg tgtgacttaa aatatttttt tgacaggggg agtggcagtc ttctgttctt | |
| | tgggctgtgc atctgagatg tttgatgctt tcaggacgca tgtacataat | |
| | gcgtgcatac cacttttgtt cttggtttgt aaattaactt ttataaactt taccttttta | |
| | tacataaaca gaaccaagtt tctaaggcta cctttgtatt ctctcctgta cctcttgagc | |
| | cttgaacttt gacctctgca gcaataaagc agcatttcta cacatacaag gtcatttttc | |
| | taagaaaaag aatgcacaga gttgttacat ttttaagtgc tgcatttaaa | |
| | agatacagtt actctagttt gattaaattc ttgcaaagta tcccttctgt aaaatttgtg | |
| | atacaatact gtgccctaaa gtgtattttt ttactaatag acagtttatt atggcacatc | |
| | agcacgattt ctgtttagat aatacaccac tacattctgt taataatcag taggtgtgaa | |
| | aggatttctt ttgtggttat taaaaaaaaa tctcaaattt ctaaatctgc aagaataaaa | |
| | ctttttaaaa taca | |
| 115 | gtggccgctg agttgccggc gccggctgag ccagcggacg ccgcgttcct | Human Tjp1 cDNA |
| | tggcggccgc cggttcccgg gaagttacgt ggcgaagccg gcttccgagg | sequence |
| | agacgccggg aggccacggg tgctgctgac gggcgggcga ccgggcgagg | (NM_003257.5) |
| | ccgacgtggc cgggctgcga aagctgcggg aggccgagtg ggtggccgcg | |
| | ctcggaggga ggtgccggtc gggcgcgccc cgtggagaag acccgggcgg | |
| | ggcgggcgct tcccggactt ttgtccgagt tgaattccct ccccctgggc | |
| | cgggcccttc cggccgcccc cgcccgtgcc ccgctcgctc tcgggagatg | |
| | tttatttggg ctgtggcgtg aggagcgggc gggccagcgc cgcggagttt | |
| | cgggtccgag gagcctcgcg cggcgctgga gagagacaag atgtccgcca | |
| | gagctgcggc cgccaagagc acagcaatgg 481 aggaaacagc | |
| | tatatgggaa caacatacag tgacgcttca cagggctcct ggatttggat | |
| | ttggaattgc aatatctggt ggacgagata atcctcattt tcagagtggg | |
| | gaaacgtcaa tagtgatttc agatgtgctg aaaggaggac cagctgaagg | |
| | acagctacag gaaaatgacc gagttgcaat ggttaacgga gtttcaatgg | |
| | ataatgttga acatgctttt gctgttcagc aactaaggaa aagtgggaaa | |
| | aatgcaaaaa ttacaattag aaggaagaag aaagttcaaa taccagtaag | |
| | tcgtcctgat cctgaaccag tatctgataa tgaagaagat agttatgatg | |
| | aggaaataca tgatccaaga agtggccgga gtggtgtggt taacagaagg | |
| | agtgagaaga tttggccgag ggatagaagt gcaagtagag agaggagctt | |
| | gtccccgcgg tcagacaggc ggtcagtggc ttccagccag cctgctaaac | |
| | ctactaaagt cacactggtg aaatcccgga aaaatgaaga atatggtctt | |
| | cgattggcaa gccatatatt tgttaaggaa atttcacaag atagtttggc | |
| | agcaagagat ggcaatattc aagaaggtga tgttgtattg aagataaatg | |
| | gtactgtgac agaaaatatg tcattgacag atgcaaagac attgatagaa | |
| | aggtctaaag gcaaattaaa aatggtagtt caaagagatg aacgggctac | |
| | gctattgaat gtccctgatc tttctgacag catccactct gctaatgcct | |
| | ctgagagaga cgacatttca gaaattcagt cactggcatc agatcattct | |
| | ggtcgatcac acgataggcc tccccgccgc agccggtcac gatctcctga | |
| | ccagcggtca gagccttctg atcattccag gcactcgccg cagcagccaa | |
| | gcaatggcag tctccggagt agagatgaag agagaatttc taaacctggg | |
| | gctgtctcaa ctcctgtaaa gcatgctgat gatcacacac ctaaaacagt | |
| | ggaagaagtt acagttgaaa gaaatgagaa acaaacacct tctcttccag | |
| | aaccaaagcc tgtgtatgcc caagttgggc aaccagatgt ggatttacct | |
| | gtcagtccat ctgatggtgt cctacctaat tcaactcatg aagatgggat | |
| | tcttcggccc agcatgaaat tggtaaaatt cagaaaagga gatagtgtgg | |
| | gtttgcggct ggctggtgga aatgatgttg gaatatttgt agctggcgtt | |
| | ctagaagata gccctgcagc caaggaaggc ttagaggaag gtgatcaaat | |
| | tctcagggta aacaacgtag attttacaaa tatcataaga gaagaagccg | |
| | tccttttcct gcttgacctc cctaaaggag aagaagtgac catattggct | |
| | cagaagaaga aggatgttta tcgtcgcatt gtagaatcag atgtaggaga | |
| | ttctttctat attagaaccc attttgaata tgaaaaggaa tctccctatg gacttagttt | |
| | taacaaagga gaggtgttcc gtgttgtgga taccttgtac aatggaaaac | |
| | tgggctcttg gcttgctatt cgaattggta aaaatcataa ggaggtagaa | |
| | cgaggcatca tccctaataa gaacagagct gagcagctag ccagtgtaca | |
| | gtatacactt ccaaaaacag caggcggaga ccgtgctgac ttctggagat | |
| | tcagaggtct tcgcagctcc aagagaaatc ttcgaaaaag cagagaggat | |
| | ttgtccgctc agcctgttca aacaaagttt ccagcttatg aaagagtggt | |
| | tcttcgagaa gctggatttc tgaggcctgt aaccattttt ggaccaatag ctgatgttgc | |
| | cagagaaaag ctggcaagag aagaaccaga tatttatcaa attgcaaaga | |
| | gtgaaccacg agacgctgga actgaccaac gtagctctgg cattattcgc | |
| | ctgcatacaa taaagcaaat catagatcaa gacaaacatg ctttattaga | |
| | tgtaacacca aatgcagttg atcgtcttaa ctatgcccag tggtatccaa ttgttgtatt | |
| | tcttaaccct gattctaagc aaggagtaaa aacaatgaga atgaggttat | |
| | gtccagaatc tcggaaaagt gccaggaagt tatacgagcg atctcataaa | |
| | cttcgtaaaa ataatcacca tctttttaca actacaatta acttaaattc aatgaatgat | |
| | ggttggtatg gtgcgctgaa agaagcaatt caacaacagc aaaaccagct | |
| | ggtatgggtt tccgagggaa aggcggatgg tgctacaagt gatgaccttg | |
| | atttgcatga tgatcgtctg tcctacctgt cagctccagg tagtgaatac tcaatgtata | |
| | gcacggacag tagacacact tctgactatg aagacacaga cacagaaggc | |
| | ggggcctaca ctgatcaaga actagatgaa actcttaatg atgaggttgg | |
| | gactccaccg gagtctgcca ttacacggtc ctctgagcct gtaagagagg | |
| | actcctctgg aatgcatcat gaaaaccaaa catatcctcc ttactcacca | |
| | caagcgcagc cacaaccaat tcatagaata gactcccctg gatttaagcc | |
| | agcctctcaa cagaaagcag aagcttcatc tccagtccct tacctttcgc | |
| | ctgaaacaaa cccagcatca tcaacctctg ctgttaatca taatgtaaat | |
| | ttaactaatg tcagactgga ggagcccacc ccagctcctt ccacctctta | |
| | ctcaccacaa gctgattctt taagaacacc aagtactgag gcagctcaca | |
| | taatgctaag agatcaagaa ccatcattgt cgtcgcatgt agatccaaca | |
| | aaggtgtata gaaaggatcc atatcccgag gaaatgatga ggcagaacca | |
| | tgttttgaaa cagccagccg ttagtcaccc agggcacagg ccagacaaag | |
| | agcctaatct gacctatgaa ccccaactcc catacgtaga gaaacaagcc | |
| | agcagagacc tcgagcagcc cacatacaga tacgagtcct caagctatac | |
| | ggaccagttt tctcgaaact atgaacatcg tctgcgatac gaagatcgcg | |
| | tccccatgta tgaagaacag tggtcatatt atgatgacaa acagccctac | |
| | ccatctcggc caccttttga taatcagcac tctcaagacc ttgactccag | |
| | acagcatccc gaagagtcct cagaacgagg gtactttcca cgttttgaag | |
| | agccagcccc tctgtcttac gacagcagac cacgttacga acaggcacct | |
| | agagcatccg ccctgcggca cgaagagcag ccagctcctg ggtatgacac | |
| | acatggtaga ctcagaccgg aagcccagcc ccacccttca gcagggccca | |
| | agcctgcaga gtccaagcag tattttgagc aatattcacg cagttacgag | |
| | caagtaccac cccaaggatt tacctctaga gcaggtcatt ttgagcctct | |
| | ccatggtgct gcagctgtcc ctccgctgat accttcatct cagcataagc | |
| | cagaagctct gccttcaaac accaaaccac tgcctccacc cccaactcaa | |
| | accgaagaag aggaagatcc agcaatgaag ccacagtctg tactcaccag | |
| | agttaagatg tttgaaaaca aaagatctgc atccttagag accaagaagg | |
| | atgtaaatga cactggcagt tttaagcctc cagaagtagc atctaaacct | |
| | tcaggtgctc ccatcattgg tcccaaaccc acttctcaga atcaattcag | |
| | tgaacatgac aaaactctgt acaggatccc agaacctcaa aaacctcaac | |
| | tgaagccacc tgaagatatt gttcggtcca atcattatga ccctgaagaa | |
| | gatgaagaat attatcgaaa acagctgtca tactttgacc gaagaagttt | |
| | tgagaataag cctcctgcac acattgccgc cagccatctc tccgagcctg | |
| | caaagccagc gcattctcag aatcaatcaa atttttctag ttattcttca | |
| | aagggaaagc ctcctgaagc tgatggtgtg gatagatcat ttggcgagaa | |
| | acgctatgaa cccatccagg ccactccccc tcctcctcca ttgccctcgc | |
| | agtatgccca gccatctcag cctgtcacca gcgcgtctct ccacatacat | |
| | tctaagggag cacatggtga aggtaattca gtgtcattgg attttcagaa | |
| | ttccttagtg tccaaaccag acccacctcc atctcagaat aagccagcaa | |
| | ctttcagacc accaaaccga gaagatactg ctcaggcagc tttctatccc | |
| | cagaaaagtt ttccagataa agccccagtt aatggaactg aacagactca | |
| | gaaaacagtc actccagcat acaatcgatt cacaccaaaa ccatatacaa | |
| | gttctgcccg accatttgaa cgcaagtttg aaagtcctaa attcaatcac | |
| | aatcttctgc caagtgaaac tgcacataaa cctgacttgt cttcaaaaac | |
| | tcccacttct ccaaaaactc ttgtgaaatc gcacagtttg gcacagcctc | |
| | ctgagtttga cagtggagtt gaaactttct ctatccatgc agagaagcct | |
| | aaatatcaaa taaataatat cagcacagtg cctaaagcta ttcctgtgag | |
| | tccttcagct gtggaagagg atgaagatga agatggtcat actgtggtgg | |
| | ccacagcccg aggcatattt aacagcaatg ggggcgtgct gagttccata | |
| | gaaactggtg ttagtataat tatccctcaa ggagccattc ccgaaggagt | |
| | tgagcaggaa atctatttca aggtctgccg ggacaacagc atccttccac | |
| | ctttagataa agagaaaggt gaaacactgc tgagtccttt ggtgatgtgt | |
| | ggtccccatg gcctcaagtt cctgaagcct gtggagctgc gcttaccaca | |
| | ctgtgatcct aaaacctggc aaaacaagtg tcttcccgga gatccaaatt | |
| | atctcgttgg agcaaactgt gtttctgtcc ttattgacca cttttaactc ttgaaatata | |
| | ggaacttaaa taatgtgaaa ctggattaaa cttaatctaa atggaaccac | |
| | tctatcaagt attatacctt ttttagagtt gatactacag tttgttagta tgaggcattt | |
| | gtttgaactg ataaagatga gtgagcatgc ccctgaacca tggtcggaaa | |
| | acatgctaca cactgcatgt ttgtgattga cgggactgtt ggtattggct | |
| | agaggttcaa agatattttg ctttgtgatt tttgtaattt ttttatcgtc actgcttaac | |
| | ttcacatatt gatttccgtt aaaataccag ccagtaaatg ggggtgcatt tgaggtctgt | |
| | tctttccaaa gtacactgtt tcaaacttta ctatggccct ggcctagcat acgtacacat | |
| | tttattttat tatgcatgaa gtaatatgca cacatttttt aaatgcacct ggaatatata | |
| | accagtgttg tggatttaac agaaatgtac agcaaggaga tttacaactg | |
| | ggggagggtg aagtgaagac aatgacttac tgtacatgaa aacacatttt | |
| | tcttagggaa ggatacaaaa gcatgtgaga ctggttccat ggcctcttca | |
| | gatctctaac ttcaccatat taccacagac atactaacca gcagaaatgc | |
| | cttaccctca tgttcttaat tcttagctca ttctccttgt gttactaagt ttttatggct | |
| | tttgtgcatt atctagatac tgtatcatga caaagactga gtacgttgtg catttggtgg | |
| | tttcagaaat gtgttatcac ccagaagaaa atagtggtgt gatttgggga tatttttttc | |
| | ttttcttttc ttttcttttt tttttttttt gacaaggggc agtggtggtt ttctgttctt tctggctatg | |
| | catttgaaaa ttttgatgtt ttaaggatgc ttgtacataa tgcgtgcata ccacttttgt | |
| | tcttggtttg taaattaact tttataaact ttaccttttt tatacataaa caagaccacg | |
| | tttctaaagg ctacctttgt attctctcct gtacctcttg agccttgaac tttgacctct | |
| | gcagcaataa agcagcgttt ctatgacaca tgcaaggtca ttttttttaa | |
| | gaaaaaggat gcacagagtt gttacatttt taagtgctgc atttaaaaga | |
| | tacagttact cagaattctc tagtttgatt aaattcttgc aaagtatccc tactgtaatt | |
| | tgtgatacaa tgctgtgccc taaagtgtat ttttttacta atagacaatt tattatggca | |
| | catcagcacg atttctgttt agataataca ccactacatt ctgttaatca ttaggtgtga | |
| | ctgaatttct tttgccgtta ttaaaaatct caaatttcta aatctccaaa ataaaacttt | |
| | ttaaaataaa gtgctggctt ggtctgtttg cccactgttt tctagtttca tgcagcttta | |
| | taatcctgtt ttaaaatcct gcacacaaat ccctatcacc cagcgtcacc | |
| | taccacctcg tcgtctggtg ttgcatgcag aatttctccc cttggccagc | |
| | atgtacagat gggtgggcag tgctcatctg aagggctcag actgaagtgg | |
| | ggcagaagga cctggagaca gagtgggaga aggcagcagg ccgacttccc | |
| | cctgtgggta aacacacacc cctgcgtgga gaaacacccc tgcatgggga | |
| | cacacgtgcg tttgtgtgtg tgcgtgtaac catttgtata tggttttatt ccccagataa | |
| | atagcacggg cattgtttaa tgtcacccac atttggggga agaaaatggg | |
| | tttagtgagc ataaatattc cattgtggac atcctactta cttaactgcc acccctgttg | |
| | acatttggat tattgctata gtgtttcagt aaacaccttt gtggactgaa tccatctatg | |
| | tggattttat ttttttcatc tttgttaagg tataatttac atagagtaag ttactgtttt | |
| | taaaatgtat agttcaaatt ttgacaaaca catactgttt atttaccacg gttaatacat | |
| | acaacagttt ctttatcttc tctaatcatt tgtacccctt tatactcaac ccttcttcca | |
| | gcccctggca ggatgatctc ttctgtttct atagttgcct ttttgacagt gtcatataaa | |
| | tggaatcata caata | |

1. A method for treating a liver disease or regenerating a biliary system in a subject, wherein the method comprises administering of a pharmaceutically effective amount of a Tjp1 inhibitor to the subject.

2. (canceled)

3. The method of claim 1, wherein the Tjp1 inhibitor is a nucleic acid.

4. The method of claim 3, wherein the nucleic acid is selected from the group consisting of a short hairpin molecule, an shRNA, an siRNA, an antisense oligonucleotide (AON), a gapmer, and a short hairpin Antisense Oligonucleotide (shAON).

5. The method of claim 3, wherein the nucleic acid comprises at least 60% identity to a sequence selected from a group consisting of 5′-CGTGGATTGAACTTACTAAAT-3′ (SEQ ID NO: 4), 5′-ATTTAGTAAGTTCAATCCACG-3′ (SEQ ID NO: 91), 5′-CCGCGAAGTTATGAGCAAGTT-3′ (SEQ ID NO: 5), 5′-AACTTGCTCATAACTTCGCGG-3′ (SEQ ID NO: 92), 5′-CGGCCATTTGAACGCAAATTT-3′ (SEQ ID NO: 6), 5′-AAATTTGCGTTCAAATGGCCG-3′ (SEQ ID NO: 93), 5′-GCAATGGTTAACGGAGTTTCA-3′ (SEQ ID NO: 104), 5′-AATGGTTAACGGAGTTTCAAT-3′ (SEQ ID NO: 105), 5′-AAGGAAATTTCACAAGATAGT-3′ (SEQ ID NO: 106), 5′-TACAAGTGATGACCTTGATTT-3′ (SEQ ID NO: 107), 5′-ACTGATCAAGAACTAGATGAA-3′ (SEQ ID NO: 108), 5′-AAGAACTAGATGAAACTCTTA-3′ (SEQ ID NO: 109), 5′-CCCACCTTTAGATAAAGAGAA-3′ (SEQ ID NO: 110), 5′-CAGCACGATTTCTGTTTAGAT-3′ (SEQ ID NO: 111), 5′-AGCACGATTTCTGTTTAGATA-3′ (SEQ ID NO: 112), and 5′-TAGATAATACACCACTACATT-3′ (SEQ ID NO: 113).

6. The method of claim 3, wherein the nucleic acid comprises at least 80% identity to a sequence selected from a group consisting of 5′-CGTGGATTGAACTTACTAAAT-3′ (SEQ ID NO: 4), 5′-ATTTAGTAAGTTCAATCCACG-3′ (SEQ ID NO: 91), 5′-CCGCGAAGTTATGAGCAAGTT-3′ (SEQ ID NO: 5), 5′-AACTTGCTCATAACTTCGCGG-3′ (SEQ ID NO: 92), 5′-CGGCCATTTGAACGCAAATTT-3′ (SEQ ID NO: 6), 5′-AAATTTGCGTTCAAATGGCCG-3′ (SEQ ID NO: 93), 5′-GCAATGGTTAACGGAGTTTCA-3′ (SEQ ID NO: 104), 5′-AATGGTTAACGGAGTTTCAAT-3′ (SEQ ID NO: 105), 5′-AAGGAAATTTCACAAGATAGT-3′ (SEQ ID NO: 106), 5′-TACAAGTGATGACCTTGATTT-3′ (SEQ ID NO: 107), 5′-ACTGATCAAGAACTAGATGAA-3′ (SEQ ID NO: 108), 5′-AAGAACTAGATGAAACTCTTA-3′ (SEQ ID NO: 109), 5′-CCCACCTTTAGATAAAGAGAA-3′ (SEQ ID NO: 110), 5′-CAGCACGATTTCTGTTTAGAT-3′ (SEQ ID NO: 111), 5′-AGCACGATTTCTGTTTAGATA-3′ (SEQ ID NO: 112), and 5′-TAGATAATACACCACTACATT-3′ (SEQ ID NO: 113).

7. The method of claim 3, wherein the nucleic acid comprises a sequence selected from a group consisting of 5′-CGTGGATTGAACTTACTAAAT-3′ (SEQ ID NO: 4), 5′-ATTTAGTAAGTTCAATCCACG-3′ (SEQ ID NO: 91), 5′-CCGCGAAGTTATGAGCAAGTT-3′ (SEQ ID NO: 5), 5′-AACTTGCTCATAACTTCGCGG-3′ (SEQ ID NO: 92), 5′-CGGCCATTTGAACGCAAATTT-3′ (SEQ ID NO: 6), 5′-AAATTTGCGTTCAAATGGCCG-3′ (SEQ ID NO: 93), 5′-GCAATGGTTAACGGAGTTTCA-3′ (SEQ ID NO: 104), 5′-AATGGTTAACGGAGTTTCAAT-3′ (SEQ ID NO: 105), 5′-AAGGAAATTTCACAAGATAGT-3′ (SEQ ID NO: 106), 5′-TACAAGTGATGACCTTGATTT-3′ (SEQ ID NO: 107), 5′-ACTGATCAAGAACTAGATGAA-3′ (SEQ ID NO: 108), 5′-AAGAACTAGATGAAACTCTTA-3′ (SEQ ID NO: 109), 5′-CCCACCTTTAGATAAAGAGAA-3′ (SEQ ID NO: 110), 5′-CAGCACGATTTCTGTTTAGAT-3′ (SEQ ID NO: 111), 5′-AGCACGATTTCTGTTTAGATA-3′ (SEQ ID NO: 112), and 5′-TAGATAATACACCACTACATT-3′ (SEQ ID NO: 113).

8. The method of claim 3, wherein the nucleic acid comprises at least 60% identity to a sequence selected form the group consisting of:
i) a combination of SEQ ID NO: 4 and SEQ ID NO: 91, wherein SEQ ID NO: 4 is flanked at the 5′end by a nucleotide sequence comprising 1 to 10 nucleotides, wherein a nucleotide sequence of 1 to 20 nucleotides connects the 3′end of SEQ ID NO: 4 with the 5′end of SEQ ID NO: 91, and wherein SEQ ID NO: 91 is flanked at the 3′end by a nucleotide sequence comprising 1 to 10 nucleotides;
ii) a combination of SEQ ID NO: 5 and SEQ ID NO: 92, wherein SEQ ID NO: 5 is flanked at the 5′end by a nucleotide sequence comprising 1 to 10 nucleotides, wherein a nucleotide sequence of 1 to 20 nucleotides connects the 3′end of SEQ ID NO: 5 with the 5′end of SEQ ID NO: 92, and wherein SEQ ID NO: 92 is flanked at the 3′end by a nucleotide sequence comprising 1 to 10 nucleotides; and
iii) a combination of SEQ ID NO: 6 and SEQ ID NO: 93, wherein SEQ ID NO: 6 is flanked at the 5′end by a nucleotide sequence comprising 1 to 10 nucleotides, wherein a nucleotide sequence of 1 to 20 nucleotides connects the 3′end of SEQ ID NO: 6 with the 5′end of SEQ ID NO: 93, and wherein SEQ ID NO: 93 is flanked at the 3′end by a nucleotide sequence comprising 1 to 10 nucleotides.

9. The method of claim 3, wherein the nucleic acid comprises at least 60% identity to a sequence selected from the group consisting of:
(SEQ ID NO: 1)
5′-CCGGCGTGGATTGAACTTACTAAATCTCGAGATTTAGTAAGTTCAAT
CCACGTTTTTG-3′,
(SEQ ID NO: 2)
5′-CCGGCCGCGAAGTTATGAGCAAGTTCTCGAGAACTTGCTCATAACTT
CGCGGTTTTTG-3′,
and
(SEQ ID NO: 3)
5′-CCGGCGGCCATTTGAACGCAAATTTCTCGAGAAATTTGCGTTCAAAT
GGCCGTTTTTG-3′.

10. The method of claim 3, wherein the nucleic acid comprises at least 80% identity to a sequence selected from the group consisting of:
(SEQ ID NO: 1)
5′-CCGGCGTGGATTGAACTTACTAAATCTCGAGATTTAGTAAGTTCAAT
CCACGTTTTTG-3′,
(SEQ ID NO: 2)
5′-CCGGCCGCGAAGTTATGAGCAAGTTCTCGAGAACTTGCTCATAACTT
CGCGGTTTTTG-3′,
and
(SEQ ID NO: 3)
5′-CCGGCGGCCATTTGAACGCAAATTTCTCGAGAAATTTGCGTTCAAAT
GGCCGTTTTTG-3′.

11. The method of claim 3, wherein the nucleic acid is an siRNA.

12. The method of claim 11, wherein the siRNA comprises at least 60% identity to a sequence selected from a group consisting of
(SEQ ID NO: 94)
5′-UGAAACUCCGUUAACCAUUGC-3′,
(SEQ ID NO: 95)
5′-AUUGAAACUCCGUUAACCAUU-3′,
(SEQ ID NO: 96)
5′-ACUAUCUUGUGAAAUUUCCUU-3′,
(SEQ ID NO: 97)
5′-AAAUCAAGGUCAUCACUUGUA-3′,
(SEQ ID NO: 98)
5′-UUCAUCUAGUUCUUGAUCAGU-3′,
(SEQ ID NO: 99)
5′-UAAGAGUUUCAUCUAGUUCUU-3′,
(SEQ ID NO: 100)
5′-UUCUCUUUAUCUAAAGGUGGG-3′,
(SEQ ID NO: 101)
5′-AUCUAAACAGAAAUCGUGCUG-3′,
(SEQ ID NO: 102)
5′-UAUCUAAACAGAAAUCGUGCU-3′,
and
(SEQ ID NO: 103)
5′-AAUGUAGUGGUGUAUUAUCUA-3′.

13. The method of claim 11, wherein the siRNA comprises a sequence selected from a group consisting of
(SEQ ID NO: 94)
5′-UGAAACUCCGUUAACCAUUGC-3′,
(SEQ ID NO: 95)
5′-AUUGAAACUCCGUUAACCAUU-3′,
(SEQ ID NO: 96)
5′-ACUAUCUUGUGAAAUUUCCUU-3′,
(SEQ ID NO: 97)
5′-AAAUCAAGGUCAUCACUUGUA-3′,
(SEQ ID NO: 98)
5′-UUCAUCUAGUUCUUGAUCAGU-3′,
(SEQ ID NO: 99)
5′-UAAGAGUUUCAUCUAGUUCUU-3′,
(SEQ ID NO: 100)
5′-UUCUCUUUAUCUAAAGGUGGG-3′,
(SEQ ID NO: 101)
5′-AUCUAAACAGAAAUCGUGCUG-3′,
(SEQ ID NO: 102)
5′-UAUCUAAACAGAAAUCGUGCU-3′,
and
(SEQ ID NO: 103)
5′-AAUGUAGUGGUGUAUUAUCUA-3′.

14. The method of claim 1, wherein the liver disease is selected from the group consisting of cholestasis, liver cancer, alcoholic liver disease, nonalcoholic steatohepatitis (NASH), nonalcoholic fatty liver disease (NAFLD), cholestatic liver disease, hepatitis A, hepatitis B, hepatitis C, hepatitis D, hepatitis E, liver fibrosis, liver cirrhosis, cholestasis-related progressive bile duct injury, cystic fibrosis-associated liver disease, thioacetamide (TAA)-related liver disease, 3,5-Diethoxycarbonyl-1,4-Dihydrocollidine (DDC)-related liver disease, bile duct ligation liver injury, Yes-associated Protein (YAP)-related liver disease, Mdr2-related liver disease, a disease related to the exposure to medications that affect cholesterol/bile acid (BA) biosynthesis and/or metabolism, a disease related to genetic mutations that affect cholesterol/bile acid (BA) biosynthesis and/or metabolism, and a disease related to the compromise of the integrity the bile blood barrier.

15. The method of claim 14, wherein cholestasis is selected from a group consisting of intrahepatic cholestasis, primary biliary cholangitis (PBC), primary sclerosing cholangitis (PSC), pregnancy-related intrahepatic cholestasis, neonatal cholestasis, progressive familial intrahepatic cholestasis type 3, cholestatic fibrosis, and biliary atresia.

16. The method of claim 15, wherein the liver cancer is selected from a group consisting of hepatocellular carcinoma, cholangiocarcinoma, hepatoblastoma, and metastatic liver cancer.

17.-24. (canceled)


SEQUENCE LISTING (115 sequences)

SEQ ID NO: 1 - DNA, 58 bp, synthetic construct
  misc_binding 5..25: bound_moiety = Tjp1
  source 1..58: mol_type = other DNA
ccggcgtggattgaacttactaaatctcgagatttagtaagttcaatccacgtttttg

SEQ ID NO: 2 - DNA, 58 bp, synthetic construct
  misc_binding 5..25: bound_moiety = Tjp1
  source 1..58: mol_type = other DNA
ccggccgcgaagttatgagcaagttctcgagaacttgctcataacttcgcggtttttg

SEQ ID NO: 3 - DNA, 58 bp, synthetic construct
  source 1..58: mol_type = other DNA
  misc_binding 5..25: bound_moiety = Tjp1
ccggcggccatttgaacgcaaatttctcgagaaatttgcgttcaaatggccgtttttg

SEQ ID NO: 4 - DNA, 21 bp, synthetic construct
  source 1..21: mol_type = other DNA
cgtggattgaacttactaaat

SEQ ID NO: 5 - DNA, 21 bp, synthetic construct
  source 1..21: mol_type = other DNA
ccgcgaagttatgagcaagtt

SEQ ID NO: 6 - DNA, 21 bp, synthetic construct
  source 1..21: mol_type = other DNA
cggccatttgaacgcaaattt

SEQ ID NO: 7 - DNA, 8024 bp, Homo sapiens
  source 1..8024: mol_type = genomic DNA
  misc_feature 211..5796: note = Open reading frame
gccgccgggccacggcctgcaggggtggaaggggcggcggcggcgggagctgcgggccacgcacggcggcggcggcgggagcggccgggcacgcacggcgacggcggcggcgggagcgggagcccgggccacgcacggccgcagctgcgggtgcggcgggcgcgggagcggtgggagctgtggtggcggcggatggcccagagctgatctatgcggcgcctggaggggctgtgtctgcggcggcggcggaggcggcggggccctgcccgcgagcggagcggggacaagatgaagtaccagaaatacctgacggtgctgcagatggccatcggcgtcaccccctccaaccgcggcagcctcctgccgctcaagaggaagctgtgggtaacgccatcctctgaaaatcctaatggtgctacttctagtgtcagccaaggaaaaccctctttaagacgaattaaagggagattacacagaagcaaaagccttgatagcatggatttctgtgagctcactagcacagcaatggaggaaacagctatatgggaacaacatacagtgacgcttcacagggctcctggatttggatttggaattgcaatatctggtggacgagataatcctcattttcagagtggggaaacgtcaatagtgatttcagatgtgctgaaaggaggaccagctgaaggacagctacaggaaaatgaccgagttgcaatggttaacggagtttcaatggataatgttgaacatgcttttgctgttcagcaactaaggaaaagtgggaaaaatgcaaaaattacaattagaaggaagaagaaagttcaaataccagtaagtcgtcctgatcctgaaccagtatctgataatgaagaagatagttatgatgaggaaatacatgatccaagaagtggccggagtggtgtggttaacagaaggagtgagaagatttggccgagggatagaagtgcaagtagagagaggagcttgtccccgcggtcagacaggcggtcagtggcttccagccagcctgctaaacctactaaagtcacactggtgaaatcccggaaaaatgaagaatatggtcttcgattggcaagccatatatttgttaaggaaatttcacaagatagtttggcagcaagagatggcaatattcaagaaggtgatgttgtattgaagataaatggtactgtgacagaaaatatgtcattgacagatgcaaagacattgatagaaaggtctaaaggcaaattaaaaatggtagttcaaagagatgaacgggctacgctattgaatgtccctgatctttctgacagcatccactctgctaatgcctctgagagagacgacatttcagaaattcagtcactggcatcagatcattctggtcgatcacacgataggcctccccgccgcagccggtcacgatctcctgaccagcggtcagagccttctgatcattccaggcactcgccgcagcagccaagcaatggcagtctccggagtagagatgaagagagaatttctaaacctggggctgtctcaactcctgtaaagcatgctgatgatcacacacctaaaacagtggaagaagttacagttgaaagaaatgagaaacaaacaccttctcttccagaaccaaagcctgtgtatgcccaagttgggcaaccagatgtggatttacctgtcagtccatctgatggtgtcctacctaattcaactcatgaagatgggattcttcggcccagcatgaaattggtaaaattcagaaaaggagatagtgtgggtttgcggctggctggtggaaatgatgttggaatatttgtagctggcgttctagaagatagccctgcagccaaggaaggcttagaggaaggtgatcaaattctcagggtaaacaacgtagattttacaaatatcataagagaagaagccgtccttttcctgcttgacctccctaaaggagaagaagtgaccatattggctcagaagaagaaggatgtttatcgtcgcattgtagaatcagatgtaggagattctttctatattagaacccattttgaatatgaaaaggaatctccctatggacttagttttaacaaaggagaggtgttccgtgttgtggataccttgtacaatggaaaactgggctcttggcttgctattcgaattggtaaaaatcataaggaggtagaacgaggcatcatccctaataagaacagagctgagcagctagccagtgtacagtatacacttccaaaaacagcaggcggagaccgtgctgacttctggagattcagaggtcttcgcagctccaagagaaatcttcgaaaaagcagagaggatttgtccgctcagcctgttcaaacaaagtttccagcttatgaaagagtggttcttcgagaagctggatttctgaggcctgtaaccatttttggaccaatagctgatgttgccagagaaaagctggcaagagaagaaccagatatttatcaaattgcaaagagtgaaccacgagacgctggaactgaccaacgtagctctggcattattcgcctgcatacaataaagcaaatcatagatcaagacaaacatgctttattagatgtaacaccaaatgcagttgatcgtcttaactatgcccagtggtatccaattgttgtatttcttaaccctgattctaagcaaggagtaaaaacaatgagaatgaggttatgtccagaatctcggaaaagtgccaggaagttatacgagcgatctcataaacttcgtaaaaataatcaccatctttttacaactacaattaacttaaattcaatgaatgatggttggtatggtgcgctgaaagaagcaattcaacaacagcaaaaccagctggtatgggtttccgagggaaaggcggatggtgctacaagtgatgaccttgatttgcatgatgatcgtctgtcctacctgtcagctccaggtagtgaatactcaatgtatagcacggacagtagacacacttctgactatgaagacacagacacagaaggcggggcctacactgatcaagaactagatgaaactcttaatgatgaggttgggactccaccggagtctgccattacacggtcctctgagcctgtaagagaggactcctctggaatgcatcatgaaaaccaaacatatcctccttactcaccacaagcgcagccacaaccaattcatagaatagactcccctggatttaagccagcctctcaacagaaagcagaagcttcatctccagtcccttacctttcgcctgaaacaaacccagcatcatcaacctctgctgttaatcataatgtaaatttaactaatgtcagactggaggagcccaccccagctccttccacctcttactcaccacaagctgattctttaagaacaccaagtactgaggcagctcacataatgctaagagatcaagaaccatcattgtcgtcgcatgtagatccaacaaaggtgtatagaaaggatccatatcccgaggaaatgatgaggcagaaccatgttttgaaacagccagccgttagtcacccagggcacaggccagacaaagagcctaatctgacctatgaaccccaactcccatacgtagagaaacaagccagcagagacctcgagcagcccacatacagatacgagtcctcaagctatacggaccagttttctcgaaactatgaacatcgtctgcgatacgaagatcgcgtccccatgtatgaagaacagtggtcatattatgatgacaaacagccctacccatctcggccaccttttgataatcagcactctcaagaccttgactccagacagcatcccgaagagtcctcagaacgagggtactttccacgttttgaagagccagcccctctgtcttacgacagcagaccacgttacgaacaggcacctagagcatccgccctgcggcacgaagagcagccagctcctgggtatgacacacatggtagactcagaccggaagcccagccccacccttcagcagggcccaagcctgcagagtccaagcagtattttgagcaatattcacgcagttacgagcaagtaccaccccaaggatttacctctagagcaggtcattttgagcctctccatggtgctgcagctgtccctccgctgataccttcatctcagcataagccagaagctctgccttcaaacaccaaaccactgcctccacccccaactcaaaccgaagaagaggaagatccagcaatgaagccacagtctgtactcaccagagttaagatgtttgaaaacaaaagatctgcatccttagagaccaagaaggatgtaaatgacactggcagttttaagcctccagaagtagcatctaaaccttcaggtgctcccatcattggtcccaaacccacttctcagaatcaattcagtgaacatgacaaaactctgtacaggatcccagaacctcaaaaacctcaactgaagccacctgaagatattgttcggtccaatcattatgaccctgaagaagatgaagaatattatcgaaaacagctgtcatactttgaccgaagaagttttgagaataagcctcctgcacacattgccgccagccatctctccgagcctgcaaagccagcgcattctcagaatcaatcaaatttttctagttattcttcaaagggaaagcctcctgaagctgatggtgtggatagatcatttggcgagaaacgctatgaacccatccaggccactccccctcctcctccattgccctcgcagtatgcccagccatctcagcctgtcaccagcgcgtctctccacatacattctaagggagcacatggtgaaggtaattcagtgtcattggattttcagaattccttagtgtccaaaccagacccacctccatctcagaataagccagcaactttcagaccaccaaaccgagaagatactgctcaggcagctttctatccccagaaaagttttccagataaagccccagttaatggaactgaacagactcagaaaacagtcactccagcatacaatcgattcacaccaaaaccatatacaagttctgcccgaccatttgaacgcaagtttgaaagtcctaaattcaatcacaatcttctgccaagtgaaactgcacataaacctgacttgtcttcaaaaactcccacttctccaaaaactcttgtgaaatcgcacagtttggcacagcctcctgagtttgacagtggagttgaaactttctctatccatgcagagaagcctaaatatcaaataaataatatcagcacagtgcctaaagctattcctgtgagtccttcagctgtggaagaggatgaagatgaagatggtcatactgtggtggccacagcccgaggcatatttaacagcaatgggggcgtgctgagttccatagaaactggtgttagtataattatccctcaaggagccattcccgaaggagttgagcaggaaatctatttcaaggtctgccgggacaacagcatccttccacctttagataaagagaaaggtgaaacactgctgagtcctttggtgatgtgtggtccccatggcctcaagttcctgaagcctgtggagctgcgcttaccacactgtgcgtccatgactcctgacggttggtcttttgctctaaaatcatccgactcctcgtcgggtgatcctaaaacctggcaaaacaagtgtcttcccggagatccaaattatctcgttggagcaaactgtgtttctgtccttattgaccacttttaactcttgaaatataggaacttaaataatgtgaaactggattaaacttaatctaaatggaaccactctatcaagtattataccttttttagagttgatactacagtttgttagtatgaggcatttgtttgaactgataaagatgagtgagcatgcccctgaaccatggtcggaaaacatgctacacactgcatgtttgtgattgacgggactgttggtattggctagaggttcaaagatattttgctttgtgatttttgtaatttttttatcgtcactgcttaacttcacatattgatttccgttaaaataccagccagtaaatgggggtgcatttgaggtctgttctttccaaagtacactgtttcaaactttactatggccctggcctagcatacgtacacattttattttattatgcatgaagtaatatgcacacattttttaaatgcacctggaatatataaccagtgttgtggatttaacagaaatgtacagcaaggagatttacaactgggggagggtgaagtgaagacaatgacttactgtacatgaaaacacatttttcttagggaaggatacaaaagcatgtgagactggttccatggcctcttcagatctctaacttcaccatattaccacagacatactaaccagcagaaatgccttaccctcatgttcttaattcttagctcattctccttgtgttactaagtttttatggcttttgtgcattatctagatactgtatcatgacaaagactgagtacgttgtgcatttggtggtttcagaaatgtgttatcacccagaagaaaatagtggtgtgatttggggatatttttttcttttcttttcttttctttttttttttttttgacaaggggcagtggtggttttctgttctttctggctatgcatttgaaaattttgatgttttaaggatgcttgtacataatgcgtgcataccacttttgttcttggtttgtaaattaacttttataaactttaccttttttatacataaacaagaccacgtttctaaaggctacctttgtattctctcctgtacctcttgagccttgaactttgacctctgcagcaataaagcagcgtttctatgacacatgcaaggtcattttttttaagaaaaaggatgcacagagttgttacatttttaagtgctgcatttaaaagatacagttactcagaattctctagtttgattaaattcttgcaaagtatccctactgtaatttgtgatacaatgctgtgccctaaagtgtatttttttactaatagacaatttattatggcacatcagcacgatttctgtttagataatacaccactacattctgttaatcattaggtgtgactgaatttcttttgccgttattaaaaatctcaaatttctaaatctccaaaataaaactttttaaaataaagtgctggcttggtctgtttgcccactgttttctagtttcatgcagctttataatcctgttttaaaatcctgcacacaaatccctatcacccagcgtcacctaccacctcgtcgtctggtgttgcatgcagaatttctccccttggccagcatgtacagatgggtgggcagtgctcatctgaagggctcagactgaagtggggcagaaggacctggagacagagtgggagaaggcagcaggccgacttccccctgtgggtaaacacacacccctgcgtggagaaacacccctgcatggggacacacgtgcgtttgtgtgtgtgcgtgtaaccatttgtatatggttttattccccagataaatagcacgggcattgtttaatgtcacccacatttgggggaagaaaatgggtttagtgagcataaatattccattgtggacatcctacttacttaactgccacccctgttgacatttggattattgctatagtgtttcagtaaacacctttgtggactgaatccatctatgtggattttatttttttcatctttgttaaggtataatttacatagagtaagttactgtttttaaaatgtatagttcaaattttgacaaacacatactgtttatttaccacggttaatacatacaacagtttctttatcttctctaatcatttgtacccctttatactcaacccttcttccagcccctggcaggatgatctcttctgtttctatagttgcctttttgacagtgtcatataaatggaatcatacaata

SEQ ID NO: 8 - DNA, 5586 bp, Homo sapiens
  source 1..5586: mol_type = genomic DNA
atgcggcgcctggaggggctgtgtctgcggcggcggcggaggcggcggggccctgcccgcgagcggagcggggacaagatgaagtaccagaaatacctgacggtgctgcagatggccatcggcgtcaccccctccaaccgcggcagcctcctgccgctcaagaggaagctgtgggtaacgccatcctctgaaaatcctaatggtgctacttctagtgtcagccaaggaaaaccctctttaagacgaattaaagggagattacacagaagcaaaagccttgatagcatggatttctgtgagctcactagcacagcaatggaggaaacagctatatgggaacaacatacagtgacgcttcacagggctcctggatttggatttggaattgcaatatctggtggacgagataatcctcattttcagagtggggaaacgtcaatagtgatttcagatgtgctgaaaggaggaccagctgaaggacagctacaggaaaatgaccgagttgcaatggttaacggagtttcaatggataatgttgaacatgcttttgctgttcagcaactaaggaaaagtgggaaaaatgcaaaaattacaattagaaggaagaagaaagttcaaataccagtaagtcgtcctgatcctgaaccagtatctgataatgaagaagatagttatgatgaggaaatacatgatccaagaagtggccggagtggtgtggttaacagaaggagtgagaagatttggccgagggatagaagtgcaagtagagagaggagcttgtccccgcggtcagacaggcggtcagtggcttccagccagcctgctaaacctactaaagtcacactggtgaaatcccggaaaaatgaagaatatggtcttcgattggcaagccatatatttgttaaggaaatttcacaagatagtttggcagcaagagatggcaatattcaagaaggtgatgttgtattgaagataaatggtactgtgacagaaaatatgtcattgacagatgcaaagacattgatagaaaggtctaaaggcaaattaaaaatggtagttcaaagagatgaacgggctacgctattgaatgtccctgatctttctgacagcatccactctgctaatgcctctgagagagacgacatttcagaaattcagtcactggcatcagatcattctggtcgatcacacgataggcctccccgccgcagccggtcacgatctcctgaccagcggtcagagccttctgatcattccaggcactcgccgcagcagccaagcaatggcagtctccggagtagagatgaagagagaatttctaaacctggggctgtctcaactcctgtaaagcatgctgatgatcacacacctaaaacagtggaagaagttacagttgaaagaaatgagaaacaaacaccttctcttccagaaccaaagcctgtgtatgcccaagttgggcaaccagatgtggatttacctgtcagtccatctgatggtgtcctacctaattcaactcatgaagatgggattcttcggcccagcatgaaattggtaaaattcagaaaaggagatagtgtgggtttgcggctggctggtggaaatgatgttggaatatttgtagctggcgttctagaagatagccctgcagccaaggaaggcttagaggaaggtgatcaaattctcagggtaaacaacgtagattttacaaatatcataagagaagaagccgtccttttcctgcttgacctccctaaaggagaagaagtgaccatattggctcagaagaagaaggatgtttatcgtcgcattgtagaatcagatgtaggagattctttctatattagaacccattttgaatatgaaaaggaatctccctatggacttagttttaacaaaggagaggtgttccgtgttgtggataccttgtacaatggaaaactgggctcttggcttgctattcgaattggtaaaaatcataaggaggtagaacgaggcatcatccctaataagaacagagctgagcagctagccagtgtacagtatacacttccaaaaacagcaggcggagaccgtgctgacttctggagattcagaggtcttcgcagctccaagagaaatcttcgaaaaagcagagaggatttgtccgctcagcctgttcaaacaaagtttccagcttatgaaagagtggttcttcgagaagctggatttctgaggcctgtaaccatttttggaccaatagctgatgttgccagagaaaagctggcaagagaagaaccagatatttatcaaattgcaaagagtgaaccacgagacgctggaactgaccaacgtagctctggcattattcgcctgcatacaataaagcaaatcatagatcaagacaaacatgctttattagatgtaacaccaaatgcagttgatcgtcttaactatgcccagtggtatccaattgttgtatttcttaaccctgattctaagcaaggagtaaaaacaatgagaatgaggttatgtccagaatctcggaaaagtgccaggaagttatacgagcgatctcataaacttcgtaaaaataatcaccatctttttacaactacaattaacttaaattcaatgaatgatggttggtatggtgcgctgaaagaagcaattcaacaacagcaaaaccagctggtatgggtttccgagggaaaggcggatggtgctacaagtgatgaccttgatttgcatgatgatcgtctgtcctacctgtcagctccaggtagtgaatactcaatgtatagcacggacagtagacacacttctgactatgaagacacagacacagaaggcggggcctacactgatcaagaactagatgaaactcttaatgatgaggttgggactccaccggagtctgccattacacggtcctctgagcctgtaagagaggactcctctggaatgcatcatgaaaaccaaacatatcctccttactcaccacaagcgcagccacaaccaattcatagaatagactcccctggatttaagccagcctctcaacagaaagcagaagcttcatctccagtcccttacctttcgcctgaaacaaacccagcatcatcaacctctgctgttaatcataatgtaaatttaactaatgtcagactggaggagcccaccccagctccttccacctcttactcaccacaagctgattctttaagaacaccaagtactgaggcagctcacataatgctaagagatcaagaaccatcattgtcgtcgcatgtagatccaacaaaggtgtatagaaaggatccatatcccgaggaaatgatgaggcagaaccatgttttgaaacagccagccgttagtcacccagggcacaggccagacaaagagcctaatctgacctatgaaccccaactcccatacgtagagaaacaagccagcagagacctcgagcagcccacatacagatacgagtcctcaagctatacggaccagttttctcgaaactatgaacatcgtctgcgatacgaagatcgcgtccccatgtatgaagaacagtggtcatattatgatgacaaacagccctacccatctcggccaccttttgataatcagcactctcaagaccttgactccagacagcatcccgaagagtcctcagaacgagggtactttccacgttttgaagagccagcccctctgtcttacgacagcagaccacgttacgaacaggcacctagagcatccgccctgcggcacgaagagcagccagctcctgggtatgacacacatggtagactcagaccggaagcccagccccacccttcagcagggcccaagcctgcagagtccaagcagtattttgagcaatattcacgcagttacgagcaagtaccaccccaaggatttacctctagagcaggtcattttgagcctctccatggtgctgcagctgtccctccgctgataccttcatctcagcataagccagaagctctgccttcaaacaccaaaccactgcctccacccccaactcaaaccgaagaagaggaagatccagcaatgaagccacagtctgtactcaccagagttaagatgtttgaaaacaaaagatctgcatccttagagaccaagaaggatgtaaatgacactggcagttttaagcctccagaagtagcatctaaaccttcaggtgctcccatcattggtcccaaacccacttctcagaatcaattcagtgaacatgacaaaactctgtacaggatcccagaacctcaaaaacctcaactgaagccacctgaagatattgttcggtccaatcattatgaccctgaagaagatgaagaatattatcgaaaacagctgtcatactttgaccgaagaagttttgagaataagcctcctgcacacattgccgccagccatctctccgagcctgcaaagccagcgcattctcagaatcaatcaaatttttctagttattcttcaaagggaaagcctcctgaagctgatggtgtggatagatcatttggcgagaaacgctatgaacccatccaggccactccccctcctcctccattgccctcgcagtatgcccagccatctcagcctgtcaccagcgcgtctctccacatacattctaagggagcacatggtgaaggtaattcagtgtcattggattttcagaattccttagtgtccaaaccagacccacctccatctcagaataagccagcaactttcagaccaccaaaccgagaagatactgctcaggcagctttctatccccagaaaagttttccagataaagccccagttaatggaactgaacagactcagaaaacagtcactccagcatacaatcgattcacaccaaaaccatatacaagttctgcccgaccatttgaacgcaagtttgaaagtcctaaattcaatcacaatcttctgccaagtgaaactgcacataaacctgacttgtcttcaaaaactcccacttctccaaaaactcttgtgaaatcgcacagtttggcacagcctcctgagtttgacagtggagttgaaactttctctatccatgcagagaagcctaaatatcaaataaataatatcagcacagtgcctaaagctattcctgtgagtccttcagctgtggaagaggatgaagatgaagatggtcatactgtggtggccacagcccgaggcatatttaacagcaatgggggcgtgctgagttccatagaaactggtgttagtataattatccctcaaggagccattcccgaaggagttgagcaggaaatctatttcaaggtctgccgggacaacagcatccttccacctttagataaagagaaaggtgaaacactgctgagtcctttggtgatgtgtggtccccatggcctcaagttcctgaagcctgtggagctgcgcttaccacactgtgcgtccatgactcctgacggttggtcttttgctctaaaatcatccgactcctcgtcgggtgatcctaaaacctggcaaaacaagtgtcttcccggagatccaaattatctcgttggagcaaactgtgtttctgtccttattgaccacttttaa

SEQ ID NO: 9 - protein, 1803 aa, Homo sapiens
MRRLEGLCLRRRRRRRGPARERSGDKMKYQKYLTVLQMAIGVTPSTAMEETAIWEQHTVTLHRAPGFGFGIAISGGRDNPHFQSGETSIVISDVLKGGPAEGQLQENDRVAMVNGVSMDNVEHAFAVQQLRKSGKNAKITIRRKKKVQIPVSRPDPEPVSDNEEDSYDEEIHDPRSGRSGVVNRRSEKIWPRDRSASRERSLSPRSDRRSVASSQPAKPTKVTLVKSRKNEEYGLRLASHIFVKEISQDSLAARDGNIQEGDVVLKINGTVTENMSLTDAKTLIERSKGKLKMVVQRDERATLLNVPDLSDSIHSANASERDDISEIQSLASDHSGRSHDRPPRRSRSRSPDQRSEPSDHSRHSPQQPSNGSLRSRDEERISKPGAVSTPVKHADDHTPKTVEEVTVERNEKQTPSLPEPKPVYAQVGQPDVDLPVSPSDGVLPNSTHEDGILRPSMKLVKFRKGDSVGLRLAGGNDVGIFVAGVLEDSPAAKEGLEEGDQILRVNNVDFTNIIREEAVLFLLDLPKGEEVTILAQKKKDVYRRIVESDVGDSFYIRTHFEYEKESPYGLSFNKGEVFRVVDTLYNGKLGSWLAIRIGKNHKEVERGIIPNKNRAEQLASVQYTLPKTAGGDRADFWRFRGLRSSKRNLRKSREDLSAQPVQTKFPAYERVVLREAGFLRPVTIFGPIADVAREKLAREEPDIYQIAKSEPRDAGTDQRSSGIIRLHTIKQIIDQDKHALLDVTPNAVDRLNYAQWYPIVVFLNPDSKQGVKTMRMRLCPESRKSARKLYERSHKLRKNNHHLFTTTINLNSMNDGWYGALKEAIQQQQNQLVWVSEGKADGATSDDLDLHDDRLSYLSAPGSEYSMYSTDSRHTSDYEDTDTEGGAYTDQELDETLNDEVGTPPESAITRSSEPVREDSSGMHHENQTYPPYSPQAQPQPIHRIDSPGFKPASQQKAEASSPVPYLSPETNPASSTSAVNHNVNLTNVRLEEPTPAPSTSYSPQADSLRTPSTEAAHIMLRDQEPSLSSHVDPTKVYRKDPYPEEMMRQNHVLKQPAVSHPGHRPDKEPNLTYEPQLPYVEKQASRDLEQPTYRYESSSYTDQFSRNYEHRLRYEDRVPMYEEQWSYYDDKQPYPSRPPFDNQHSQDLDSRQHPEESSERGYFPRFEEPAPLSYDSRPRYEQAPRASALRHEEQPAPGYDTHGRLRPEAQPHPSAGPKPAESKQYFEQYSRSYEQVPPQGFTSRAGHFEPLHGAAAVPPLIPSSQHKPEALPSNTKPLPPPPTQTEEEEDPAMKPQSVLTRVKMFENKRSASLETKKDVNDTGSFKPPEVASKPSGAPIIGPKPTSQNQFSEHDKTLYRIPEPQKPQLKPPEDIVRSNHYDPEEDEEYYRKQLSYFDRRSFENKPPAHIAASHLSEPAKPAHSQNQSNFSSYSSKGKPPEADGVDRSFGEKRYEPIQATPPPPPLPSQYAQPSQPVTSASLHIHSKGAHGEGNSVSLDFQNSLVSKPDPPPSQNKPATFRPPNREDTAQAAFYPQKSFPDKAPVNGTEQTQKTVTPAYNRFTPKPYTSSARPFERKFESPKFNHNLLPSETAHKPDLSSKTPTSPKTLVKSHSLAQPPEFDSGVETFSIHAEKPKYQINNISTVPKAIPVSPSAVEEDEDEDGHTVVATARGIFNSNGGVLSSIETGVSIIIPQGAIPEGVEQEIYFKVCRDNSILPPLDKEKGETLLSPLVMCGPHGLKFLKPVELRLPHCASMTPDGWSFALKSSDSSSGDPKTWQNKCLPGDPNYLVGANCVSVLIDHF

SEQ ID NO: 10 - DNA, 7046 bp, Mus musculus
  source 1..7046: mol_type = genomic DNA
  misc_feature 404..5641: note = Open reading frame
cgcctgagttgcccgcgacggctctgcccgccgacggcacgtctctcggcggccgcgcgttccggggaagttacgtgcgggagcaggctttggaggagacgcccgagggtgtaggggacagccggaggcccgggtactgcggagcggcgagccggcggagggcggcggaggccgagcaggcggccgggtgtgccccgcggagaagcccgggcggggcggacgcttcccggacttttgtcccacttgaatcccctccccgtgggccgggcctttccggcctcccccgcccctgccccgctcgtccccgggagatgtttatgcggacggtggcgtgaggagcgggcgggccggcggcgcggagtttcgggtccgaggagcttcgcgcggcgcggagagagacaagatgtccgccagggccgcggccgctaagagcacagcaatggaggaaacagctatatgggaacagcacacagtgacgcttcacagggctcctgggtttggatttggaattgcaatctctggtggaagagataatcctcattttcagagtggggaaacctccatagtgatttctgatgtgttaaaaggagggccagctgaaggacagctacaggaaaatgaccgagttgcaatggttaacggagtttcaatggataacgttgaacatgcttttgctgttcagcagctaaggaagagtgggaaaaacgcaaaaattactatccgaaggaagaagaaagttcagatccctgtaagtcacccagatcctgagccggtgtctgataatgaagacgatagttatgacgaagaagtgcatgacccaagagctggccgcggtgctttagcgaacagaaggagcgagaagagctgggcaagggataggagtgcaagcagggagaggagcctgtcccctcgctcggacaggcggtccgtggcctccagtcagcccgcaaagcccaccaaggtcacactggtgaagtctcggaaaaatgaagaatatggtcttcgaccggccagccacatatttgtaaaggaaatttcacaagatagtttggcagcaagagatggtgacattcaagaaggggatgttgtcttgaagataaatggtactgtgacagaaaatatgtcattgacagatgcaaaaacactgatagaaaggtctaaaggcaagttaaaaatggtagtgcaaagagatgagcgggctaccttactgaacgtccctgacctttcggatagtatccattctgctaatgcctcggaaagagatgacatttcagaaattcagtcactagcgtcagaccattcaggtcgctcgcatgacaggccaccccgccgcagccagtcacgatctcctgaccaacgttcagagccctccgatcattccacgcagtctccacagcagcccagcaatggcagtctccggagcagagaggaagagcgaatgtctaaacctggggccatctcaactcctgtaaaacatgtagacgatcatccacccaaagcagtggaagaagttacagttgagaaaaatgagaagcagacgcccactcttccagaaccgaaacctgtgtatgctcaagttggacaaccagatgtggatttacccgtcagcccttctgatggtgctctgcctaattcagctcatgaagacgggatacttaggcccagcatgaaactggtaaaattcagaaaaggagatagtgtgggtttgcgactagctggtggaaatgatgtcggaatatttgtagctggcgttctagaagatagccctgcagccaaagaaggcttagaggaaggtgatcaaattctcagggtgaacaatgtagatttcacaaatatcataagggaagaggccgtccttttcctccttgacctccctaaaggtgaagaagtgaccatactggctcagaagaagaaggacgtttatcgccgcattgtagaatcagatgtaggagattcattctatattagaacgcattttgaatatgaaaaagaatctccttacggacttagttttaacaaaggagaggtgttccgggtcgtggatactttatacaatggaaagctgggctcttggcttgccattcgaattggcaaaaatcataaggaggtagaacgaggcatcatcccaaataagaacagagctgaacagttagccagtgtacagtacacacttccaaagacagcgggtggtgatcgggcagacttctggaggtttcgaggtcttcgcagctccaagagaaatcttcgaaaaagcagagaggacttgtcagctcagccagttcaaactaagttcccagcttatgaaagggttgttcttcgagaagctggattcctaagacctgtaaccatctttggaccaatagctgatgttgccagagaaaagttggcaagagaggagccagatatctatcagattgcaaaaagtgaactacgagacgctgggactgaccatcgtagctctggcatcattcgccttcatacaataaagcaaatcatagatcaggataaacatgctttattagatgtaacgccaaatgcggttgatcgtcttaattatgcgcagtggtatccaattgttgtgttccttaaccctgactctaagcaaggtgtaaaaacaatgaggatgaggttgtgtccggagtctcggaaaagcgccaggaagctatatgaacgctctcataagcttcgtaagaacaatcaccatctcttcacaactacaattaacttaaactcaatgaatgatggttggtacggtgccctgaaagaagcgattcagcagcaacagaaccagctggtgtgggtctctgaggggaaggcggatggtgctacaagtgatgaccttgatttgcatgacgatcgtctgtcctacctgtcagccccaggtagtgagtactcaatgtatagcacggacagtagacacacttctgactatgaagacacagatacagaaggcggggcctacactgatcaagaactagatgaaactcttaatgatgaggtggggactcccccggagtctgccattacacggtcctctgagcctgtaagagaggattcctctggaatgcatcatgaaaaccagacataccctccttactcaccacaagcgcagccacaagctattcatagaatagactcccctggacttaagccagcctctcaacagaaagcagaagcctcatctccagtcccttacctttcgcctgaaacaaccccagcatcatcagcctctgcagttaatcataatgtcagtgtaactaatgtcagcctggaggagcctgccccagcccctcccacctcgcacgcatcacagcctggttgtttaggagcaccaagtgctgaggcagctcacgtaggtctcagaggtgaaggaccaccattgccgccgcatgcagacccagcaaaggtgtacaggaaggagccatattctgaagaaatgatgagacaaaaccatattttaaaacaaccagctcttggtcacccagggcagaggccagataaagagccaaatctagcctatgaaccccaacttccatatatagaaaaacaagccagcagagaccttgagcagccgtcatacaggtatgaggtctcaagctacacagaccagttttctcggaactatgaccatcgcctacggtttgaagatcgaatccctacctatgaagaccagtggtcatattatgatgacaaacagccctaccaacctcggccttttgagaatcagcatccccgagacctggactccagacaacatcccgaagaggcttcagaacgaggttatttccagcgttttgaagagccagcccctctgtcgtacgacagtagaacacgctatgagcagctgcctcgaacctctactctacgacatgaagagcagccagcccctgcatatgaggtgcacaacaggtacaggccagaggcacagccctattcttcaacaggccctaagtcatctgagcccaagcagtactttgaccagtacccgcgaagttatgagcaagttccaccaccaggctttacctccaaaacaggccattacgagcctctccatggtgctgcagttgtccctcctctgataccttcctctcaacaaaagccagaagtcctgcgctcggctaccaaaccacagcctccacccccaaccctaactgaggaggaggaggatccagcaatgaaaccacagtctgtgctcaccagagtcaaaatgtttgaaaacaaaagatctgcgtctttggagaacaagaaagatgtgaatgacactgccagcttcaagcctccggaagtagcatctaaacctccaggtgcttctcttgctggccctaaacctgtccctcagagtcagtttagtgagcacgacaaaacgctctacaggctcccagagcctcagaaacctcaagtgaagccacccgaagatattgttcgatcaaatcattacgaccctgaagaggatgaagaatattaccggaaacagctctcctactttgaccgaagaagttttgagagcaagccttctgcacatcttcctgctggccatcactcagagcctgctaagccagtccattctcagagccagcccaacttccctagttactcttcaaagggaaaacccgaaactgatgctgtggatagatcattcagtgagaaacgttatgatccagcccaggccacgcctcctcctcctccgttgccctcacagtacagccagccagctccacctctgtccagctcttctctccacatacattccaagggcgcccagggtgaaggcaactcagtatcattggattttcagaactcatatatgtccaaaccagacccacccccatctcagagtaaaccagcaactttcagaccaccaactcgagaagacccccctcagactttctatccgcagaaaagtttcccagacaaagctccagttaacggagctgagcagactcagaaaaccatcactccggtgtacaatcgattcacaccaaagccgtacacaagttctgcccggccatttgaacgcaaatttgaaagtccgaagttcaaccataatcttctgccaagtgaaactgtacataaacctgaattgtcttcaaaaactcccacttccccaaaaactcttatgaaagctcatagttcaacacagcctccagagtttggcagtggagttgaaactttctctgttcacacagataagcctaaatatcaaatgaataatatcagcaccatgcctaaagctgtccctgtgagtccttcagctgtggaagaagatgaagatgaggatggtcatactgtagtggctacagctcgtggcatttttaacagcaatggtggtgtgttgagttccatagaaaccggtgttagtataattatcccacaaggagccattcctgaaggaattgagcaagaaatctatttcaaagtctgcagagacaatagcattctcccacctttagataaagagaaaggtgaaactctgctgagccccctagtgatgtgtgggccccatggcctcaagttcctgaagcccgtggagctacgcttgccacactgtgaccctaaaacctggcaaaacaagtgtcttcctggagatccgaattaccttgttggagccaactgtgtttctgtcctgattgaccacttttaattcttagtgtataggaactggactaagcaatgtgaacgtggattgaacttactaaatctaaatggaaccactctaatgagtattatattttcttagaatttatactgcaattggtagtattaagcatttgttggaactgatgaaggttagcgagcatgcccctgagccacggtcagaaagcatgctacaagctatgtgttattgagtgaagaactgtcaggcattggctagaggttcaaagatatttttgctttgtaatgatttttgtacttttttatggtcactgcttaacttcacatactgatttccgttaaaaataccagccagtaaatgggggtgcatttgagttctgttctttccaaagtacactcaaagtttattatggccttggcctagcatacacattttattttattatacatgaggtaatgtgcacacattttttacaaatgcacctggaatatataaccagtatagtggatttaacagaaatgtacagcagggggatttataacttggggagggagggtcaaatgaagacaattacttattgtatatgaaaacacatttcttttagggaaggacaccaaagcatgtgagcccggttcatggcctctgaatctataaattaacatatcaccacagacatgtaaccagcaggaatgccttaccctagtgggtttaattcttccatcatttcgctgtgtattactaagtttttatgagttctgtgcatatctagatactgtaccatggaaaagactgagtagattgtggacttgatggtttcagaaatgtgcaaattacctggaagaaaataatggtgtgacttaaaatatttttttgacagggggagtggcagtcttctgttctttgggctgtgcatctgagatgtttgatgctttcaggacgcatgtacataatgcgtgcataccacttttgttcttggtttgtaaattaacttttataaactttacctttttatacataaacagaaccaagtttctaaggctacctttgtattctctcctgtacctcttgagccttgaactttgacctctgcagcaataaagcagcatttctacacatacaaggtcatttttctaagaaaaagaatgcacagagttgttacatttttaagtgctgcatttaaaagatacagttactctagtttgattaaattcttgcaaagtatcccttctgtaaaatttgtgatacaatactgtgccctaaagtgtatttttttactaatagacagtttattatggcacatcagcacgatttctgtttagataatacaccactacattctgttaataatcagtaggtgtgaaaggatttcttttgtggttattaaaaaaaaatctcaaatttctaaatctgcaagaataaaactttttaaaat

SEQ ID NO: 11 - DNA, 5238 bp, Mus musculus
  source 1..5238: mol_type = genomic DNA
atgtccgccagggccgcggccgctaagagcacagcaatggaggaaacagctatatgggaacagcacacagtgacgcttcacagggctcctgggtttggatttggaattgcaatctctggtggaagagataatcctcattttcagagtggggaaacctccatagtgatttctgatgtgttaaaaggagggccagctgaaggacagctacaggaaaatgaccgagttgcaatggttaacggagtttcaatggataacgttgaacatgcttttgctgttcagcagctaaggaagagtgggaaaaacgcaaaaattactatccgaaggaagaagaaagttcagatccctgtaagtcacccagatcctgagccggtgtctgataatgaagacgatagttatgacgaagaagtgcatgacccaagagctggccgcggtgctttagcgaacagaaggagcgagaagagctgggcaagggataggagtgcaagcagggagaggagcctgtcccctcgctcggacaggcggtccgtggcctccagtcagcccgcaaagcccaccaaggtcacactggtgaagtctcggaaaaatgaagaatatggtcttcgaccggccagccacatatttgtaaaggaaatttcacaagatagtttggcagcaagagatggtgacattcaagaaggggatgttgtcttgaagataaatggtactgtgacagaaaatatgtcattgacagatgcaaaaacactgatagaaaggtctaaaggcaagttaaaaatggtagtgcaaagagatgagcgggctaccttactgaacgtccctgacctttcggatagtatccattctgctaatgcctcggaaagagatgacatttcagaaattcagtcactagcgtcagaccattcaggtcgctcgcatgacaggccaccccgccgcagccagtcacgatctcctgaccaacgttcagagccctccgatcattccacgcagtctccacagcagcccagcaatggcagtctccggagcagagaggaagagcgaatgtctaaacctggggccatctcaactcctgtaaaacatgtagacgatcatccacccaaagcagtggaagaagttacagttgagaaaaatgagaagcagacgcccactcttccagaaccgaaacctgtgtatgctcaagttggacaaccagatgtggatttacccgtcagcccttctgatggtgctctgcctaattcagctcatgaagacgggatacttaggcccagcatgaaactggtaaaattcagaaaaggagatagtgtgggtttgcgactagctggtggaaatgatgtcggaatatttgtagctggcgttctagaagatagccctgcagccaaagaaggcttagaggaaggtgatcaaattctcagggtgaacaatgtagatttcacaaatatcataagggaagaggccgtccttttcctccttgacctccctaaaggtgaagaagtgaccatactggctcagaagaagaaggacgtttatcgccgcattgtagaatcagatgtaggagattcattctatattagaacgcattttgaatatgaaaaagaatctccttacggacttagttttaacaaaggagaggtgttccgggtcgtggatactttatacaatggaaagctgggctcttggcttgccattcgaattggcaaaaatcataaggaggtagaacgaggcatcatcccaaataagaacagagctgaacagttagccagtgtacagtacacacttccaaagacagcgggtggtgatcgggcagacttctggaggtttcgaggtcttcgcagctccaagagaaatcttcgaaaaagcagagaggacttgtcagctcagccagttcaaactaagttcccagcttatgaaagggttgttcttcgagaagctggattcctaagacctgtaaccatctttggaccaatagctgatgttgccagagaaaagttggcaagagaggagccagatatctatcagattgcaaaaagtgaactacgagacgctgggactgaccatcgtagctctggcatcattcgccttcatacaataaagcaaatcatagatcaggataaacatgctttattagatgtaacgccaaatgcggttgatcgtcttaattatgcgcagtggtatccaattgttgtgttccttaaccctgactctaagcaaggtgtaaaaacaatgaggatgaggttgtgtccggagtctcggaaaagcgccaggaagctatatgaacgctctcataagcttcgtaagaacaatcaccatctcttcacaactacaattaacttaaactcaatgaatgatggttggtacggtgccctgaaagaagcgattcagcagcaacagaaccagctggtgtgggtctctgaggggaaggcggatggtgctacaagtgatgaccttgatttgcatgacgatcgtctgtcctacctgtcagccccaggtagtgagtactcaatgtatagcacggacagtagacacacttctgactatgaagacacagatacagaaggcggggcctacactgatcaagaactagatgaaactcttaatgatgaggtggggactcccccggagtctgccattacacggtcctctgagcctgtaagagaggattcctctggaatgcatcatgaaaaccagacataccctccttactcaccacaagcgcagccacaagctattcatagaatagactcccctggacttaagccagcctctcaacagaaagcagaagcctcatctccagtcccttacctttcgcctgaaacaaccccagcatcatcagcctctgcagttaatcataatgtcagtgtaactaatgtcagcctggaggagcctgccccagcccctcccacctcgcacgcatcacagcctggttgtttaggagcaccaagtgctgaggcagctcacgtaggtctcagaggtgaaggaccaccattgccgccgcatgcagacccagcaaaggtgtacaggaaggagccatattctgaagaaatgatgagacaaaaccatattttaaaacaaccagctcttggtcacccagggcagaggccagataaagagccaaatctagcctatgaaccccaacttccatatatagaaaaacaagccagcagagaccttgagcagccgtcatacaggtatgaggtctcaagctacacagaccagttttctcggaactatgaccatcgcctacggtttgaagatcgaatccctacctatgaagaccagtggtcatattatgatgacaaacagccctaccaacctcggccttttgagaatcagcatccccgagacctggactccagacaacatcccgaagaggcttcagaacgaggttatttccagcgttttgaagagccagcccctctgtcgtacgacagtagaacacgctatgagcagctgcctcgaacctctactctacgacatgaagagcagccagcccctgcatatgaggtgcacaacaggtacaggccagaggcacagccctattcttcaacaggccctaagtcatctgagcccaagcagtactttgaccagtacccgcgaagttatgagcaagttccaccaccaggctttacctccaaaacaggccattacgagcctctccatggtgctgcagttgtccctcctctgataccttcctctcaacaaaagccagaagtcctgcgctcggctaccaaaccacagcctccacccccaaccctaactgaggaggaggaggatccagcaatgaaaccacagtctgtgctcaccagagtcaaaatgtttgaaaacaaaagatctgcgtctttggagaacaagaaagatgtgaatgacactgccagcttcaagcctccggaagtagcatctaaacctccaggtgcttctcttgctggccctaaacctgtccctcagagtcagtttagtgagcacgacaaaacgctctacaggctcccagagcctcagaaacctcaagtgaagccacccgaagatattgttcgatcaaatcattacgaccctgaagaggatgaagaatattaccggaaacagctctcctactttgaccgaagaagttttgagagcaagccttctgcacatcttcctgctggccatcactcagagcctgctaagccagtccattctcagagccagcccaacttccctagttactcttcaaagggaaaacccgaaactgatgctgtggatagatcattcagtgagaaacgttatgatccagcccaggccacgcctcctcctcctccgttgccctcacagtacagccagccagctccacctctgtccagctcttctctccacatacattccaagggcgcccagggtgaaggcaactcagtatcattggattttcagaactcatatatgtccaaaccagacccacccccatctcagagtaaaccagcaactttcagaccaccaactcgagaagacccccctcagactttctatccgcagaaaagtttcccagacaaagctccagttaacggagctgagcagactcagaaaaccatcactccggtgtacaatcgattcacaccaaagccgtacacaagttctgcccggccatttgaacgcaaatttgaaagtccgaagttcaaccataatcttctgccaagtgaaactgtacataaacctgaattgtcttcaaaaactcccacttccccaaaaactcttatgaaagctcatagttcaacacagcctccagagtttggcagtggagttgaaactttctctgttcacacagataagcctaaatatcaaatgaataatatcagcaccatgcctaaagctgtccctgtgagtccttcagctgtggaagaagatgaagatgaggatggtcatactgtagtggctacagctcgtggcatttttaacagcaatggtggtgtgttgagttccatagaaaccggtgttagtataattatcccacaaggagccattcctgaaggaattgagcaagaaatctatttcaaagtctgcagagacaatagcattctcccacctttagataaagagaaaggtgaaactctgctgagccccctagtgatgtgtgggccccatggcctcaagttcctgaagcccgtggagctacgcttgccacactgtgaccctaaaacctggcaaaacaagtgtcttcctggagatccgaattaccttgttggagccaactgtgtttctgtcctgattgaccacttttaa

SEQ ID NO: 12 - protein, 1745 aa, Mus musculus
MSARAAAAKSTAMEETAIWEQHTVTLHRAPGFGFGIAISGGRDNPHFQSGETSIVISDVLKGGPAEGQLQENDRVAMVNGVSMDNVEHAFAVQQLRKSGKNAKITIRRKKKVQIPVSHPDPEPVSDNEDDSYDEEVHDPRAGRGALANRRSEKSWARDRSASRERSLSPRSDRRSVASSQPAKPTKVTLVKSRKNEEYGLRPASHIFVKEISQDSLAARDGDIQEGDVVLKINGTVTENMSLTDAKTLIERSKGKLKMVVQRDERATLLNVPDLSDSIHSANASERDDISEIQSLASDHSGRSHDRPPRRSQSRSPDQRSEPSDHSTQSPQQPSNGSLRSREEERMSKPGAISTPVKHVDDHPPKAVEEVTVEKNEKQTPTLPEPKPVYAQVGQPDVDLPVSPSDGALPNSAHEDGILRPSMKLVKFRKGDSVGLRLAGGNDVGIFVAGVLEDSPAAKEGLEEGDQILRVNNVDFTNIIREEAVLFLLDLPKGEEVTILAQKKKDVYRRIVESDVGDSFYIRTHFEYEKESPYGLSFNKGEVFRVVDTLYNGKLGSWLAIRIGKNHKEVERGIIPNKNRAEQLASVQYTLPKTAGGDRADFWRFRGLRSSKRNLRKSREDLSAQPVQTKFPAYERVVLREAGFLRPVTIFGPIADVAREKLAREEPDIYQIAKSELRDAGTDHRSSGIIRLHTIKQIIDQDKHALLDVTPNAVDRLNYAQWYPIVVFLNPDSKQGVKTMRMRLCPESRKSARKLYERSHKLRKNNHHLFTTTINLNSMNDGWYGALKEAIQQQQNQLVWVSEGKADGATSDDLDLHDDRLSYLSAPGSEYSMYSTDSRHTSDYEDTDTEGGAYTDQELDETLNDEVGTPPESAITRSSEPVREDSSGMHHENQTYPPYSPQAQPQAIHRIDSPGLKPASQQKAEASSPVPYLSPETTPASSASAVNHNVSVTNVSLEEPAPAPPTSHASQPGCLGAPSAEAAHVGLRGEGPPLPPHADPAKVYRKEPYSEEMMRQNHILKQPALGHPGQRPDKEPNLAYEPQLPYIEKQASRDLEQPSYRYEVSSYTDQFSRNYDHRLRFEDRIPTYEDQWSYYDDKQPYQPRPFENQHPRDLDSRQHPEEASERGYFQRFEEPAPLSYDSRTRYEQLPRTSTLRHEEQPAPAYEVHNRYRPEAQPYSSTGPKSSEPKQYFDQYPRSYEQVPPPGFTSKTGHYEPLHGAAVVPPLIPSSQQKPEVLRSATKPQPPPPTLTEEEEDPAMKPQSVLTRVKMFENKRSASLENKKDVNDTASFKPPEVASKPPGASLAGPKPVPQSQFSEHDKTLYRLPEPQKPQVKPPEDIVRSNHYDPEEDEEYYRKQLSYFDRRSFESKPSAHLPAGHHSEPAKPVHSQSQPNFPSYSSKGKPETDAVDRSFSEKRYDPAQATPPPPPLPSQYSQPAPPLSSSSLHIHSKGAQGEGNSVSLDFQNSYMSKPDPPPSQSKPATFRPPTREDPPQTFYPQKSFPDKAPVNGAEQTQKTITPVYNRFTPKPYTSSARPFERKFESPKFNHNLLPSETVHKPELSSKTPTSPKTLMKAHSSTQPPEFGSGVETFSVHTDKPKYQMNNISTMPKAVPVSPSAVEEDEDEDGHTVVATARGIFNSNGGVLSSIETGVSIIIPQGAIPEGIEQEIYFKVCRDNSILPPLDKEKGETLLSPLVMCGPHGLKFLKPVELRLPHCDPKTWQNKCLPGDPNYLVGANCVSVLIDHF

SEQ ID NO: 13 - DNA, 24 bp, synthetic construct
  source 1..24: mol_type = other DNA
cttctctgaccctacacagctacc

SEQ ID NO: 14 - DNA, 20 bp, synthetic construct
  source 1..20: mol_type = other DNA
atcgtgtgggaaagacaagc

SEQ ID NO: 15 - DNA, 25 bp, synthetic construct
  source 1..25: mol_type = other DNA
ccatttgtcctcatctcttactaac

SEQ ID NO: 16 - DNA, 27 bp, synthetic construct
  source 1..27: mol_type = other DNA
gattgggcactgtcaattaatgggctt

SEQ ID NO: 17 - DNA, 23 bp, synthetic construct
  source 1..23: mol_type = other DNA
ggcccagagcaagagaggtatcc

SEQ ID NO: 18 - DNA, 21 bp, synthetic construct
  source 1..21: mol_type = other DNA
acgcacgatttccctctcagc

SEQ ID NO: 19 - DNA, 24 bp, synthetic construct
  source 1..24: mol_type = other DNA
agcacagcaatggaggaaacagct

SEQ ID NO: 20 - DNA, 24 bp, synthetic construct
  source 1..24: mol_type = other DNA
ctgggtgacttacagggatctgaa

SEQ ID NO: 21 - DNA, 19 bp, synthetic construct
  source 1..19: mol_type = other DNA
agcgtgatcagcggttttg

SEQ ID NO: 22 - DNA, 20 bp, synthetic construct
  source 1..20: mol_type = other DNA
cctggttctggcgctctatg

SEQ ID NO: 23 - DNA, 21 bp, synthetic construct
  source 1..21: mol_type = other DNA
gtcatttgctccaaactggcg

SEQ ID NO: 24 - DNA, 20 bp, synthetic construct
  source 1..20: mol_type = other DNA
agcccatcgttgttctggat

SEQ ID NO: 25 - DNA, 23 bp, synthetic construct
  source 1..23: mol_type = other DNA
gactggaagagcggagagtactg

SEQ ID NO: 26 - DNA, 23 bp, synthetic construct
  source 1..23: mol_type = other DNA
caggtctgacctgtctccatgtt

SEQ ID NO: 27 - DNA, 18 bp, synthetic construct
  source 1..18: mol_type = other DNA
gcagtggctgaaccaagg

SEQ ID NO: 28 - DNA, 18 bp, synthetic construct
  source 1..18: mol_type = other DNA
agcagtgagcgctgaatc

SEQ ID NO: 29 - DNA, 20 bp, synthetic construct
  source 1..20: mol_type = other DNA
ggcatctggcatcctcttgt

SEQ ID NO: 30 - DNA, 21 bp, synthetic construct
  source 1..21: mol_type = other DNA
tagcccttatgaccaggtccg

SEQ ID NO: 31 - DNA, 20 bp, synthetic construct
  source 1..20: mol_type = other DNA
gcggtcctcaccatgagtcc

SEQ ID NO: 32 - DNA, 23 bp, synthetic construct
  source 1..23: mol_type = other DNA
tagcggtacaagtatgcctctgc

SEQ ID NO: 33 - DNA, 21 bp, synthetic construct
  source 1..21: mol_type = other DNA
ccttcgtgactactgccgagc

SEQ ID NO: 34 - DNA, 20 bp, synthetic construct
  source 1..20: mol_type = other DNA
ttcgtggatgcccgctgact

SEQ ID NO: 35 - DNA, 20 bp, synthetic construct
  source 1..20: mol_type = other DNA
gcagaagaagccacgctatg

SEQ ID NO: 36 - DNA, 20 bp, synthetic construct
  source 1..20: mol_type = other DNA
caggtggagtcgtaaggcaa

SEQ ID NO: 37 - DNA, 19 bp, synthetic construct
  source 1..19: mol_type = other DNA
gagtcggcatgaatcgctg

SEQ ID NO: 38 - DNA, 20 bp, synthetic construct
  source 1..20: mol_type = other DNA
tcaaaggagcggatggagtg

SEQ ID NO: 39 - DNA, 24 bp, synthetic construct
  source 1..24: mol_type = other DNA
acattaagaagggcaaaaagtgca

SEQ ID NO: 40 - DNA, 24 bp, synthetic construct
  source 1..24: mol_type = other DNA
aacttagccctgtatgtcttcaca

SEQ ID NO: 41 - DNA, 19 bp, synthetic construct
  source 1..19: mol_type = other DNA
cacgtcgtagcaaaccacc

SEQ ID NO: 42 - DNA, 18 bp, synthetic construct
  source 1..18: mol_type = other DNA
ggtgaggagcacgtagtc

SEQ ID NO: 43 - DNA, 21 bp, synthetic construct
  source 1..21: mol_type = other DNA
cctctggtcttctggagtacc

SEQ ID NO: 44 - DNA, 20 bp, synthetic construct
  source 1..20: mol_type = other DNA
actccttctgtgactccagc

SEQ ID NO: 45 - DNA, 21 bp, synthetic construct
  source 1..21: mol_type = other DNA
tgccaccttttgacagtgatg

SEQ ID NO: 46 - DNA, 20 bp, synthetic construct
  source 1..20: mol_type = other DNA
atgtgctgctgcgagatttg

SEQ ID NO: 47 - DNA, 20 bp, synthetic construct
  source 1..20: mol_type = other DNA
aacatgcaacctgccacaac

SEQ ID NO: 48 - DNA, 20 bp, synthetic construct
  source 1..20: mol_type = other DNA
ttcacaggattcgtccaggc

SEQ ID NO: 49 - DNA, 21 bp, synthetic construct
  source 1..21: mol_type = other DNA
acactttcactccaatcgtcc

SEQ ID NO: 50 - DNA, 20 bp, synthetic construct
  source 1..20: mol_type = other DNA
tgccctttccgttgttgtcc

SEQ ID NO: 51 - DNA, 23 bp, synthetic construct
  source 1..23: mol_type = other DNA
gcagccagattggctcttatttt

SEQ ID NO: 52 - DNA, 17 bp, synthetic construct
  source 1..17: mol_type = other DNA
gccctcgaggcaaggga

SEQ ID NO: 53 - DNA, 22 bp, synthetic construct
  source 1..22: mol_type = other DNA
ggtggtgagagcttgaaaatga

SEQ ID NO: 54 - DNA, 21 bp, synthetic construct
  source 1..21: mol_type = other DNA
gtggttcttggaggtgccttt

SEQ ID NO: 55 - DNA, 20 bp, synthetic construct
  source 1..20: mol_type = other DNA
ctatggaagccctgcgtgac

SEQ ID NO: 56 - DNA, 20 bp, synthetic construct
  source 1..20: mol_type = other DNA
gagcacagcctcagaacctc

SEQ ID NO: 57 - DNA, 20 bp, synthetic construct
  source 1..20: mol_type = other DNA
ttgcaaatgctgcctcaacc

SEQ ID NO: 58 - DNA, 21 bp, synthetic construct
  source 1..21: mol_type = other DNA
taacagttcgcacacatggct

SEQ ID NO: 59 - DNA, 20 bp, synthetic construct
  source 1..20: mol_type = other DNA
gagacaggagggcaagtacc

SEQ ID NO: 60 - DNA, 20 bp, synthetic construct
  source 1..20: mol_type = other DNA
taaggcatccgtgtagagcg

SEQ ID NO: 61 - DNA, 20 bp, synthetic construct
  source 1..20: mol_type = other DNA
gcattaccaagaacgccgtg

SEQ ID NO: 62 - DNA, 24 bp, synthetic construct
  source 1..24: mol_type = other DNA
ctggatttcagttaacaaacctgt

SEQ ID NO: 63 - DNA, 21 bp, synthetic construct
  source 1..21: mol_type = other DNA
ccaggccagtagagtggtagc

SEQ ID NO: 64 - DNA, 19 bp, synthetic construct
  source 1..19: mol_type = other DNA
ggcaggcacgtacaaactc

SEQ ID NO: 65 - DNA, 24 bp, synthetic construct
  source 1..24: mol_type = other DNA
tacgtcctcaaggcaggcatgatc

SEQ ID NO: 66 - DNA, 24 bp, synthetic construct
  source 1..24: mol_type = other DNA
gtagccagtaagtgtggtgtcatg

SEQ ID NO: 67 - DNA, 21 bp, synthetic construct
  source 1..21: mol_type = other DNA
acgcaagatccaacagagtgt

SEQ ID NO: 68 - DNA, 20 bp, synthetic construct
  source 1..20: mol_type = other DNA
gggccaacaatcttccccat

SEQ ID NO: 69 - DNA, 22 bp, synthetic construct
  source 1..22: mol_type = other DNA
tgagttcaggacaccaagactg

SEQ ID NO: 70 - DNA, 19 bp, synthetic construct
  source 1..19: mol_type = other DNA
accgcctaccctatgcctt

SEQ ID NO: 71 - DNA, 22 bp, synthetic construct
  source 1..22: mol_type = other DNA
catctgtagggttgttgagtga

SEQ ID NO: 72 - DNA, 20 bp, synthetic construct
  source 1..20: mol_type = other DNA
caaagaagccaactcgagcg

SEQ ID NO: 73 - DNA, 20 bp, synthetic construct
  source 1..20: mol_type = other DNA
acggcgactttgaactaggc

SEQ ID NO: 74 - DNA, 22 bp, synthetic construct
  source 1..22: mol_type = other DNA
ccagtcagagtatcggaacagt

SEQ ID NO: 75 - DNA, 24 bp, synthetic construct
  source 1..24: mol_type = other DNA
gcaactctactttttggaatctct

SEQ ID NO: 76 - DNA, 20 bp, synthetic construct
  source 1..20: mol_type = other DNA
ccaagagccaaagaaagccc

SEQ ID NO: 77 - DNA, 20 bp, synthetic construct
  source 1..20: mol_type = other DNA
tgaggatgcggtcctactga

SEQ ID NO: 78 - DNA, 20 bp, synthetic construct
  source 1..20: mol_type = other DNA
ggccgtggggtcagaaataa

SEQ ID NO: 79 - DNA, 20 bp, synthetic construct
  source 1..20: mol_type = other DNA
gaccatgaagccttgcaaaa

SEQ ID NO: 80 - DNA, 20 bp, synthetic construct
  source 1..20: mol_type = other DNA
ttgggcatagtccggttctg

SEQ ID NO: 81 - DNA, 19 bp, synthetic construct
  source 1..19: mol_type = other DNA
acctgggtgctcctagcaa

SEQ ID NO: 82 - DNA, 19 bp, synthetic construct
  source 1..19: mol_type = other DNA
aaatggcagaggtttgggc

SEQ ID NO: 83 - DNA, 21 bp, synthetic construct
  source 1..21: mol_type = other DNA
cagacccggagttacccaaag

SEQ ID NO: 84 - DNA, 20 bp, synthetic construct
  source 1..20: mol_type = other DNA
aaaccgactttggagccgag

SEQ ID NO: 85 - DNA, 20 bp, synthetic construct
  source 1..20: mol_type = other DNA
ggcctgaccttggctcatag

SEQ ID NO: 86 - DNA, 22 bp, synthetic construct
  source 1..22: mol_type = other DNA
acatctgtgtgtcctagactgt

SEQ ID NO: 87 - DNA, 19 bp, synthetic construct
  source 1..19: mol_type = other DNA
tgtgtgtgttcggtccctc

SEQ ID NO: 88 - DNA, 20 bp, synthetic construct
  source 1..20: mol_type = other DNA
gcttcaggtgctatgaccac

SEQ ID NO: 89 - DNA, 25 bp, synthetic construct
  source 1..25: mol_type = other DNA
cctcaaaggaaatgttctattcgga

SEQ ID NO: 90 - DNA, 20 bp, synthetic construct
  source 1..20: mol_type = other DNA
tccagctcatctggccctaa

SEQ ID NO: 91 - DNA, 21 bp, synthetic construct
  source 1..21: mol_type = other DNA
atttagtaagttcaatccacg

SEQ ID NO: 92 - DNA, 21 bp, synthetic construct
  source 1..21: mol_type = other DNA
aacttgctcataacttcgcgg

SEQ ID NO: 93 - DNA, 21 bp, synthetic construct
  source 1..21: mol_type = other DNA
aaatttgcgttcaaatggccg

SEQ ID NO: 94 - RNA, 21 nt, synthetic construct
  source 1..21: mol_type = other RNA
tgaaactccgttaaccattgc

SEQ ID NO: 95 - RNA, 21 nt, synthetic construct
  source 1..21: mol_type = other RNA
attgaaactccgttaaccatt

SEQ ID NO: 96 - RNA, 21 nt, synthetic construct
  source 1..21: mol_type = other RNA
actatcttgtgaaatttcctt

SEQ ID NO: 97 - RNA, 21 nt, synthetic construct
  source 1..21: mol_type = other RNA
aaatcaaggtcatcacttgta

SEQ ID NO: 98 - RNA, 21 nt, synthetic construct
  source 1..21: mol_type = other RNA
ttcatctagttcttgatcagt

SEQ ID NO: 99 - RNA, 21 nt, synthetic construct
  source 1..21: mol_type = other RNA
taagagtttcatctagttctt

SEQ ID NO: 100 - RNA, 21 nt, synthetic construct
  source 1..21: mol_type = other RNA
ttctctttatctaaaggtggg

SEQ ID NO: 101 - RNA, 21 nt, synthetic construct
  source 1..21: mol_type = other RNA
atctaaacagaaatcgtgctg

SEQ ID NO: 102 - RNA, 21 nt, synthetic construct
  source 1..21: mol_type = other RNA
tatctaaacagaaatcgtgct

SEQ ID NO: 103 - RNA, 21 nt, synthetic construct
  source 1..21: mol_type = other RNA
aatgtagtggtgtattatcta

SEQ ID NO: 104 - DNA, 21 bp, synthetic construct
  source 1..21: mol_type = other DNA
gcaatggttaacggagtttca

SEQ ID NO: 105 - DNA, 21 bp, synthetic construct
  source 1..21: mol_type = other DNA
aatggttaacggagtttcaat

SEQ ID NO: 106 - DNA, 21 bp, synthetic construct
  source 1..21: mol_type = other DNA
aaggaaatttcacaagatagt

SEQ ID NO: 107 - DNA, 21 bp, synthetic construct
  source 1..21: mol_type = other DNA
tacaagtgatgaccttgattt

SEQ ID NO: 108 - DNA, 21 bp, synthetic construct
  source 1..21: mol_type = other DNA
actgatcaagaactagatgaa

SEQ ID NO: 109 - DNA, 21 bp, synthetic construct
  source 1..21: mol_type = other DNA
aagaactagatgaaactctta

SEQ ID NO: 110 - DNA, 21 bp, synthetic construct
  source 1..21: mol_type = other DNA
cccacctttagataaagagaa

SEQ ID NO: 111 - DNA, 21 bp, synthetic construct
  source 1..21: mol_type = other DNA
cagcacgatttctgtttagat

SEQ ID NO: 112 - DNA, 21 bp, synthetic construct
  source 1..21: mol_type = other DNA
agcacgatttctgtttagata

SEQ ID NO: 113 - DNA, 21 bp, synthetic construct
  source 1..21: mol_type = other DNA
tagataatacaccactacatt

SEQ ID NO: 114 - DNA, 7054 bp, Mus musculus
  source 1..7054: mol_type = genomic DNA
gtggccgcctgagttgcccgcgacggctctgcccgccgacggcacgtctctcggcggccgcgcgttccggggaagttacgtgcgggagcaggctttggaggagacgcccgagggtgtaggggacagccggaggcccgggtactgcggagcggcgagccggcggagggcggcggaggccgagcaggcggccgggtgtgccccgcggagaagcccgggcggggcggacgcttcccggacttttgtcccacttgaatcccctccccgtgggccgggcctttccggcctcccccgcccctgccccgctcgtccccgggagatgtttatgcggacggtggcgtgaggagcgggcgggccggcggcgcggagtttcgggtccgaggagcttcgcgcggcgcggagagagacaagatgtccgccagggccgcggccgctaagagcacagcaatggaggaaacagctatatgggaacagcacacagtgacgcttcacagggctcctgggtttggatttggaattgcaatctctggtggaagagataatcctcattttcagagtggggaaacctccatagtgatttctgatgtgttaaaaggagggccagctgaaggacagctacaggaaaatgaccgagttgcaatggttaacggagtttcaatggataacgttgaacatgcttttgctgttcagcagctaaggaagagtgggaaaaacgcaaaaattactatccgaaggaagaagaaagttcagatccctgtaagtcacccagatcctgagccggtgtctgataatgaagacgatagttatgacgaagaagtgcatgacccaagagctggccgcggtgctttagcgaacagaaggagcgagaagagctgggcaagggataggagtgcaagcagggagaggagcctgtcccctcgctcggacaggcggtccgtggcctccagtcagcccgcaaagcccaccaaggtcacactggtgaagtctcggaaaaatgaagaatatggtcttcgattggccagccatatatttgtaaaggaaatttcacaagatagtttggcagcaagagatggtaacattcaagaaggggatgttgtcttgaagataaatggtactgtgacagaaaatatgtcattgacagatgcaaaaacactgatagaaaggtctaaaggcaagttaaaaatggtagtgcaaagagatgagcgggctaccttactgaacgtccctgacctttcggatagtatccattctgctaatgcctcggaaagagatgacatttcagaaattcagtcactagcatcagaccattcaggtcgctcgcatgacaggccaccccgccgcagccagtcacgatctcctgaccaacgttcagagccctccgatcattccacgcagtctccacagcagcccagcaatggcagtctccggagcagagaggaagagcgaatgtctaaacctggggccatctcaactcctgtaaaacatgtagacgatcatccacccaaagcagtggaagaagttacagttgagaaaaatgagaagcagacgcccactcttccagaaccgaaacctgtgtatgctcaagttggacaaccagatgtggatttacccgtcagcccttctgatggtgctctgcctaattcagctcatgaagacgggatacttaggcccagcatgaaactggtaaaattcagaaaaggagatagtgtgggtttgcgactagctggtggaaatgatgtcggaatatttgtagctggcgttctagaagatagccctgcagccaaagaaggcttagaggaaggtgatcaaattctcagggtgaacaatgtagatttcacaaatatcataagggaagaggccgtccttttcctccttgacctccctaaaggtgaagaagtgaccatactggctcagaagaagaaggacgtttatcgccgcattgtagaatcagatgtaggagattcattctatattagaacgcattttgaatatgaaaaagaatctccttacggacttagttttaacaaaggagaggtgttccgggtcgtggatactttatacaatggaaagctgggctcttggcttgccattcgaattggcaaaaatcataaggaggtagaacgaggcatcatccctaataagaacagagctgaacagttagccagtgtacagtacacacttccaaagacagcgggtggtgatcgggcagacttctggaggtttcgaggtcttcgcagctccaagagaaatcttcgaaaaagcagagaggacttgtcagctcagccagttcaaactaagttcccagcttatgaaagggttgttcttcgagaagctggattcctaagacctgtaaccatctttggaccaatagctgatgttgccagagaaaagttggcaagagaggagccagatatctatcagattgcaaaaagtgaaccacgagatgctgggactgaccatcgtagctctggcatcattcgccttcatacaataaagcaaatcatagatcaggataaacatgctttattagatgtaacgccaaatgcggttgatcgtcttaattatgcgcagtggtatccaattgttgtgttccttaaccctgactctaagcaaggtgtaaaaacaatgaggatgaggttgtgtccggagtctcggaaaagcgccaggaagctatatgaacgctctcataagcttcgtaagaacaatcaccatctcttcacaactacaattaacttaaactcaatgaatgatggttggtacggtgccctgaaagaagcgattcagcagcaacagaaccagctggtgtgggtctctgaggggaaggcggatggtgctacaagtgatgaccttgatttgcatgacgatcgtctgtcctacctgtcagccccaggtagtgagtactcaatgtatagcacggacagtagacacacttctgactatgaagacacagatacagaaggcggggcctacactgatcaagaactagatgaaactcttaatgatgaggtggggactcccccggagtctgccattacacggtcctctgagcctgtaagagaggattcctctggaatgcatcatgaaaaccagacataccctccttactcaccacaagcgcagccacaagctattcatagaatagactcccctggacttaagccagcctctcaacagaaagcagaagcctcatctccagtcccttacctttcgcctgaaacaaccccagcatcatcagcctctgcagttaatcataatgtcagtgtaactaatgtcagcctggaggagcctgccccagcccctcccacctcgcacgcatcacagcctggttgtttaggagcaccaagtgctgaggcagctcacgtagtgctcagaggtgaaggaccaccattgccgccgcatgcagacccagcaaaggtgtacaggaaggagccgtattctgaagaaatgatgagacaaaaccatattttaaaacaaccagctcttggtcacccagggcagaggccagataaagagccaaatctagcctatgaaccccaacttccatatatagaaaaacaagccagcagagaccttgagcagccgtcatacaggtatgaggtctcaagctacacagaccagttttctcggaactatgaccatcgcctacggtttgaagatcgaatccctacctatgaagaccagtggtcatattatgatgacaaacagccctaccaacctcggccttttgagaatcagcatccccgagacctggactccagacaacatcccgaagaggcttcagaacgaggttatttccagcgttttgaagagccagcccctctgtcgtacgacagtagaacacgctatgagcagctgcctcgaacctctactctacgacatgaagagcagccagcccctgcatatgaggtgcacaacaggtacaggccagaggcacagccctattcttcaacaggccctaagtcatctgagcccaagcagtactttgaccagtacccgcgaagttatgagcaagttccaccaccaggctttacctccaaaacaggccattacgagcctctccatggtgctgcagttgtccctcctctgataccttcctctcaacaaaagccagaagtcctgccctcggctaccaaaccacagcctccacccccaaccctaactgaggaggaggaggatccagcaatgaaaccacagtctgtgctcaccagagtcaaaatgtttgaaaacaaaagatctgcgtctttggagaacaagaaagatgtgaatgacactgccagctttaagcctccggaagtagcatctaaacctccaggtgcttctcttgctggccctaaacctgtccctcagagtcagtttagtgagcacgacaaaacgctctacaggctcccagagcctcagaaacctcaagtgaagccacccgaagatattgttcgatcaaatcattacgaccctgaagaggatgaagaatattaccggaaacagctctcctactttgaccgaagaagttttgagagcaagccttctgcacatcttcctgctggccatcactcagagcctgctaagccagtccattctcagagccagcccaacttctctagttactcttcaaagggaaaacccgaaactgatgctgtggatagatcattcagtgagaaacgttatgatccagcccaggccacgcctcctcctcctccgttgccctcacagtacagccagccagctccacctctgtccagctcttctctccacatacattccaagggcgcccagggtgaaggcaactcagtatcattggattttcagaactcatatatgtccaaaccagacccacccccatctcagagtaaaccagcaactttcagaccaccaactcgagaagacccccctcagactttctatccgcagaaaagtttcccagacaaagctccagttaacggagctgagcagactcagaaaaccatcactccggtgtacaatcgattcacaccaaagccgtacacaagttctgcccggccatttgaacgcaaatttgaaagtccgaagttcaaccataatcttctgccaagtgaaactgtacataaacctgaattgtcttcaaaaactcccacttccccaaaaactcttatgaaagctcatagttcaacacagcctccagagtttgacagtggagttgaaactttctctgttcacacagataagcctaaatatcaaatgaataatatcagcaccatgcctaaagctgtccctgtgagtccttcagctgtggaagaagatgaagatgaggatggtcatactgtagtggctacagctcgtggcatttttaacagcaatggtggtgtgttgagttccatagaaaccggtgttagtataattatcccacaaggagccattcctgaaggaattgagcaagaaatctatttcaaagtctgcagagacaatagcattctcccacctttagataaagagaaaggtgaaactctgctgagccccctagtgatgtgtgggccccatggcctcaagttcctgaagcccgtggagctacgcttgccacactgtgaccctaaaacctggcaaaacaagtgtcttcctggagatccgaattaccttgttggagccaactgtgtttctgtcctgattgaccacttttaattcttagtgtataggaactggactaagcaatgtgaacgtggattgaacttactaaatctaaatggaaccactctaatgagtattatattttcttagaatttatactgcaattggtagtattaagcatttgttggaactgatgaaggttagcgagcatgcccctgagccacggtcagaaaacatgctacaagctatgtgttattgagtgaagaactgtcaggcattggctagaggttcaaagatatttttgctttgtaatgatttttgtacttttttatggtcactgcttaacttcacatactgatttccgttaaaaataccagccagtaaatgggggtgcatttgagttctgttctttccaaagtacactcaaagtttattatggccttggcctagcatacacattttattttattatgcatgaggtaatgtgcacacattttttacaaatgcacctggaatatataaccagtatagtggatttaacagaaatgtacagcagggggatttataacttggggagggagggtcaaatgaagacaattacttattgtatatgaaaacacatttcttttagggaaggacaccaaagcatgtgagcccggttcatggcctctgaatctataaattaacatatcaccacagacatgtaaccagcaggaatgccttaccctagtgggtttaattcttccatcatttcgctgtgtattactaagtttttatgagttctgtgcatatctagatactgtaccatggaaaagactgagtagattgtggacttgatggtttcagaaatgtgcaaattacctggaagaaaataatggtgtgacttaaaatatttttttgacagggggagtggcagtcttctgttctttgggctgtgcatctgagatgtttgatgctttcaggacgcatgtacataatgcgtgcataccacttttgttcttggtttgtaaattaacttttataaactttacctttttatacataaacagaaccaagtttctaaggctacctttgtattctctcctgtacctcttgagccttgaactttgacctctgcagcaataaagcagcatttctacacatacaaggtcatttttctaagaaaaagaatgcacagagttgttacatttttaagtgctgcatttaaaagatacagttactctagtttgattaaattcttgcaaagtatcccttctgtaaaatttgtgatacaatactgtgccctaaagtgtatttttttactaatagacagtttattatggcacatcagcacgatttctgtttagataatacaccactacattctgttaataatcagtaggtgtgaaaggatttcttttgtggttattaaaaaaaaatctcaaatttctaaatctgcaagaataaaactttttaaaataca

SEQ ID NO: 115 - DNA, 7915 bp, Homo sapiens
  source 1..7915: mol_type = genomic DNA
gtggccgctgagttgccggcgccggctgagccagcggacgccgcgttccttggcggccgccggttcccgggaagttacgtggcgaagccggcttccgaggagacgccgggaggccacgggtgctgctgacgggcgggcgaccgggcgaggccgacgtggccgggctgcgaaagctgcgggaggccgagtgggtggccgcgctcggagggaggtgccggtcgggcgcgccccgtggagaagacccgggcggggcgggcgcttcccggacttttgtccgagttgaattccctccccctgggccgggcccttccggccgcccccgcccgtgccccgctcgctctcgggagatgtttatttgggctgtggcgtgaggagcgggcgggccagcgccgcggagtttcgggtccgaggagcctcgcgcggcgctggagagagacaagatgtccgccagagctgcggccgccaagagcacagcaatggaggaaacagctatatgggaacaacatacagtgacgcttcacagggctcctggatttggatttggaattgcaatatctggtggacgagataatcctcattttcagagtggggaaacgtcaatagtgatttcagatgtgctgaaaggaggaccagctgaaggacagctacaggaaaatgaccgagttgcaatggttaacggagtttcaatggataatgttgaacatgcttttgctgttcagcaactaaggaaaagtgggaaaaatgcaaaaattacaattagaaggaagaagaaagttcaaataccagtaagtcgtcctgatcctgaaccagtatctgataatgaagaagatagttatgatgaggaaatacatgatccaagaagtggccggagtggtgtggttaacagaaggagtgagaagatttggccgagggatagaagtgcaagtagagagaggagcttgtccccgcggtcagacaggcggtcagtggcttccagccagcctgctaaacctactaaagtcacactggtgaaatcccggaaaaatgaagaatatggtcttcgattggcaagccatatatttgttaaggaaatttcacaagatagtttggcagcaagagatggcaatattcaagaaggtgatgttgtattgaagataaatggtactgtgacagaaaatatgtcattgacagatgcaaagacattgatagaaaggtctaaaggcaaattaaaaatggtagttcaaagagatgaacgggctacgctattgaatgtccctgatctttctgacagcatccactctgctaatgcctctgagagagacgacatttcagaaattcagtcactggcatcagatcattctggtcgatcacacgataggcctccccgccgcagccggtcacgatctcctgaccagcggtcagagccttctgatcattccaggcactcgccgcagcagccaagcaatggcagtctccggagtagagatgaagagagaatttctaaacctggggctgtctcaactcctgtaaagcatgctgatgatcacacacctaaaacagtggaagaagttacagttgaaagaaatgagaaacaaacaccttctcttccagaaccaaagcctgtgtatgcccaagttgggcaaccagatgtggatttacctgtcagtccatctgatggtgtcctacctaattcaactcatgaagatgggattcttcggcccagcatgaaattggtaaaattcagaaaaggagatagtgtgggtttgcggctggctggtggaaatgatgttggaatatttgtagctggcgttctagaagatagccctgcagccaaggaaggcttagaggaaggtgatcaaattctcagggtaaacaacgtagattttacaaatatcataagagaagaagccgtccttttcctgcttgacctccctaaaggagaagaagtgaccatattggctcagaagaagaaggatgtttatcgtcgcattgtagaatcagatgtaggagattctttctatattagaacccattttgaatatgaaaaggaatctccctatggacttagttttaacaaaggagaggtgttccgtgttgtggataccttgtacaatggaaaactgggctcttggcttgctattcgaattggtaaaaatcataaggaggtagaacgaggcatcatccctaataagaacagagctgagcagctagccagtgtacagtatacacttccaaaaacagcaggcggagaccgtgctgacttctggagattcagaggtcttcgcagctccaagagaaatcttcgaaaaagcagagaggatttgtccgctcagcctgttcaaacaaagtttccagcttatgaaagagtggttcttcgagaagctggatttctgaggcctgtaaccatttttggaccaatagctgatgttgccagagaaaagctggcaagagaagaaccagatatttatcaaattgcaaagagtgaaccacgagacgctggaactgaccaacgtagctctggcattattcgcctgcatacaataaagcaaatcatagatcaagacaaacatgctttattagatgtaacaccaaatgcagttgatcgtcttaactatgcccagtggtatccaattgttgtatttcttaaccctgattctaagcaaggagtaaaaacaatgagaatgaggttatgtccagaatctcggaaaagtgccaggaagttatacgagcgatctcataaacttcgtaaaaataatcaccatctttttacaactacaattaacttaaattcaatgaatgatggttggtatggtgcgctgaaagaagcaattcaacaacagcaaaaccagctggtatgggtttccgagggaaaggcggatggtgctacaagtgatgaccttgatttgcatgatgatcgtctgtcctacctgtcagctccaggtagtgaatactcaatgtatagcacggacagtagacacacttctgactatgaagacacagacacagaaggcggggcctacactgatcaagaactagatgaaactcttaatgatgaggttgggactccaccggagtctgccattacacggtcctctgagcctgtaagagaggactcctctggaatgcatcatgaaaaccaaacatatcctccttactcaccacaagcgcagccacaaccaattcatagaatagactcccctggatttaagccagcctctcaacagaaagcagaagcttcatctccagtcccttacctttcgcctgaaacaaacccagcatcatcaacctctgctgttaatcataatgtaaatttaactaatgtcagactggaggagcccaccccagctccttccacctcttactcaccacaagctgattctttaagaacaccaagtactgaggcagctcacataatgctaagagatcaagaaccatcattgtcgtcgcatgtagatccaacaaaggtgtatagaaaggatccatatcccgaggaaatgatgaggcagaaccatgttttgaaacagccagccgttagtcacccagggcacaggccagacaaagagcctaatctgacctatgaaccccaactcccatacgtagagaaacaagccagcagagacctcgagcagcccacatacagatacgagtcctcaagctatacggaccagttttctcgaaactatgaacatcgtctgcgatacgaagatcgcgtccccatgtatgaagaacagtggtcatattatgatgacaaacagccctacccatctcggccaccttttgataatcagcactctcaagaccttgactccagacagcatcccgaagagtcctcagaacgagggtactttccacgttttgaagagccagcccctctgtcttacgacagcagaccacgttacgaacaggcacctagagcatccgccctgcggcacgaagagcagccagctcctgggtatgacacacatggtagactcagaccggaagcccagccccacccttcagcagggcccaagcctgcagagtccaagcagtattttgagcaatattcacgcagttacgagcaagtaccaccccaaggatttacctctagagcaggtcattttgagcctctccatggtgctgcagctgtccctccgctgataccttcatctcagcataagccagaagctctgccttcaaacaccaaaccactgcctccacccccaactcaaaccgaagaagaggaagatccagcaatgaagccacagtctgtactcaccagagttaagatgtttgaaaacaaaagatctgcatccttagagaccaagaaggatgtaaatgacactggcagttttaagcctccagaagtagcatctaaaccttcaggtgctcccatcattggtcccaaacccacttctcagaatcaattcagtgaacatgacaaaactctgtacaggatcccagaacctcaaaaacctcaactgaagccacctgaagatattgttcggtccaatcattatgaccctgaagaagatgaagaatattatcgaaaacagctgtcatactttgaccgaagaagttttgagaataagcctcctgcacacattgccgccagccatctctccgagcctgcaaagccagcgcattctcagaatcaatcaaatttttctagttattcttcaaagggaaagcctcctgaagctgatggtgtggatagatcatttggcgagaaacgctatgaacccatccaggccactccccctcctcctccattgccctcgcagtatgcccagccatctcagcctgtcaccagcgcgtctctccacatacattctaagggagcacatggtgaaggtaattcagtgtcattggattttcagaattccttagtgtccaaaccagacccacctccatctcagaataagccagcaactttcagaccaccaaaccgagaagatactgctcaggcagctttctatccccagaaaagttttccagataaagccccagttaatggaactgaacagactcagaaaacagtcactccagcatacaatcgattcacaccaaaaccatatacaagttctgcccgaccatttgaacgcaagtttgaaagtcctaaattcaatcacaatcttctgccaagtgaaactgcacataaacctgacttgtcttcaaaaactcccacttctccaaaaactcttgtgaaatcgcacagtttggcacagcctcctgagtttgacagtggagttgaaactttctctatccatgcagagaagcctaaatatcaaataaataatatcagcacagtgcctaaagctattcctgtgagtccttcagctgtggaagaggatgaagatgaagatggtcatactgtggtggccacagcccgaggcatatttaacagcaatgggggcgtgctgagttccatagaaactggtgttagtataattatccctcaaggagccattcccgaaggagttgagcaggaaatctatttcaaggtctgccgggacaacagcatccttccacctttagataaagagaaaggtgaaacactgctgagtcctttggtgatgtgtggtccccatggcctcaagttcctgaagcctgtggagctgcgcttaccacactgtgatcctaaaacctggcaaaacaagtgtcttcccggagatccaaattatctcgttggagcaaactgtgtttctgtccttattgaccacttttaactcttgaaatataggaacttaaataatgtgaaactggattaaacttaatctaaatggaaccactctatcaagtattataccttttttagagttgatactacagtttgttagtatgaggcatttgtttgaactgataaagatgagtgagcatgcccctgaaccatggtcggaaaacatgctacacactgcatgtttgtgattgacgggactgttggtattggctagaggttcaaagatattttgctttgtgatttttgtaatttttttatcgtcactgcttaacttcacatattgatttccgttaaaataccagccagtaaatgggggtgcatttgaggtctgttctttccaaagtacactgtttcaaactttactatggccctggcctagcatacgtacacattttattttattatgcatgaagtaatatgcacacattttttaaatgcacctggaatatataaccagtgttgtggatttaacagaaatgtacagcaaggagatttacaactgggggagggtgaagtgaagacaatgacttactgtacatgaaaacacatttttcttagggaaggatacaaaagcatgtgagactggttccatggcctcttcagatctctaacttcaccatattaccacagacatactaaccagcagaaatgccttaccctcatgttcttaattcttagctcattctccttgtgttactaagtttttatggcttttgtgcattatctagatactgtatcatgacaaagactgagtacgttgtgcatttggtggtttcagaaatgtgttatcacccagaagaaaatagtggtgtgatttggggatatttttttcttttcttttcttttctttttttttttttttgacaaggggcagtggtggttttctgttctttctggctatgcatttgaaaattttgatgttttaaggatgcttgtacataatgcgtgcataccacttttgttcttggtttgtaaattaacttttataaactttaccttttttatacataaacaagaccacgtttctaaaggctacctttgtattctctcctgtacctcttgagccttgaactttgacctctgcagcaataaagcagcgtttctatgacacatgcaaggtcattttttttaagaaaaaggatgcacagagttgttacatttttaagtgctgcatttaaaagatacagttactcagaattctctagtttgattaaattcttgcaaagtatccctactgtaatttgtgatacaatgctgtgccctaaagtgtatttttttactaatagacaatttattatggcacatcagcacgatttctgtttagataatacaccactacattctgttaatcattaggtgtgactgaatttcttttgccgttattaaaaatctcaaatttctaaatctccaaaataaaactttttaaaataaagtgctggcttggtctgtttgcccactgttttctagtttcatgcagctttataatcctgttttaaaatcctgcacacaaatccctatcacccagcgtcacctaccacctcgtcgtctggtgttgcatgcagaatttctccccttggccagcatgtacagatgggtgggcagtgctcatctgaagggctcagactgaagtggggcagaaggacctggagacagagtgggagaaggcagcaggccgacttccccctgtgggtaaacacacacccctgcgtggagaaacacccctgcatggggacacacgtgcgtttgtgtgtgtgcgtgtaaccatttgtatatggttttattccccagataaatagcacgggcattgtttaatgtcacccacatttgggggaagaaaatgggtttagtgagcataaatattccattgtggacatcctacttacttaactgccacccctgttgacatttggattattgctatagtgtttcagtaaacacctttgtggactgaatccatctatgtggattttatttttttcatctttgttaaggtataatttacatagagtaagttactgtttttaaaatgtatagttcaaattttgacaaacacatactgtttatttaccacggttaatacatacaacagtttctttatcttctctaatcatttgtacccctttatactcaacccttcttccagcccctggcaggatgatctcttctgtttctatagttgcctttttgacagtgtcatataaatggaatcatacaata